(19) United States
(12) Patent Application Publication
(10) Pub. No.: US 2024/0043490 A1
(43) Pub. Date: Feb. 8, 2024

(54) TARGETED CYTOKINE CONSTRUCT FOR ENGINEERED CELL THERAPY

(71) Applicant: ASHER BIOTHERAPEUTICS, INC., South San Francisco, CA (US)

(72) Inventors: Ivana DJURETIC, Pacifica, CA (US); Kelly MOYNIHAN, San Francisco, CA (US); Wei CHEN, Cupertino, CA (US); Paul BESSETTE, San Francisco, CA (US); Christopher KIMBERLIN, San Francisco, CA (US); Nathan MATHEWSON, San Francisco, CA (US); Yik Andy YEUNG, South San Francisco, CA (US)

(21) Appl. No.: 18/256,512

(22) PCT Filed: Dec. 8, 2021
(86) PCT No.: PCT/US2021/062484

§ 371 Date: Jun. 8, 2023

Related U.S. Application Data
(60) Provisional application No. 63/123,281, filed on Dec. 9, 2020.

(51) Int. Cl. C07K 14/55; A61K 39/00; C07K 14/54; A61P 35/00; C12N 5/078

(52) U.S. Cl. CPC C07K 14/55; A61K 39/4631; C07K 14/5418; C07K 14/5428; A61P 35/00; C12N 5/0634; A61K 2239/13; C12N 2510/00

(57) ABSTRACT

Provided herein are targeted cytokine constructs and method of engineered cell therapy by administering a targeted cytokine construct in combination with an engineered cell therapy, for use in treating a disease, e.g., a proliferative disease, e.g., a cancer.

### CROSS-REFERENCE

This application claims the benefit of U.S. Provisional Application No. 63/123,281, filed Dec. 9, 2020, which is incorporated herein by reference in its entirety.

### SUMMARY

One embodiment provides a targeted cytokine construct with an engineered cell comprising: a cell binding domain that targets at least one of: (i) a domain of a chimeric antigen receptor (CAR) or a T cell receptor (TCR) exogenously introduced into the engineered cell; (ii) a tag molecule selectively expressed on the surface of the engineered cell; (iii) a polypeptide tag that is part of a CAR exogenously introduced into the engineered cell; (iv) a polypeptide tag that is part of a TCR exogenously introduced into the engineered cell, or (vi) any combination of (i)-(v), and a cytokine protein or a functional fragment or a variant thereof.

In some embodiments the targeted cytokine construct selectively activates the engineered cell with 10-fold or greater potency as compared to activation of a non-engineered cell. In some embodiments the potency is measured by a pSTAT5 or a pSTAT3 activation assay. In some embodiments the domain of the CAR is an scFv. In some embodiments the non-engineered cell does not express on its surface: the CAR, the TCR, or the tag molecule. In some embodiments the cell binding domain comprises an antibody or an antigen binding fragment thereof. In some embodiments the antibody or an antigen binding fragment thereof is bivalent or monovalent. In some embodiments the cytokine is selected from the group consisting of: IL-2, IL-7, IL-10, IL-15, and IL-21, or a functional fragment thereof, or a variant thereof, or any combinations thereof. In some embodiments the cytokine is the IL-2 polypeptide, or a functional fragment thereof, or a variant thereof. In some embodiments the IL-2 polypeptide exhibits reduced binding affinity by at least about 50% to an IL-2Ra polypeptide having an amino acid sequence of SEQ ID NO:2, compared to the binding affinity of the wild-type IL-2 polypeptide with an amino acid sequence of SEQ ID NO:1. In some embodiments the IL-2 polypeptide exhibits reduced binding affinity by at least about 50% to an IL-2RP polypeptide having an amino acid sequence of SEQ ID NO:3, and/or reduced binding affinity by at least about 50% to an IL-2Ry polypeptide having an amino acid sequence of SEQ ID NO:4 compared to the binding affinity of the wild-type IL-2 polypeptide with an amino acid sequence of SEQ ID NO:1. In some embodiments the IL-2 polypeptide comprises the sequence of SEQ ID NO:1 with one or more amino acid substitutions relative to SEQ ID NO:1, and wherein the one or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130. In some embodiments the one or more substitution(s) comprise an F42A or F42K amino acid substitution relative to SEQ ID NO:1. In some embodiments the one or more substitution(s) further comprise an R38A, R38D, R38E, E62Q, E68A, E68Q, E68K, or E68R amino acid substitution relative to SEQ ID NO:1. In some embodiments the one or more substitution(s) further comprise an H16E, H16D, D20N, M23A, M23R, M23K, S87K, S87A, D84L, D84N, D84V, D84H, D84Y, D84R, D84K, N88A, N88G, N88S, N88T, N88R, N88I, N88D, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1. In some embodiments the one or more substitution(s) further comprise the amino acid mutation C125A compared to SEQ ID NO:1. In some embodiments the IL-2 polypeptide comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and N88G; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments the IL-2 polypeptide comprises an amino acid sequence that is at least about 85% identical to a sequence selected from the group consisting of SEQ ID Nos:11-90. In some embodiments the IL-2 polypeptide comprises a sequence that is at least about 75% identical to a sequence selected from the group consisting of SEQ ID Nos. 43, 48, 52, 49, and 156.

In some embodiments the cytokine is the IL-7 polypeptide, or a functional fragment thereof, or a variant thereof. In some embodiments the IL-7 polypeptide exhibits reduced binding affinity by at least about 50% to an IL-7Ra polypeptide comprising the amino acid sequence of SEQ ID NO: 94, compared to binding affinity of a wild-type IL-7 polypeptide comprising the amino acid sequence of SEQ ID NO: 91 to the IL-7Ra polypeptide. In some embodiments the IL-7 polypeptide exhibits reduced binding affinity by at least about 50% to an IL-2Rg polypeptide comprising the amino acid sequence of SEQ ID NO: 4, compared to binding affinity of a wild-type IL-7 polypeptide comprising the amino acid sequence of SEQ ID NO: 91 to the IL-2Rg polypeptide. In some embodiments the IL-7 polypeptide comprises the sequence of SEQ ID NO: 91, with one or more substitution relative to SEQ ID NO: 91. In some embodiments the one or more substitutions are at positions selected from the positions: K10, Q11, S14, V15, V18, Q22, L35, N36, D74, L77, L80, K81, E84, 188, R133, Q136, E137, T140, and N143, and K144. In some embodiments the substitution in position K81 is K81A and the substitution in position T140 is K140A. In some embodiments the cytokine is the IL-10 polypeptide, or a functional fragment thereof, or a variant thereof. In some embodiments the IL-10 polypeptide exhibits reduced binding affinity by at least about 50% to an IL-10RA polypeptide comprising the amino acid sequence of SEQ ID NO: 96, compared to binding affinity of a wild-type IL-10 polypeptide comprising the amino acid sequence of SEQ ID NO: 95 to the IL-10RA polypeptide. In some embodiments the IL-10 polypeptide exhibits increased binding affinity by at least about 50% to an IL-10RB polypeptide comprising the amino acid sequence of SEQ ID NO: 97, compared to binding affinity of a wild-type IL-10 polypeptide comprising the amino acid sequence of SEQ ID NO: 95 to the IL-10RB polypeptide. In some embodiments the IL-10 polypeptide comprises the sequence of SEQ ID NO: 95, with one or more substitution relative to SEQ ID NO: 95. In some embodiments the IL-10 polypeptide comprises an amino acid sequence selected from the group consisting of SEQ ID 99-112. In some embodiments the cytokine is the IL-21 polypeptide, or a functional fragment thereof, or a variant thereof. In some embodiments the IL-21 polypeptide exhibits reduced binding affinity by at least about 50% to an IL-21R polypeptide comprising the amino acid sequence of SEQ ID NO: 93, compared to binding affinity of a wild-type IL-21 polypeptide comprising the amino acid sequence of SEQ ID NO: 92 to the IL-21R polypeptide. In some embodiments the IL-21 polypeptide exhibits reduced binding affinity by at least about 50% to an IL-2Rg polypeptide comprising the amino acid sequence of SEQ ID NO: 4, compared to binding affinity of a wild-type IL-21 polypeptide comprising the amino acid sequence of SEQ ID NO: 92 to the IL-2Rg polypeptide. In some embodiments, the IL-21 polypeptide comprises the sequence of SEQ ID NO: 115, with one or more substitution relative to SEQ ID NO: 115. In some embodiments the substitution in one or more positions are selected from the positions: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115.

In some embodiments the engineered cell comprises at least one of: a T cell expressing a T cell receptor (a TCR-T cell), a gamma delta T cell, a pluripotent stem cell derived T cell, or an induced pluripotent stem cell derived T cell, a natural killer cell (NK cell), a pluripotent stem cell derived NK cell, or an induced pluripotent stem cell (iPSC) derived NK cell, a T cell engineered to express a chimeric antigen receptor (a CAR-T cell), a CD8-positive T cell, a CD4-positive T cell, a cytotoxic T cell, a tumor infiltrating lymphocyte, a CAR-NK cell, a gamma delta T cell, a myeloid cell, a hematopoietic lineage cell, a hematopoietic stem and progenitor cell (HSC), a hematopoietic multipotent progenitor cell (MPP), a pre-T cell progenitor cell, a T cell progenitor cell, a NK cell progenitor cell. In some embodiments the targeted cytokine construct is suitable for administering to a subject in combination with a therapy comprising the engineered cell. In some embodiments the engineered cell is autologous to the subject. In some embodiments the engineered cell is allogenic to the subject. In some embodiments the subject is human. In some embodiments the subject has a cancer. In some embodiments, the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

One embodiment provides a pharmaceutical composition: comprising a targeted cytokine construct according to this disclosure, and at least one of: a pharmaceutically acceptable excipient, carrier, or diluent, or any combination thereof. In some embodiments the pharmaceutical composition further comprises a population of engineered cells. One embodiment provides a cell therapy kit comprising a pharmaceutical composition that comprises a targeted cytokine construct according to this disclosure, and instructions specified for administering the targeted cytokine construct to a subject. In some embodiments, the cell therapy kit further comprises a pharmaceutical composition that comprises the population of engineered cells and instructions specified for administering the population of engineered cells to the subject. In some embodiments the pharmaceutical composition that comprises the targeted cytokine construct and the pharmaceutical composition that comprises the population of engineered cells are for sequential or simultaneous administration.

One embodiment provides a method of treating a condition in a subject, the method comprising administering to the subject a therapeutic regimen comprising: (a) an engineered cell and (b) a targeted cytokine construct that comprises: (i) a cell binding domain that binds a receptor or domain exogenously introduced into the engineered cell; and (ii) a cytokine protein or a functional fragment or a variant thereof.

In some embodiments the targeted cytokine construct selectively activates a population of engineered cells with 10-fold or greater potency as compared to activation of a population of non-engineered cells. In some embodiments administering the targeted cytokine construct results in an increase in activation of a population of engineered cells, as compared to the activation of a population of non-engineered cells. In some embodiments the activation is measured by a pSTAT5 or a pSTAT3 activation assay. In some embodiments the administering the targeted cytokine construct results in an increase in expansion and/or proliferation of a population of engineered cells, as compared to the expansion and/or proliferation of a population of non-engineered cells. In some embodiments the administering the targeted cytokine construct results in an increased in vivo persistence of a population of engineered cells, as compared to the in vivo persistence of a population of non-engineered cells. In some embodiments the non-engineered cells do not express: the CAR, the TCR, or the tag molecule. In some embodiments administering the targeted cytokine construct results in an increase in activation, expansion and/or proliferation of a population of engineered cells, as compared to the activation, expansion and/or proliferation of the population of engineered cells, when administered without the targeted cytokine construct. In some embodiments the expansion and/or proliferation is in vivo or in vitro. In some embodiments the administering the targeted cytokine construct results in an increased in vivo persistence of a population of the engineered cells, as compared to the in vivo persistence of the population of engineered cells, when administered without the targeted cytokine construct. In some embodiments the in vivo persistence of the population of engineered cells comprises a period of about 15 days, about 30 days to about a year. In some embodiments administering the targeted cytokine construct reduces a rate and/or extent of exhaustion of a population of the engineered cells, as compared to the rate and/or extent of exhaustion of the population of engineered cells, when administered without the targeted cytokine construct. In some embodiments administering the targeted cytokine construct results in selective potentiation of the engineered cells, allowing enhanced specific enrichment of a population of the engineered cells, as compared to specific enrichment of a population of engineered cells when administered with an untargeted cytokine or a functional fragment or a variant thereof. In some embodiments administering the targeted cytokine construct does not increase count of Treg cells in a biological sample isolated from the subject, as compared to the count of Treg cells in a biological sample isolated from a subject who has been administered an untargeted cytokine or a functional fragment or a variant thereof. In some embodiments the biological sample is at least one of: a tumor biopsy or peripheral blood. In some embodiments the subject is previously administered a pre-conditioning regimen. In some embodiments administering the targeted cytokine construct allows for reduction in at least one of: severity or duration of the pre-conditioning regimen. In some embodiments the pre-conditioning regimen is used to decrease the endogenous lymphocyte population so as to allow a population of the engineered cell to expand. In some embodiments the pre-conditioning regimen comprises administering a lymphodepletion agent. In some embodiments administering the targeted cytokine construct reduces the extent of lymphodepletion required for engraftment of the engineered cell. In some embodiments the pre-conditioning regimen involves administering a chemotherapeutic agent to the subject. In some embodiments the chemotherapeutic agent is at least one of: fludarabine and cyclophosphamide. In some embodiments the pre-conditioning regimen comprises a radiation treatment. In some embodiments the pre-conditioning regimen comprises administering a depleting antibody. In some embodiments the depleting antibody is alemtuzumab. In some embodiments the subject is not administered a pre-conditioning regimen.

One embodiment provides a method of eliminating the need for administering, or minimizing the severity of, a pre-conditioning regimen prior to administering an engineered cell therapy, the method comprising, administering to a subject a therapeutic regimen comprising: (a) an engineered cell; and (b) a targeted cytokine construct that comprises: (i) a cell binding domain that binds a receptor or domain exogenously introduced into the engineered cell; and (ii) a cytokine protein or a functional fragment or a variant thereof.

In some embodiments the subject has not been administered a pre-conditioning regimen. In some embodiments the targeted cytokine construct selectively activates engineered cells with 10-fold or greater potency as compared to activation of non-engineered cells. In some embodiments the activation is measured by a pSTAT5 or pSTAT3 activation assay. In some embodiments the non-engineered cells do not comprise a receptor or domain exogenously introduced into the cells. In some embodiments the targeted cytokine construct is administered within or within about 2 days, 3 days, 6 days, 12 days, 15 days, 30 days, 60 days or 90 days or more following administering the engineered cell. In some embodiments the targeted cytokine construct is administered simultaneously with administering the engineered cell. In some embodiments an effective dose of the engineered cell in the therapeutic regimen is lower than that of a reference therapeutic regimen that comprises administering the engineered cell but does not comprise administering the targeted cytokine construct.

In some embodiments the effective dose of the engineered cell in the therapeutic regimen is at least about 1.5× to about 1000× lower than the effective dosage of the engineered cell in the reference therapeutic regimen.

One embodiment provides a method of increasing the efficacy of an engineered cell therapy in a subject, the method comprising: administering to a subject a therapeutic regimen comprising: (a) the engineered cell; and (b) a targeted cytokine construct comprising: (i) a cell binding domain that binds a receptor or domain exogenously introduced into the engineered cell; and (ii) a cytokine protein or a functional fragment or a variant thereof, thereby increasing the efficacy of the engineered cell therapy in the subject.

In some embodiments the targeted cytokine construct selectively activates engineered cells with 10-fold or greater potency as compared to activation of non-engineered cells. In some embodiments the non-engineered cells do not comprise a receptor or domain exogenously introduced into the cells. In some embodiments the wherein the potency is measured by a pSTAT5 activation assay.

One embodiment provides a method of treating a subject who has undergone a loss of B cell aplasia, the method comprising: administering to the subject a targeted cytokine construct that comprises: (i) a cell binding domain that binds a receptor or domain exogenously introduced into the engineered cell; and (ii) a cytokine protein or a functional fragment or a variant thereof.

One embodiment provides a method of treating a condition or disease, comprising administering to the subject a therapeutic regimen comprising: (a) an engineered cell; and (b) a targeted cytokine construct that comprises: (i) a cell binding domain that binds a receptor or domain exogenously introduced into the engineered cell; and (ii) a cytokine protein or a functional fragment or a variant thereof, wherein the administering the targeted cytokine construct allows for reducing an effective dose of the engineered cell in the therapeutic regimen, relative to the effective dose of the engineered cell in a reference therapeutic regimen that comprises administering the engineered cell but does not comprise the targeted cytokine construct.

In some embodiments the effective dose of the engineered cell in the therapeutic regimen is at least about 1.5× to about 1000× lower than the effective dosage of the engineered cell in the reference therapeutic regimen. In some embodiments the engineered cell is provided in a composition, and wherein the composition is generated at a point-of-care and is administered into a patient without culturing the population of cells. One embodiment provides a method of targeting an engineered cell in a subject, the method comprising, administering to the subject a targeted cytokine construct comprising a cell binding domain and a modified cytokine or a functional fragment or a variant thereof, wherein the engineered cell expresses (i) a receptor for the modified cytokine or a functional fragment or a variant thereof, and (ii) a target antigen for the cell binding domain. One embodiment provides a method of enriching a population of an engineered cell in a subject, the method comprising: administering to the subject a targeted cytokine construct comprising a cell binding domain and a modified cytokine or a functional fragment or a variant thereof, wherein the engineered cell expresses (i) a receptor for the modified cytokine or a functional fragment or a variant thereof, and (ii) a target antigen for the cell binding domain.

In some embodiments the engineered cell is generated in vivo in the subject. In some embodiments the subject has previously been administered a nucleic acid carrier, comprising a nucleic acid that expresses a chimeric antigen receptor (CAR) or a T cell receptor protein (TCR). In some embodiments the nucleic acid carrier is at least one of: a linear polynucleotide, a polynucleotide associated with ionic or amphiphilic compounds, a plasmid, and a virus. In some embodiments the nucleic acid carrier is a nanocarrier. In some embodiments the nucleic acid carrier is a viral vector, wherein the viral vector is at least one of: a Sendai viral vector, an adenoviral vector, an adeno-associated virus vectors, a retroviral vector, or a lentiviral vector. In some embodiments the nucleic acid is a DNA or an RNA. In some embodiments the RNA is a messenger RNA (mRNA). In some embodiments the nucleic acid carrier further comprises a targeting moiety for targeting an immune cell. In some embodiments, the immune cell comprises, a myeloid cell, a T cell or an NK cell. In some embodiments, the T cell comprises a T lymphocyte. In some embodiments, the T cell or the NK cell is induced by the vector or the nucleic acid carrier, to generate the engineered cell in vivo in the subject. In some embodiments, administering the targeted cytokine construct results in an increase in activation, expansion and/or proliferation of a population of engineered cells generated in vivo, as compared to the activation, expansion and/or proliferation of the population of the engineered cells generated in vivo, when the subject is not administered the targeted cytokine construct.

In some embodiments, administering the targeted cytokine construct results in an increase in persistence of a population of engineered cells generated in vivo, as compared to the persistence of the population of the engineered cells generated in vivo, when the subject is not administered the targeted cytokine construct. In some embodiments, administering the targeted cytokine construct reduces a rate and/or extent of exhaustion of a population of engineered cells generated in vivo, as compared to the rate and/or extent of exhaustion of the population of the engineered cell generated in vivo, when administered without the targeted cytokine construct. In some embodiments, administering the targeted cytokine construct results in selective potentiation of the engineered cells generated in vivo, allowing enhanced specific enrichment of a population of the engineered cells generated in vivo, as compared to specific enrichment of a population of engineered cells when administered with an untargeted cytokine or a functional fragment or a variant thereof. In some embodiments, administering the targeted cytokine construct does not increase count of Treg cells in a biological sample isolated from the subject, as compared to the count of Treg cells in a biological sample isolated from a subject who has been administered an untargeted cytokine or a functional fragment or a variant thereof. In some embodiments, the biological sample is at least one of: a tumor biopsy or peripheral blood. In some embodiments, the persistence of the population of engineered cells comprises a period of at least about 30 days to about a year.

One embodiment provides a method of enriching a population of an engineered cell, the method comprising: contacting the population of the engineered cell with a targeted cytokine construct comprising a cell binding domain and a modified cytokine or a functional fragment or a variant thereof, wherein the engineered cell expresses (i) a receptor for the modified cytokine or a functional fragment or a variant thereof, and (ii) a target antigen for the cell binding domain. In some embodiments, the cytokine is selected from the group consisting of: IL-2, IL-7, IL-10, IL-15, and IL-21, or a functional fragment thereof, or a variant thereof, or any combinations thereof. In some embodiments, the cytokine is at least one of: (i) an IL-2Rβγ agonist polypeptide that binds to and/or activates an IL-2RP polypeptide comprising the amino acid sequence of SEQ ID NO: 3; and (ii) an IL-2Rβγ polypeptide agonist polypeptide that binds to and/or activates an IL-2Ry polypeptide comprising the amino acid sequence of SEQ ID NO: 4. In some embodiments, the cytokine is an IL-2 polypeptide, or a functional fragment thereof, or a variant thereof. In some embodiments, the method of claim 114, wherein said the IL-2 polypeptide exhibits reduced binding affinity by at least about 50% to an IL-2Ra polypeptide comprising the amino acid sequence of SEQ ID NO: 2, compared to binding affinity of a wild-type IL-2 polypeptide comprising the amino acid sequence of SEQ ID NO:1 to the IL-2Ra polypeptide. In some embodiments, the IL-2 polypeptide exhibits reduced binding affinity by at least about 50% to an IL-2RP polypeptide comprising the amino acid sequence of SEQ ID NO: 3, and/or reduced binding affinity by at least about 50% to an IL-2Ry polypeptide comprising the amino acid sequence of SEQ ID NO:4, compared to binding affinity of a wild-type IL-2 polypeptide comprising the amino acid sequence of SEQ ID NO: 1 to the IL-2RP polypeptide. In some embodiments, the cytokine is an IL-7 polypeptide that exhibits reduced binding affinity by at least about 50% to an IL-7Ra polypeptide comprising the amino acid sequence of SEQ ID NO: 94, compared to binding affinity of a wild-type IL-7 polypeptide comprising the amino acid sequence of SEQ ID NO: 91 to the IL-7Ra polypeptide. In some embodiments, the IL-7 polypeptide exhibits reduced binding affinity by 50% or more to an IL-2Rg polypeptide comprising the amino acid sequence of SEQ ID NO: 4, compared to binding affinity of a wild-type IL-7 polypeptide comprising the amino acid sequence of SEQ ID NO: 91 to the IL-2Rg polypeptide. In some embodiments, the cytokine is a IL-10 polypeptide that exhibits reduced binding affinity by at least about 50% to an IL-10RA polypeptide comprising the amino acid sequence of SEQ ID NO: 96, compared to binding affinity of a wild-type IL-10 polypeptide comprising the amino acid sequence of SEQ ID NO: 95 to the IL-10RA polypeptide. In some embodiments, the IL-10 polypeptide exhibits increased binding affinity by at least about 50% to an IL-10RB polypeptide comprising the amino acid sequence of SEQ ID NO: 97, compared to binding affinity of a wild-type IL-10 polypeptide comprising the amino acid sequence of SEQ ID NO: 95 to the IL-10RB polypeptide. In some embodiments, the cytokine is a IL-21 polypeptide that exhibits reduced binding affinity by 50% or more to an IL-21R polypeptide comprising the amino acid sequence of SEQ ID NO: 93, compared to binding affinity of a wild-type IL-21 polypeptide comprising the amino acid sequence of SEQ ID NO: 92 to the IL-21R polypeptide. In some embodiments, the IL-21 polypeptide exhibits reduced binding affinity by at least about 50% to an IL-2Rg polypeptide comprising the amino acid sequence of SEQ ID NO:4, compared to binding affinity of a wild-type IL-21 polypeptide comprising the amino acid sequence of SEQ ID NO: 92 to the IL-2Rg polypeptide. In some embodiments, the cytokine is an IL-2 polypeptide that comprises the sequence of SEQ ID NO:1 with one or more amino acid substitutions relative to SEQ ID NO:1, and wherein the one or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130. In some embodiments, the one or more substitution(s) comprise an F42A or F42K amino acid substitution relative to SEQ ID NO:1. In some embodiments, the one or more substitution(s) further comprise an R38A, R38D, R38E, E62Q, E68A, E68Q, E68K, or E68R amino acid substitution relative to SEQ ID NO:1. In some embodiments, the one or more substitution(s) further comprise an H16E, H16D, D20N, M23A, M23R, M23K, S87K, S87A, D84L, D84N, D84V, D84H, D84Y, D84R, D84K, N88A, N88G, N88S, N88T, N88R, N88I, N88D, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1. In some embodiments, the one or more substitution(s) further comprise the amino acid mutation C125A compared to SEQ ID NO:1. In some embodiments, the cytokine is an IL-2 polypeptide that comprises an amino acid sequence that is at least about 85% identical to a sequence selected from the group consisting of SEQ ID Nos:11-90. In some embodiments, the cytokine is an TL-2 polypeptide that comprises an amino acid sequence that is at least about 85% identical to a sequence selected from the group consisting of SEQ ID NOs: 43, 48, 52, 49, and 156. In some embodiments, the cytokine is an IL-2 polypeptide that comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and N88G; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

In some embodiments, the cytokine is an IL-7 polypeptide, or a functional fragment or a variant thereof. In some embodiments, the IL-7 polypeptide comprises the sequence of SEQ ID NO: 91, with one or more substitution relative to SEQ ID NO: 91. In some embodiments, the substitution in one or more positions are selected from the positions: K10, Q11, S14, V15, V18, Q22, L35, N36, D74, L77, L80, K81, E84, 188, R133, Q136, E137, T140, and N143, and K144. In some embodiments, the substitution in position K81 is K81A and the substitution in position T140 is K140A. In some embodiments, the cytokine is an IL-10 polypeptide, or a functional fragment or a variant thereof. In some embodiments, the IL-10 polypeptide comprises the sequence of SEQ ID NO: 95, with one or more substitution relative to SEQ ID NO: 95. In some embodiments, the mutant IL-10 polypeptide comprises an amino acid sequence selected from the group consisting of SEQ ID 99-112. In some embodiments, the cytokine is an IL-21 polypeptide, or a functional fragment thereof, or a variant thereof. In some embodiments, the IL-21 polypeptide comprises the sequence of SEQ ID NO: 115, with one or more substitution relative to SEQ ID NO: 115. In some embodiments, the substitution in one or more positions are selected from the positions: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115.

In some embodiments, the engineered cell comprises at least one of: a T cell expressing a T cell receptor (a TCR-T cell), a gamma delta T cell, a pluripotent stem cell derived T cell, or an induced pluripotent stem cell derived T cell, a natural killer cell (NK cell), a pluripotent stem cell derived NK cell, or an induced pluripotent stem cell (iPSC) derived NK cell, a T cell engineered to express a chimeric antigen receptor (a CAR-T cell), a CD8-positive T cell, a CD4-positive T cell, a cytotoxic T cell, a tumor infiltrating lymphocyte, a CAR-NK cell, a gamma delta T cell, a myeloid cell, a hematopoietic lineage cell, a hematopoietic stem and progenitor cell (HSC), a hematopoietic multipotent progenitor cell (MPP), a pre-T cell progenitor cell, a T cell progenitor cell, a NK cell progenitor cell.

In some embodiments, the engineered cell is autologous to the subject. In some embodiments, the engineered cell is allogenic to the subject. In some embodiments, the subject is human. In some embodiments, the subject has a cancer. In some embodiments, the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

One embodiment provides a targeted cytokine construct for use in a combination therapy with an engineered cell, the fusion protein comprising (i) a cell binding domain, and (ii) a cytokine protein or a functional fragment or a variant thereof, wherein the cell binding domain:
(a) comprises an antibody or an antigen binding fragment thereof that is specific for a receptor or domain exogenously expressed on the surface of the engineered cell;
(b) comprises an antibody or an antigen binding fragment thereof that is specific for a domain of an antigen binding protein expressed on the engineered cell;
(c) is specific for a tag, wherein the tag is co-expressed by the engineered cell or is part of a receptor expressed by the engineered cell;
(d) is a domain from an antigen targeted by the engineered cell; or
(e) comprises any combinations of (a)-(d).

In some embodiments, the receptor expressed by the engineered cell is a chimeric antigen receptor (CAR) or a T cell receptor (TCR). In some embodiments, the cytokine is selected from the group consisting of: IL-2, IL-7, IL-10, IL-15, and IL-21, or a functional fragment thereof, or a variant thereof, or any combinations thereof. In some embodiments, the cytokine is an IL-2 polypeptide, or a functional fragment thereof, or a variant thereof. In some embodiments, the IL-2 polypeptide comprises the sequence of SEQ ID NO:1 with one or more amino acid substitutions relative to SEQ ID NO:1, and wherein the one or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130. In some embodiments, the one or more substitution(s) comprise an F42A or F42K amino acid substitution relative to SEQ ID NO:1. In some embodiments, the one or more substitution(s) further comprise an R38A, R38D, R38E, E62Q, E68A, E68Q, E68K, or E68R amino acid substitution relative to SEQ ID NO:1. In some embodiments, the one or more substitution(s) further comprise an H16E, H16D, D20N, M23A, M23R, M23K, S87K, S87A, D84L, D84N, D84V, D84H, D84Y, D84R, D84K, N88A, N88G, N88S, N88T, N88R, N88I, N88D, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1. In some embodiments, the one or more substitution(s) further comprise the amino acid mutation C125A compared to SEQ ID NO:1. In some embodiments, the IL-2 polypeptide comprises an amino acid sequence that is at least about 85% identical to a sequence selected from the group consisting of SEQ ID Nos: 11-90. In some embodiments, the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and N88G; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the cytokine is an IL-7 polypeptide that comprises the sequence of SEQ ID NO: 91, with one or more substitution relative to SEQ ID NO: 91. In some embodiments, the substitution in one or more positions are selected from the positions: K10, Q11, S14, V15, V18, Q22, L35, N36, D74, L77, L80, K81, E84, 188, R133, Q136, E137, T140, and N143, and K144. In some embodiments, the substitution in positions K81 and T140 are K81A and T140A. In some embodiments, the cytokine is an IL-10 polypeptide comprises the sequence of SEQ ID NO: 95, with one or more substitution relative to SEQ ID NO: 95. In some embodiments, the IL-10 polypeptide comprises an amino acid sequence selected from the group consisting of SEQ ID 99-112. In some embodiments, the cytokine is an IL-21 polypeptide, or a functional fragment thereof, or a variant thereof. In some embodiments, the IL-21 polypeptide comprises the sequence of SEQ ID NO: 115, with one or more substitution relative to SEQ ID NO: 115. In some embodiments, the IL-21 polypeptide comprises the sequence of SEQ ID NO: 115, or a sequence comprising an amino acid substitution at one or more positions selected from the group consisting of positions: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115. In some embodiments, the tag co-expressed by the engineered cell is an EGFRt tag. In some embodiments, the antigen targeted by the engineered cell is selected from the group consisting of: a neoepitope from a tumor-associated antigen, TSHR, CD19, CD123, CD22, CD30, CD171, CS-1, CLL-1, CD33, EGFRvlll, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11 Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51 E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1 B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1.

In some embodiments, the engineered cell comprises at least one of: a T cell expressing a alpha beta T cell receptor, a gamma delta T cell, an NK T cell, a regulatory T cell, a pluripotent stem cell derived T cell, or an induced pluripotent stem cell derived T cell, a natural killer cell (NK cell), a pluripotent stem cell derived NK cell, or an induced pluripotent stem cell (iPSC) derived NK cell, a T cell engineered to express a chimeric antigen receptor (a CAR-T cell), a T cell engineered to express a T cell receptor (a TCR-T cell), a CD8-positive T cell, a CD4-positive T cell, a cytotoxic T cell, a tumor infiltrating lymphocyte, an NK cell engineered to express a chimeric antigen receptor (a CAR-NK cell), an NK T cell engineered to express a chimeric antigen receptor (a CAR-NK T cell), a myeloid cell, a hematopoietic lineage cell, a hematopoietic stem and progenitor cell (HSC), a hematopoietic multipotent progenitor cell (MPP), a pre-T cell progenitor cell, a T cell progenitor cell, a NK cell progenitor cell.

One embodiment provides a method of treating a cancer, the method comprising administering a targeted cytokine construct according to any one of claims 147-168, in a combination therapy with the engineered cell. In some embodiments, the further comprises administering an additional therapeutic agent. In some embodiments, the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

One embodiment provides a pharmaceutical composition: comprising a targeted cytokine construct according to this disclosure, and at least one of: a pharmaceutically acceptable excipient, carrier, or diluent, or any combination thereof. In some embodiments, the further comprises a population of the engineered cell. One embodiment provides a cell therapy kit that has a pharmaceutical composition that comprises a targeted cytokine construct according to this disclosure and instructions specified for administering the targeted cytokine construct to a subject. In some embodiments, the cell therapy kit further comprises a pharmaceutical composition that comprises a population of the engineered cells and instructions specified for administering the population of engineered cells to the subject. In some embodiments, the pharmaceutical composition that comprises the targeted cytokine construct and the pharmaceutical composition that comprises the population of engineered cells are for sequential or simultaneous administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the general mechanism for how targeted cytokine constructs containing a cytokine polypeptide (e.g., a wild-type cytokine protein or a fragment or a variant thereof, such as a mutein cytokine, which, in some examples, is a mutated IL-2 (also referred to as a mutated I1L2, or a mutein IL-2, or a mutein TL2)) with a cell binding domain that recognizes a target antigen (a domain or receptor) selectively expressed on an engineered cell (e.g., a CAR transduced T cell), or untargeted cytokine polypeptides work to stimulate engineered cells expressing target antigens or other cells not expressing target antigens.

FIG. 2 depicts the results of an assay demonstrating preferential activity of an exemplary targeted cytokine construct containing a cell binding domain that is an anti-CAR antibody, specifically, results for a phospho-STAT5 assay with engineered cells (CAR-T cells) generated from one donor cultured with the indicated constructs: a non-CAR binding antibody fused to an IL-2Rβγ-binding polypeptide IL2m1 (control IL2 m1), a CAR-binding antibody fused to an IL-2Rβγ-binding polypeptide IL2m1 (anti-CAR IL2m1), or an untargeted cytokine (IL-2) as a control. Percent pSTAT5-expressing cells was depicted for either CAR+ or CAR− T cells at the indicated concentrations.

FIG. 3 depicts the results of an assay demonstrating preferential activity of an exemplary targeted cytokine construct containing a cell binding domain that is an anti-CAR antibody, specifically, the results for a phospho-STAT5 assay with engineered cells (CAR-T cells) generated from a separate donor as FIG. 2 cultured with the indicated constructs: a non-CAR binding antibody fused to an IL-2Rβγ-binding polypeptide IL2m2 (control IL2m2), a CAR-binding antibody fused to an IL-2Rβγ-binding polypeptide IL2m2 (anti-CAR IL2m2), or an untargeted cytokine (IL-2) as a control. Percent pSTAT5-expressing cells was depicted for either CAR+ or CAR− T cells at the indicated concentrations.

FIG. 4 shows the frequency (left panel) and number (right panel) of CAR+ T cells in culture achieved with prolonged culture with either an untargeted cytokine (IL-2) or a targeted cytokine construct containing a cell binding domain that is an anti-CAR antibody and a an IL-2Rβγ-binding polypeptide (IL2m1 or IL2m2). Anti-CAR IL2 m1, Anti-CAR IL2m2, control IL-2, or an Anti-CAR antibody which is a cell binding domain without a cytokine, were cultured for up to 10 days in culture at a concentration of 0.1 nM. CAR+ T cell numbers and frequencies were measured by flow cytometry at the indicated time points.

FIG. 5A shows exemplary designs for CAR-targeted cytokine constructs targeting CAR-transduced engineered T cells according to this disclosure.

FIG. 5B shows exemplary designs for TCR-targeted cytokine constructs targeting TCR-transduced engineered T cells according to this disclosure.

FIGS. 6A-6D show the amino acid sequences of mature IL-2 (FIG. 6A; SEQ ID NO:1), IL-2Ra (FIG. 6B; SEQ ID NO:2), IL-2RP (FIG. 6C; SEQ ID NO:3) and IL-2Ry (FIG. 6D; SEQ ID NO:4) polypeptides.

FIG. 7 shows the amino acid sequence of wild-type mature IL-2 polypeptide (SEQ ID NO:1). “X” denotes the amino acid substituted in the sequence of wild-type IL-2 polypeptide for another amino acid to generate mutant IL-2 polypeptides of the present disclosure.

FIGS. 8A-8C show schematics of three different exemplary CAR-targeted cytokine constructs according to this disclosure, respectively. FIG. 8A shows a bivalent antibody with a mutant IL-2 polypeptide fused to the C-terminal of one of the heavy chains. FIG. 8B shows a monovalent antibody with a mutant IL-2 polypeptide fused to the C-terminal of the heavy chain that lacks variable region. FIG. 8C shows a monovalent antibody with a mutant IL-2 polypeptide fused to the N-terminal of the heavy chain that lacks variable region.

FIGS. 9A-1D show the amino acid sequences of the following polypeptides: mature IL-10 (FIG. 9A; SEQ ID NO: 95), IL-10RA (FIG. 9B; SEQ ID NO: 96), IL-10RB (FIG. 9C; SEQ ID NO: 97), and mature monomer IL-10 (FIG. 9D; SEQ ID NO: 98).

FIGS. 10A-10B show the amino acid sequences of the wild-type mature IL-10 polypeptide (FIG. 10A; SEQ ID NO: 95) and the mature monomer IL-10 (FIG. 10B; SEQ ID NO: 98). “X” denotes the amino acid substituted in the sequence of wild-type IL-10 polypeptide for another amino acid to generate the mutant IL-10 polypeptides of the present disclosure.

FIGS. 11A-11B show the amino acid sequences of the wild-type mature IL-10 polypeptide (FIG. 11A; SEQ ID NO: 95) and the mature monomer IL-10 (FIG. 11B; SEQ ID NO: 98). White boxes denote the residues that were substituted to reduce IL-10 affinity to IL-10RA, grey shaded boxes denote the residues that were substituted to modify IL-10 affinity to IL-10RB. Amino acids that were substituted in place of wild-type residues for each position are shown.

FIGS. 12A-12F depict the results of an assay demonstrating preferential activity of several exemplary targeted cytokine constructs containing a cell binding domain that is an anti-scFv antibody (targeting an scFv expressed by the CAR T cell) and TL-2 muteins (m1 FIG. 12B; m2 FIG. 12C; m3 FIG. 12D; m4 FIG. 12E; m5 FIG. 12F), specifically, results for a phospho-STAT5 assay with engineered cells (CAR-T cells (CAR+) in a mixture with CAR-nonexpressing T cells (CAR-)) generated from one donor cultured with the indicated constructs. Percent pSTAT5-expressing cells was depicted for either CAR+ or CAR− T cells at the indicated concentrations. FIG. 12A shows an illustration of the construct tested in this assay.

FIGS. 13A-13F depict the results of an assay demonstrating preferential activity of several exemplary targeted cytokine constructs containing a cell binding domain that is an anti-tag antibody (targeting a tag molecule selectively expressed by the CAR-T cell) and IL-2 muteins (m1 FIG. 13B; m2 FIG. 13C; m3 FIG. 13D; m4 FIG. 13E; m5 FIG. 13F), specifically, results for a phospho-STAT5 assay with engineered cells (CAR-T cells (CAR+) in a mixture with CAR-nonexpressing T cells (CAR-)) generated from one donor cultured with the indicated constructs. Percent pSTAT5-expressing cells was depicted for either CAR+ or CAR− T cells at the indicated concentrations. FIG. 13A shows an illustration of the construct tested in this assay.

FIGS. 14A-14F depict the results of an assay demonstrating preferential activity of several exemplary targeted cytokine constructs containing a cell binding domain that is an anti-tag antibody (targeting a tag expressed by the CAR-T cell) and TL-2 muteins (m1 FIG. 14B; m2 FIG. 14C; m3 FIG. 14D; m4 FIG. 14E; m5 FIG. 14F), specifically, results for a phospho-STAT5 assay with engineered cells (CAR-T cells) generated from one donor cultured with the indicated constructs. Percent pSTAT5-expressing cells was depicted for either CAR+ or CAR− T cells at the indicated concentrations. FIG. 14A shows an illustration of the construct tested in this assay.

FIGS. 15A-15B depict the results of an assay demonstrating preferential binding of an exemplary targeted cytokine construct containing a cell binding domain that is an anti-tag antibody (targeting a tag expressed by TCR-transduced T cells). FIG. 15A shows an illustration of a targeted cytokine construct containing the anti-tag antibody used in FIG. 15B with an TL-2 mutein.

### DETAILED DESCRIPTION

### Certain Definitions

As used in the specification and claims, the singular forms “a,” “an,” and “the” include plural references unless the context clearly dictates otherwise. For example, the term “a chimeric transmembrane receptor polypeptide” includes a plurality of chimeric transmembrane receptor polypeptides.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

A “cytokine” is a form of immunomodulatory polypeptide that can mediate cross-talk between initiating/primary cells and target/effector cells. It can function as a soluble form or cell-surface associated to bind the “cytokine receptor” on target immune cells to activate signaling. “Cytokine receptor” as used here is the polypeptide on the cell surface that activates intracellular signaling upon binding the cytokine on the extracellular cell surface. Cytokines can include, but are not limited to, chemokines, interferons, interleukins, lymphokines, and tumor necrosis factors. Cytokines are produced by a wide range of cells, including immune cells, endothelial cells, fibroblasts, and stromal cells. A given cytokine may be produced by more than one cell type. Cytokine are pleiotropic; since the receptors are expressed on multiple immune cell subsets, one cytokine can activate the signaling pathway in multiple cells. However, depending on the cell type, the signaling events for a cytokine can result in different downstream cellular events such as activation, proliferation, survival, apoptosis, effector function and secretion of other immunomodulatory proteins. A given cytokine, in some embodiments, is a wild-type cytokine polypeptide, a fragment thereof, or a variant thereof, such as a mutated cytokine polypeptide (also referred to herein as a mutein cytokine, e.g., a mutein IL-2, a mutein IL-7).

An “antigen binding domain” as used here refers to a molecule that specifically binds an antigenic determinant. A targeting moiety or an antigen binding domain may be a protein, carbohydrate, lipid, or other chemical compound. It can include, but is not limited to, antibody, antibody fragments (Chames et al, 2009; Chan & Carter, 2010; Leavy, 2010; Holliger & Hudson, 2005), scaffold antigen binding proteins (Gebauer and Skerra, 2009; Stumpp et al, 2008), single domain antibodies (sdAb), minibodies (Tramontano et al, 1994), the variable domain of heavy chain antibodies (nanobody, VHH), the variable domain of the new antigen receptors (VNAR), carbohydrate binding domains (CBD) (Blake et al, 2006), collagen binding domain (Knight et al, 2000), lectin binding proteins (Tetranectin), collagen binding proteins, adnectin/fibronectin (Lipoviek, 2011), a serum transferrin (trans-body), Evibody, Protein A-derived molecule, such as Z-domain of Protein A (Affibody) (Nygren et al, 2008), an A-domain (Avimer/Maxibody), alphabodies (WO2010066740), Avimer/Maxibody, designed ankyrin-repeat domains (DARPins) (Stumpp et al, 2008), anticalins (Skerra et al, 2008), a human gamma-crystallin or ubiquitin (Affilin molecules), a kunitz type domain of human protease inhibitors, knottins (Kolmar et al, 2008), linear or constrained peptide with or without fusion to extend half-life e.g. (Fc fusion-Peptibody) (Rentero Rebollo & Heinis, 2013; EP 1144454 B2; Shimamoto et al, 2012; U.S. Pat. No. 7,205,275 B2), constrained bicyclic peptides (US 2018/0200378 A1), aptamer, engineered CH2 domains (nanoantibodies; Dimitrov, 2009)) and engineered CH3 domain “Fcab” domains (Wozniak-Knopp et al, 2010).

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof refers to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The terms “Fc receptor” or “FcR,” as used herein, generally refers to a receptor, or any derivative, variant or fragment thereof, that can bind to the Fc region of an antibody. In certain embodiments, the FcR is one which binds an IgG antibody (a gamma receptor, Fcgamma R) and includes receptors of the Fcgamma RI (CD64), Fcgamma RII (CD32), and Fcgamma RIII (CD16) subclasses, including allelic variants and alternatively spliced forms of these receptors. Fcgamma RII receptors include Fcgamma RIIA (an “activating receptor”) and Fcgamma RIB (an “inhibiting receptor”), which have similar amino acid sequences that differ primarily in the cytoplasmic domains thereof. The term “FcR” also includes the neonatal receptor, FcRn, which is responsible for the transfer of maternal IgGs to the fetus.

By “effector function” as used herein is meant a biochemical event that results from the interaction of an antibody Fc region with an Fc receptor or ligand, which vary with the antibody isotype. Effector functions include but are not limited to antibody-dependent cell-mediated cytotoxicity (ADCC), antibody-dependent cell-mediated phagocytosis (ADCP), complement-dependent cytotoxicity (CDC), cytokine secretion, immune complex-mediated antigen uptake by antigen presenting cells, down regulation of cell surface receptors (e.g. B cell receptor), and B cell activation. “Antibody-dependent cell-mediated cytotoxicity” or “ADCC” refers to a cell-mediated reaction in which nonspecific cytotoxic cells that express FcRs (such as Natural Killer (NK) cells, neutrophils, and macrophages) recognize bound antibody on a target cell and subsequently cause lysis of the target cell. ADCC is correlated with binding to FcγRIIIa; increased binding to FcγRIIIa leads to an increase in ADCC activity. To assess ADCC activity of a molecule of interest, an in vitro ADCC assay such as that described in U.S. Pat. No. 5,500,362 or 5,821,337 may be performed. “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

“Fc null” and “Fc null variant” are used interchangeably and used herein to describe a modified Fc which have reduced or abolished effector functions. Such Fc null or Fc null variant have reduced or abolished to FcγRs and/or complement receptors. Preferably, such Fc null or Fc null variant has abolished effector functions. Exemplary methods for the modification include but not limited to chemical alteration, amino acid residue substitution, insertion and deletions. Exemplary amino acid positions on Fc molecules where one or more modifications were introduced to decrease effector function of the resulting variant (numbering based on the EU numbering scheme) at position i) IgG1: C220, C226, C229, E233, L234, L235, G237, P238, S239 D265, S267, N297, L328, P331, K322, A327 and P329, ii) IgG2: V234, G237, D265, H268, N297, V309, A330, A331, K322 and iii) IgG4: L235, G237, D265 and E318. Exemplary Fc molecules having decreased effector function include those having one or more of the following substitutions: i) IgG1: N297A, N297Q, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted; ii) IgG2: A330S/A331S, V234A/G237A, V234A/G237A/D265A, D265A/A330S/A331S, V234A/G237A/D265A/A330S/A331S, and H268Q/V309L/A330S/A331S; iii) IgG4: L235A/G237A/E318A, D265A, L235A/G237A/D265A and L235A/G237A/D265A/E318A.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes may be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

“Linker” as used herein refers to a molecule that connect two polypeptide chains. Linker can be a polypeptide linker or a synthetic chemical linker (for example, see disclosed in Protein Engineering, 9(3), 299-305, 1996). The length and sequence of the polypeptide linkers is not particularly limited and can be selected according to the purpose by those skilled in the art. Polypeptide linker comprises one or more amino acids. Preferably, polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In another embodiment, said peptide linker is (GGGS)xGn (SEQ ID NO:5) or (GGGGS)xGn (SEQ ID NO:6) or (GGGGGS)xGn (SEQ ID NO:7) with x=1, 2, 3, 4, 5, 6, 7, 8, 9, 10, 11, or 12 and n=0, 1, 2 or 3. Preferably, the said linker is (GGGGS)xGn with x=2, 3, or 4 and n=0 (SEQ ID NO:8); more preferably the said linker is (GGGGS)xGn with x=3 and n=0 (SEQ ID NO:9). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl] sulfone (BSOCOES), and bis[2-(succinimidoxycarbonyloxy)ethyl] sulfone (sulfo-BSOCOES).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein, in some examples, refers to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif, FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene, in some cases, refers to an “endogenous gene” or a native gene in its natural location in the genome of an organism. A gene, in some cases, refers to an “exogenous gene” or a non-native gene. A non-native gene, in some cases, refers to a gene not normally found in the host organism but which is introduced into the host organism by gene transfer. A non-native gene, in some cases, refers to a gene not in its natural location in the genome of an organism. A non-native gene, in some cases, also refers to a naturally occurring nucleic acid or polypeptide sequence that comprises mutations, insertions and/or deletions (e.g., non-native sequence).

The term “regulating” with reference to expression or activity, as used herein, refers to altering the level of expression or activity. Regulation can occur at the transcription level and/or translation level.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “residue” as used herein means a position in a protein and its associated amino acid identity. For example, Leu 234 (also referred to as Leu234 or L234) is a residue at position 234 in the human antibody IgG1.

The term “wild-type” as used herein means an amino acid sequence or a nucleotide sequence that is found in nature, including allelic variations. A wild-type protein has an amino acid sequence or a nucleotide sequence that has not been intentionally modified.

The terms “substitution” or “mutation” refers to a change to the polypeptide backbone wherein an amino acid occurring naturally in the wild-type sequence of a polypeptide is substituted to another amino acid not naturally occurring at the same position in the said polypeptide. In some cases, a mutation or mutations is introduced to modify a polypeptide's affinity to its receptor thereby altering its activity such that it becomes different from the affinity and activity of the wild-type cognate polypeptide. Mutations can also improve a polypeptide's biophysical properties. Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

The terms “affinity” or “binding affinity” refer to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity can generally be represented by the dissociation constant (K_{D}), which is the ratio of dissociation and association rate constants (koff and kon, respectively). Thus, equivalent affinities may comprise different rate constants, as long as the ratio of the rate constants remains the same. Affinity can be measured by common methods known in the art, such as enzyme-linked immunosorbent assay (ELISA), surface plasmon resonance (SPR) technologies (e.g., BIAcore), BioLayer Interferometry (BLI) technologies (e.g. Octet) and other traditional binding assays (Heeley, Endocr Res 28, 217-229 (2002).

The terms “binding” or “specific binding” as used here, can refer to the ability of a polypeptide or an antigen binding domain to selectively interact with the receptor for the polypeptide or target antigen, respectively, and this specific interaction can be distinguished from non-targeted or undesired or non-specific interactions. Examples of specific binding can include but are not limited to IL-2 cytokine binding to its specific receptors (e.g., IL-2Rα, IL-2Rβ and IL-2Rγ) and an antigen binding domain binding to a specific antigen (e.g., CD8 or PD-1).

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The terms “effective amount,” or “therapeutically effective amount,” or “effective dose,” or “effective dosage,” refer to the quantity of a composition, for example a composition comprising immune cells such as lymphocytes (e.g., T lymphocytes and/or NK cells), which can be combined with a targeted cytokine construct of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

### Overview

As an overview, the present disclosure relates to methods, compositions, kits, systems, regimens for treatment of a disease or condition, e.g., a proliferative disease, e.g., cancer, by using an engineered cell (e.g., a population of engineered cells for an engineered cell therapy), and a targeted cytokine construct. In some embodiments, provided herein is a combination approach, e.g., administration of both an engineered cell and a targeted cytokine construct, that can improve the therapeutic efficacy of the engineered cell against the disease, e.g., proliferative disease, e.g., cancer. In some aspects, in vitro methods are provided for improving therapeutic efficacy of an engineered cell by contacting a population of the engineered cell with a targeted cytokine construct as described herein.

In some embodiments, the targeted cytokine construct elicits one or more of the following effects in an engineered cell: enhancing the proliferation of such engineered cell; alter cytokine secretion by the engineered cell; decrease the dependence on exogenous cytokines for survival and proliferation of the engineered cell; enhance cytotoxicity of such engineered cell; enhance the survival of such engineered cell; block apoptosis of such engineered cells; delay senescence of such engineered cells; prevent or delay exhaustion of such engineered cells; enhance the persistence of such engineered cells in vivo when administered to a subject; enhance the efficacy of engineered cells in vivo when administered to a subject; enhance the penetration of engineered cells into diseased organs or tissues (e.g., a tumor); or any combinations.

In some embodiments, this disclosure provides methods and compositions for improving in vivo persistence and therapeutic efficacy of engineered cells. In some embodiments, administering a therapeutic regimen comprising a population of the engineered cells and a targeted cytokine construct, improves in vivo persistence of the population of engineered cells by at least about 50%, 60%, 70%, 80%, 90%, 95%, 99%, or 100% as compared to administering the population of engineered cells alone. In some embodiments, administering a therapeutic regimen comprising a population of engineered cells and a targeted cytokine construct, improves in vivo persistence of the population of engineered cells by at least 50%, 60%, 70%, 80%, 90%, 95%, 99%, or 100% as compared to administering the population of engineered cells in combination with an untargeted cytokine or a functional fragment or a variant thereof. In some embodiments, administering a therapeutic regimen comprising a population of engineered cells and a targeted cytokine construct, improves in vivo persistence of the engineered cell by at least 50%, 60%, 70%, 80%, 90%, 95%, 99%, or 100% as compared to administering the population of engineered cells in combination with a cell binding domain that is not fused to a cytokine protein. In some embodiments, a targeted cytokine construct of this disclosure improves the in vitro persistence of a population of engineered cells by at least about 10%, 20%, 30%, 40%, 50%, 60%, 70%, 80%, 90%, 95%, 99%, or 100%, when the population of engineered cells is contacted with the targeted cytokine construct.

In some embodiments, this disclosure provides methods and compositions for improving in vivo persistence and therapeutic efficacy of engineered cells. In some embodiments, administering a therapeutic regimen comprising a population of the engineered cells and a targeted cytokine construct, improves in vivo persistence of the population of engineered cells by at least about 2 fold as compared to administering the population of engineered cells alone. In some embodiments, administering a therapeutic regimen comprising a population of engineered cells and a targeted cytokine construct, improves in vivo persistence of the population of engineered cells by at least 2 fold as compared to administering the population of engineered cells in combination with an untargeted cytokine or a functional fragment or a variant thereof. In some embodiments, administering a therapeutic regimen comprising a population of engineered cells and a targeted cytokine construct, improves in vivo persistence of the engineered cell by at least 2 fold as compared to administering the population of engineered cells in combination with a cell binding domain that is not fused to a cytokine protein.

In some embodiments, the present disclosure relates to a method of reducing an effective dosage of an engineered cell in an engineered cell therapy comprising administering a population of the engineered cell and a targeted cytokine construct as described herein. In some embodiments, a composition comprising a population of engineered cells and a targeted cytokine construct comprises a lower amount of the engineered cell, compared to the amount in a reference composition comprising the population of the engineered cell but not the targeted cytokine construct, wherein both compositions exhibit similar efficacy.

In some embodiments, a composition comprising a population of engineered cells and a targeted cytokine construct comprises a lower amount of the engineered cell, compared to the amount in a reference composition comprising the population of engineered cells and an untargeted cytokine or a functional fragment or a variant thereof. In some embodiments, a composition comprising a population of engineered cells and a targeted cytokine construct contains a lower amount of an engineered cell, compared to the amount in a reference composition comprising the population of engineered cells and the cell binding domain, but not the cytokine protein. In some embodiments, the effective dose or the amount, as described above, is lower in the composition by about 1.5× to about 1000×, such as about 2×, about 4×, about 8×, about 16×, about 32×, about 50×, about 64×, about 70×, about 75×, about 80×, about 90×, about 91×, about 92×, about 93×, about 94×, about 95×, about 96×, about 97×, about 98×, about 99×, or about 1000×, relative to that in the reference composition.

Studies have shown that loss of CAR T cells is associated with either T cell-intrinsic issues preventing expansion or T cell-extrinsic immunological rejection. Patients who experience a relapse after CAR T cell-induced remission have very early loss of CAR T cells or loss of B-cell aplasia (absolute B cells typically measured by flow rising above 50 to 100/μL)—which loss has generally been associated with relapses (e.g., CD19+ B-cell relapses). See, e.g., Nie et al. “Mechanisms underlying CD19-positive ALL relapse after anti-CD19 CAR T cell therapy and associated strategies,” Biomarker Research volume 8, Article number: 18 (2020). In patients who have relapsed after treatments with engineered cells (e.g., CAR-T cell therapy, such as s CD19 CAR-T cell therapy), administering a targeted cytokine construct of this disclosure can function as a salvage therapy. Thus, in some embodiments of this disclosure are provide methods of treatment by administering a targeted cytokine construct, to patients who have undergone a loss of B cell aplasia, following an engineered cell therapy.

In some embodiments is provided a therapeutic regimen comprising administering to a subject a population of engineered cells and a targeted cytokine construct as described herein, and the method allows for at least one of: (a) avoiding a prior administering of a lymphodepleting agent to the subject, or (b) reduces the extent of prior lymphodepletion required for expansion, engraftment of the engineered cell. Non-limiting examples of lymphodepleting agents include, chemotherapeutic agents such as fludarabine, cyclophosphamide, and depleting antibody such as alemtuzumab.

In some embodiments, the targeted cytokine construct is administered to target engineered cells that have been generated in vivo in a subject. Various improvements to such in vivo generated engineered cells can be realized by administering the targeted cytokine construct, e.g., increased persistence, reduced rate and/or extent of exhaustion, increased expansion and/or proliferation, no increase in Treg cell count, selective potentiation and specific enrichment of the engineered cells. Example techniques for generating the engineered cells (e.g., CAR-T or TCR-T cells) in vivo, include, administering to a subject a nucleic acid carrier that includes CAR or TCR genes. In some embodiments, nucleic acid carrier is a non-viral vector, a plasmid, a linear polynucleotide, a polynucleotide associated with ionic or amphiphilic compounds, a plasmid, or a virus (such as a viral vector). In some embodiments, a viral vector is at least one of: a Sendai viral vector, an adenoviral vector, an adeno-associated virus vectors, a retroviral vector, or a lentiviral vector. The nucleic acid carriers, in some cases, comprise a targeting moiety specific for an immune cell (e.g., a T cell, an NK cell, a T lymphocyte, a myeloid cell). The immune cells targeted by the nucleic acid carriers, in some embodiments, are induced to generate the in vivo engineered cells, which are targeted by the targeted cytokine constructs of this disclosure.

### Combination Therapy

In one aspect, the present disclosure provides a combination therapy, e.g., therapeutic methods and regimens, for treating a disease or disorder, e.g., a cancer or proliferative disease, that includes administering to a subject a therapeutic regimen comprising of (1) an engineered cell, e.g., CAR-expressing cell, e.g., T cells, a population thereof, and (2) a targeted cytokine construct. In another aspect, the present disclosure provides a method of improving in vitro activation and/or expansion of a population engineered cells, by contacting the population of engineered cells with a targeted cytokine construct as described herein, and in some embodiments, is further provided an engineered cell therapy which comprises administering the population of engineered cells, for treating a disease or disorder, e.g., a cancer or proliferative disease. The administering, as described above, can be in combination with a targeted cytokine construct of this disclosure.

In some embodiments, the engineered cells specifically recognize and/or target an antigen associated with a disease or disorder, e.g., a cancer or proliferative disease. In some embodiments, the engineered cells express a domain or a receptor that is recognized by the cytokine of the targeted cytokine construct. Also provided are combinations and articles of manufacture, such as kits, that contain a composition comprising a population of engineered cells and/or a composition comprising the targeted cytokine construct, and uses of such compositions and combinations to treat diseases, conditions, and disorders, including cancers. Such methods can include administration of the targeted cytokine construct prior to, simultaneously with, during, during the course of (including once and/or periodically during the course of), and/or subsequently to, the administration (e.g., initiation of administration) of a therapy comprising the engineered cells (e.g., CAR-expressing T cells). In some embodiments, the administrations can involve sequential or intermittent administrations of the targeted cytokine construct and/or a population of the engineered cells (administering a population of engineered cells is also referred to herein as an engineered cell therapy).

### Administration Dosages and Regimen

In some embodiments, the present disclosure provides a therapeutic regiment that comprises a first pharmaceutical composition that comprises a population of engineered cells; and a second pharmaceutical composition that comprises a targeted cytokine construct. In some embodiments, the second pharmaceutical composition comprises the targeted cytokine construct in an amount sufficient to enhance the therapeutic effects of the population of engineered cells.

In some embodiments, the first composition comprising the population of engineered cells and the second pharmaceutical composition comprising the targeted cytokine construct are administered to a subject simultaneously or sequentially. In some embodiments, a subject receives administration of a therapy comprising a population of engineered cells first, for instance, finishes the therapy comprising the population of engineered cells first, and then receives administration of a targeted cytokine construct.

In some embodiments, the therapy comprising the population of engineered cells and the targeted cytokine construct are administered to a subject according to a pre-determined regimen, e.g., concurrently, intermittently, or sequentially. In some embodiments, the subject receives administration of the therapy comprising the population of engineered cells according to a prescribed administration regimen, e.g., once, twice, 3, 4, 5, 6, or 7 times a week for a number of consecutive weeks, or once every 1, 2, 3, 4, 5, 6, or 7 days for a given period of time, e.g., a week, a month, or a year. In some embodiments, the subject also receives administration of the targeted cytokine construct according to the same administration regimen as the therapy comprising the population of engineered cells, or in some cases, according to an administration regimen that overlaps with the administration regimen of the therapy comprising the population of engineered cells. For example, a subject can be taking both the therapy comprising the population of engineered cells, e.g., a T cell infusion, and the targeted cytokine construct, at the same time, for instance, both via intravenous infusion. In other cases, a subject can be taking both the therapy comprising the population of engineered cells, e.g., a T cell infusion, and the targeted cytokine construct, on a same day. In some cases, when the two administration regimens overlap, a subject can be taking the therapy comprising the population of engineered cells on one day, and then taking the targeted cytokine construct on the next day, or 2, 3, 4, 5, 6, 7, or more days after and before receiving the next administration of the therapy comprising the population of engineered cells. Alternatively, in some cases, a subject can be administered with the therapy comprising the population of engineered cells more times as compared to the targeted cytokine construct, or vice versa.

In some embodiments, the therapy comprising the population of engineered cells is administered concurrently with or after starting or initiating administration of the targeted cytokine construct. In some embodiments, the therapy comprising the population of engineered cells is administered 0 to 90 days, such as 0 to 30 days, 0 to 15 days, 0 to 6 days, 0 to 96 hours, 0 to 24 hours, 0 to 12 hours, 0 to 6 hours, or 0 to 2 hours, 2 hours to 30 days, 2 hours to 15 days, 2 hours to 6 days, 2 hours to 96 hours, 2 hours to 24 hours, 2 hours to 12 hours, 2 hours to 6 hours, 6 hours to 90 days, 6 hours to 30 days, 6 hours to 15 days, 6 hours to 6 days, 6 hours to 96 hours, 6 hours to 24 hours, 6 hours to 12 hours, 12 hours to 90 days, 12 hours to 30 days, 12 hours to 15 days, 12 hours to 6 days, 12 hours to 96 hours, 12 hours to 24 hours, 24 hours to 90 days, 24 hours to 30 days, 24 hours to 15 days, 24 hours to 6 days, 24 hours to 96 hours, 96 hours to 90 days, 96 hours to 30 days, 96 hours to 15 days, 96 hours to 6 days, 6 days to 90 days, 6 days to 30 days, 6 days to 15 days, 15 days to 90 days, 15 days to 30 days or 30 days to 90 days after starting or initiating administration of the targeted cytokine construct. In some embodiments, the therapy comprising the population of engineered cells is administered at least or about at least or about 1 hour, 2 hours, 6 hours, 12 hours, 24 hours, 2 days, 3 days, 6 days, 12 days, 15 days, 30 days, 60 days or 90 days after starting or initiating administration of the targeted cytokine construct.

In some embodiments, the therapy comprising the population of engineered cells is administered concurrently with or before starting or initiating administration of targeted cytokine construct. In some embodiments, the therapy comprising the population of engineered cells is administered 0 to 90 days, such as 0 to 30 days, 0 to 15 days, 0 to 6 days, 0 to 96 hours, 0 to 24 hours, 0 to 12 hours, 0 to 6 hours, or 0 to 2 hours, 2 hours to 30 days, 2 hours to 15 days, 2 hours to 6 days, 2 hours to 96 hours, 2 hours to 24 hours, 2 hours to 12 hours, 2 hours to 6 hours, 6 hours to 90 days, 6 hours to 30 days, 6 hours to 15 days, 6 hours to 6 days, 6 hours to 96 hours, 6 hours to 24 hours, 6 hours to 12 hours, 12 hours to 90 days, 12 hours to 30 days, 12 hours to 15 days, 12 hours to 6 days, 12 hours to 96 hours, 12 hours to 24 hours, 24 hours to 90 days, 24 hours to 30 days, 24 hours to 15 days, 24 hours to 6 days, 24 hours to 96 hours, 96 hours to 90 days, 96 hours to 30 days, 96 hours to 15 days, 96 hours to 6 days, 6 days to 90 days, 6 days to 30 days, 6 days to 15 days, 15 days to 90 days, 15 days to 30 days or 30 days to 90 days before starting or initiating administration of the targeted cytokine construct. In some embodiments, the therapy comprising the population of engineered cells is administered at least or about at least or about 1 hour, 2 hours, 6 hours, 12 hours, 24 hours, 2 days, 3 days, 6 days, 12 days, 15 days, 30 days, 60 days or 90 days before starting or initiating administration of the targeted cytokine construct.

In some embodiments, following an initial administering of a targeted cytokine construct and a population of engineered cells, subsequent doses of the targeted cytokine construct is administered to the subject, for maintenance of the engineered cells in vivo. The subsequent administrations, in some embodiments, are once every one week, once every two weeks, once every three weeks, or monthly, after the initial administration.

In some embodiments, the dosage and timing of the targeted cytokine construct is adjusted based on the measurement of one or more therapeutic effects associated with the administration of the therapy comprising the population of engineered cells, in a sample from the subject after the administration of the therapy comprising the population of engineered cells. In some cases, the administration regimen of the composition comprising the population of engineered cells and the composition comprising targeted cytokine construct is determined by an attending physician of the subject. The physician's decision can be based on a number of factors, including, but not limited to, medical history of the subject and other medical exam results of the subject, e.g., pathological exam results of the tumor. The specific dose of the targeted cytokine construct will vary depending on the particular combination of cytokine and cell binding domain chosen, the dosing regimen to be followed, the health condition of the subject, the tissue to which it is administered, and the physical delivery system in which it is carried. In some embodiments, a targeted cytokine construct is administered to a subject within a range of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 150, 200, 300, 400, 500, 600, 700, 800, 900, or 1000 mg per week on average over the course of a treatment cycle. For example, the targeted cytokine construct is administered to a subject within a range of about 35, 36, 37, 38, 39, 40, 41, 42, 43, 44, 45, 46, 47, 48, 49, 50, 51, 52, 53, 54, or 55 mg per week. In some embodiments, the targeted cytokine construct is administered to a subject within a range of about 40, 41, 42, 43, 44, 45, 46, 47, 48, 49, 50, 51, 52, 53, 54, or 55 mg per week.

In some embodiments, a targeted cytokine construct is administered to a subject in an amount greater than 1, 1.5, 2, 2.5, 3, 3.5, 4, 4.5, 5, 5.5, 6, 6.5, 7, 7.5, 8, 8.5, 9, 9.5, or 10 mg per day on average over the course of a treatment cycle. For example, the targeted cytokine construct is administered to a subject in an amount between about 6 and 10 mg, between about 6.5 and 9.5 mg, between about 6.5 and 8.5 mg, between about 6.5 and 8 mg, or between about 7 and 9 mg per day on average over the course of a treatment cycle.

In some embodiments, a targeted cytokine construct is administered to a subject within a range of about 0.01 mg/kg-50 mg/kg per day, such as about, less than about, or more than about, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, or 50 mg/kg per day. In some embodiments, a targeted cytokine construct is administered to a subject within a range of about 0.1 mg/kg-400 mg/kg per week, such as about, less than about, or more than about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 100 mg/kg, 150 mg/kg, 200 mg/kg, 250 mg/kg, 300 mg/kg, 350 mg/kg, or 400 mg/kg per week. In some embodiments, a targeted cytokine construct and administered to a subject at a dose of about 1 mg/kg weekly or bi-weekly.

In some embodiments, a targeted cytokine construct is administered to a subject within a range of about 0.4 mg/kg-1500 mg/kg per month, such as about, less than about, or more than about 0.4 mg/kg, 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg per month. In some embodiments, a targeted cytokine construct is administered to a subject within a range of about 0.1 mg/m²-200 mg/m² per week, such as about, less than about, or more than about 1 mg/m², 5 mg/m², 10 mg/m², 15 mg/m², 20 mg/m², 25 mg/m², 30 mg/m², 35 mg/m², 40 mg/m2, 45 mg/m², 50 mg/m², 55 mg/m², 60 mg/m², 65 mg/m², 70 mg/m², 75 mg/m², 100 mg/m², 125 mg/m2, 150 mg/m², 175 mg/m², or 200 mg/m2 per week. The target dose may be administered in a single dose. Alternatively, the target dose may be administered in about or more than about 1, 2, 3, 4, 5, 6, 7, 8, 9, 10, 11, 12, 13, 14, 15, 16, 17, 18, 19, 20, 25, 30, or more doses. For example, a dose of about 1 mg/kg per week may be delivered weekly at a dose of about 1 mg/kg every week, about 2 mg/kg administered every two weeks, or about 4 mg/kg administered every four weeks over the course of the week. The administration schedule may be repeated according to any regimen as described herein, including any administration schedule described herein. In some embodiments, a targeted cytokine construct is administered to a subject in the range of about 0.1 mg/m²-500 mg/m², such as about, less than about, or more than about 1 mg/m², 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 100 mg/m², 130 mg/m², 135 mg/m2, 155 mg/m², 175 mg/m², 200 mg/m², 225 mg/m², 250 mg/m², 300 mg/m², 350 mg/m², 400 mg/m², 420 mg/m², 450 mg/m², or 500 mg/m².

In some embodiments, a dose of the targeted cytokine construct is about, at least about, or at most about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 mg or mg/kg, or any range derivable therein. It is contemplated that a dosage of mg/kg refers to the mg amount of the targeted cytokine construct per kg of total body weight of the subject. It is contemplated that when multiple doses are given to a patient, the doses may vary in amount or they may be the same.

### Therapeutic Effects

In some embodiments, the methods described herein enable modulation of the rate of in vivo expansion and/or proliferation of a population of engineered cells (an engineered cell therapy) administered to a subject, or an in vitro proliferation of a population of engineered cells. In some embodiments, the treatment methods described herein increase expansion and/or proliferation of administered engineered cells in vivo relative to engineered cells administered to a subject in the absence of a targeted cytokine construct. In some embodiments, the treatment methods described herein increase expansion and/or proliferation of administered engineered cells in vivo by at least 2, 3, 4, 5, 6, 7, 8, 9, 10, 20, 30, 40, 50, 60, 70, 80, 90, or 100 fold, relative to engineered cells administered to a subject in the absence of a targeted cytokine construct. In some embodiments, the targeted cytokine constructs of this disclosure increase expansion and/or proliferation of engineered cells in vitro relative to engineered cells that are not contacted with a targeted cytokine construct as described herein, by at least about 2, 3, 4, 5, 6, 7, 8, 9, 10, 20, 30, 40, 50, 60, 70, 80, 90, or 100 fold. In some cases, the increased expansion and/or proliferation of a population of engineered cells, in vivo, is at least about 2, 3, 4, 5, 6, 7, 8, 9, 10, 20, 30, 40, 50, 60, 70, 80, 90, or 100 fold, when the population of engineered cell is administered in combination with the targeted cytokine construct, is relative to administering the engineered cells in combination with at least one of: (a) a cell binding domain without the cytokine; (b) an untargeted cytokine or a functional fragment or a variant thereof, without the cell binding domain. Similarly, in some cases, the increased in vitro expansion and/or proliferation of a population of engineered cells when contacted with a targeted cytokine construct, by at least about 2, 3, 4, 5, 6, 7, 8, 9, 10, 20, 30, 40, 50, 60, 70, 80, 90, or 100 fold, is relative to contacting the population of engineered cells with at least one of: (a) a cell binding domain without the cytokine; (b) an untargeted cytokine or a functional fragment or a variant thereof, without the cell binding domain.

In some embodiments, the methods described herein enable enhanced specific enrichment of a population of the engineered cell administered to a subject, relative to administering the populating of engineered cells alone or in combination with an untargeted cytokine or a functional fragment or a variant thereof, without the cell binding domain. In some embodiments, the engineered cells express a receptor for the cytokine protein and the methods describe herein result in selective outgrowth of the engineered cells (e.g., cells expressing CAR), as opposed to outgrowth of cells that do not express the receptor for the CAR. In some embodiments, the methods described herein results in no increase of Treg cells, following administering a population of engineered cells in combination with a targeted cytokine construct, in contrast to increase in Treg cells upon administering the population of engineered cells alone or in combination with an untargeted cytokine or a functional fragment or a variant thereof, without the cell binding domain. In some embodiments, the methods described herein reduces the rate and/or extent of exhaustion of a population of the engineered cell administered to a subject, relative to administering the population of engineered cells alone or in combination with an untargeted cytokine or a functional fragment or a variant thereof, without the cell binding domain, or in combination with the cell binding domain without the cytokine.

In some embodiments, the methods described herein increase in vivo persistence of a population of the engineered cell administered to a subject, relative to administering the population of engineered cells without the targeted cytokine construct or when administered with the cell binding domain without the cytokine. In some embodiments, the methods described herein increase in vitro persistence of a population of the engineered cells when contacted with a targeted cytokine construct as described herein, relative to in vitro persistence of the population of engineered cells when not contacted with the targeted cytokine construct or when contacted with the cell binding domain without the cytokine. In some embodiments, the in vivo or in vitro persistence increase is at least about 2, 3, 4, 5, 6, 7, 8, 9, 10, 20, 30, 40, 50, 60, 70, 80, 90, or 100 fold greater, than the comparators described above. In some examples, the in vivo persistence of the population of engineered cells when administered to a subject in combination with the targeted cytokine construct is at least about 30 days to about an year or longer, such as, about 45 days, about 60 days, about 90 days, about 120 days, about 6 months, about 7 months, about 8 months, about 9 months, about 10 months, about 11 months, about 12 months, about 18 months or longer.

The methods, compositions, combinations, kits and regimens provided herein can in some aspects allow for a reduction in the severity of a preconditioning regimen (e.g., a lymphodepletion regimen) needed for effective engraftment of engineered cells. Thus, in one instance, prior to administering (e.g., infusing) a population of engineered cells, a subject is lymphodepleted. In other instances, lymphodepletion is not required and the population of engineered cells are rapidly infused into the subject.

In some embodiments, the methods include administering a preconditioning agent, such as a lymphodepleting or chemotherapeutic agent, such as cyclophosphamide, fludarabine, or combinations thereof, or an antibody, such as alemtuzumab, which is a depleting antibody, to a subject prior to the initiation of the engineered cell therapy. In some embodiments, the methods, compositions, combinations, kits and regimens provided herein can eliminate the need for administering a preconditioning agent/regimen (e.g., lymphodepleting agent/regimen), to a subject prior to administering an engineered cell therapy, in combination with a targeted cytokine construct.

In subjects who are administered a preconditioning agent, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with, for example, cyclophosphamide at a dose that is lower than 100 mg/kg body weight of the subject, such as lower than about 20 mg/kg, lower than about 40 mg/kg, or lower than about 80 mg/kg. The preconditioning agent is thus administered at a dosage that is lower for when the engineered cell therapy is administered in combination with a targeted cytokine construct, relative to when the engineered cell therapy is administered alone, or in combination with at least one of: (a) a cell binding domain without the cytokine, or (b) an untargeted cytokine or a functional fragment or a variant thereof, without the cell binding domain. In some aspects, the subject is preconditioned or administered with a preconditioning agent, such as cyclophosphamide, in a single dose or in a plurality of doses, such as given daily, every other day or every three days. In some embodiments, a preconditioning agent, such as cyclophosphamide, is administered to the subject at a dose less than about 500 mg/m2 body surface area of the subject, such as less than about 400 mg/m², less than about 300 mg/m², less than about 250 mg/m², less than about 200 mg/m², or less than about 100 mg/m2.

Exemplary lymphodepletion regimens are listed in the tables below:

TABLE 1
Example lymphodepletion regimen

| | |
|---|---|
| Infusion D-6 | Admit/IV hydration |
| Infusion D-5 | Fludarabine 25 mg/m2, Cyclophosphamide |
| | 250 mg/m2 |
| Infusion D-4 | Fludarabine 25 mg/m2, Cyclophosphamide |
| | 250 mg/m2 |
| Infusion D-3 | Fludarabine 25 mg/m2 IV, |
| | Cyclophosphamide 250 mg/m2 |
| Infusion D-2 | Rest |
| Infusion D-1 | Rest |
| Infusion D (DO) | Engineered cell infusion (e.g., CAR + T cell |
| | infusion) |

TABLE 2
Example lymphodepletion regimen

| | |
|---|---|
| Infusion D-6 | Admit/IV hydration |
| Infusion D-5 | Fludarabine 30 mg/m2, Cyclophosphamide |
| | 500 mg/m2 |
| Infusion D-4 | Fludarabine 30 mg/m2, Cyclophosphamide |
| | 500 mg/m2 |
| Infusion D-3 | Fludarabine 30 mg/m2 IV, |
| | Cyclophosphamide 500 mg/m2 |
| Infusion D-2 | Rest |
| Infusion D-1 | Rest |
| Infusion D (DO) | Engineered cell infusion (e.g., CAR + T cell |
| | infusion) |

The methods, compositions, combinations, kits and regimens provided herein can treat proliferative diseases, such as cancer. In some embodiments, the combination therapy provided herein have advantageous therapeutic effects in treating the proliferative diseases, such as cancer.

In some embodiments of the methods, compositions, combinations, kits and uses provided herein, the provided combination therapy results in one or more treatment outcomes, such as a feature associated with any one or more of the parameters associated with the therapy or treatment, as described below.

In some embodiments, parameters associated with therapy or a treatment outcome, which include parameters that can be assessed for the screening steps and/or assessment of treatment of outcomes and/or monitoring treatment outcomes, includes tumor or disease burden. The administration of a therapy comprising the engineered cells, such as a T cell therapy (e.g., CAR-expressing T cells) and/or a targeted cytokine construct as described here, can reduce or prevent the expansion or burden of the disease or condition in the subject. For example, where the disease or condition is a tumor, the methods can generally reduce tumor size, bulk, metastasis, percentage of blasts in the bone marrow or molecularly detectable cancer and/or improve prognosis or survival or other symptom associated with tumor burden.

In some embodiments, the provided combination therapy results in a decreased tumor burden in treated subjects compared to alternative methods in which the engineered cell therapy (e.g., CAR-expressing T cells) is given without administration of the targeted cytokine construct. It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In some embodiments, the subject has a myeloma, a lymphoma or a leukemia. In some embodiments, the subject has a non-Hodgkin lymphoma (NHL), an acute lymphoblastic leukemia (ALL), a chronic lymphocytic leukemia (CLL), a diffuse large B-cell lymphoma (DLBCL) or a myeloma, e.g., a multiple myeloma (MM). In some embodiments, the subject has a MM or a DBCBL. In some embodiments, the subject has a solid tumor.

In the case of multiple myeloma, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/1); renal insufficiency attributable to myeloma; anemia (hemoglobin <10 g/dl); and/or bone lesions (lytic lesions or osteoporosis with compression fractures)). In the case of diffuse large B-cell lymphoma, exemplary parameters to assess the extent of disease burden include such parameters as cellular morphology (e.g., centroblastic, immunoblastic, and anaplastic cells), gene expression, miRNA expression and protein expression (e.g., expression of BCL2, BCL6, MUM1, LM02, MYC, and p21). In the case of leukemia, the extent of disease burden can be determined by assessment of residual leukemia in blood or bone marrow. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow. In some embodiments, for leukemia, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or the subject exhibits minimal residual disease (MRD), such that, in some cases, no leukemia cells are able to be detected in the subject using PCR or flow cytometry techniques.

In some embodiments, the combination therapy provided herein decreases disease burden as compared with disease burden at a time immediately prior to the administration of the combination therapy. In some aspects, administration of the combination therapy prevents an increase in disease burden, and this may be evidenced by no change in disease burden. In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives the engineered cell therapy alone, in the absence of administration of the targeted cytokine construct. In some embodiments, disease burden is reduced to a greater extent or for a greater duration following the combination therapy of administration of the engineered cell therapy, and the targeted cytokine construct, compared to the reduction that would be effected by administering each of the agent alone, e.g., administering the targeted cytokine construct to a subject having not received the engineered cell therapy; or administering the engineered cell therapy, to a subject having not received the targeted cytokine construct.

In some embodiments, the burden of a disease or condition in the subject is detected, assessed, or measured. Disease burden can be detected in some aspects by detecting the total number of disease or disease-associated cells, e.g., tumor cells, in the subject, or in an organ, tissue, or bodily fluid of the subject, such as blood or serum. In some embodiments, disease burden, e.g., tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some aspects, disease burden is measured or detected prior to administration of the engineered cell therapy, following the administration of the engineered cell therapy but prior to administration of the targeted cytokine construct, or following administration of the targeted cytokine construct but prior to the administration of the engineered cell therapy, and/or following the administration of both the engineered cell therapy and the targeted cytokine construct. In the context of multiple administration of one or more steps of the combination therapy, disease burden in some embodiments may be measured prior to or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100 percent by the provided methods compared to immediately prior to the administration of the targeted cytokine construct and the engineered cell therapy. In some embodiments, disease burden, tumor size, tumor volume, tumor mass, and/or tumor load or bulk is reduced following administration of the engineered cell therapy, and the targeted cytokine construct, by at least at or about 10, 20, 30, 40, 50, 60, 70, 80, 90% or more compared to that immediately prior to the administration of the engineered cell therapy and/or the targeted cytokine construct.

In some embodiments, reduction of disease burden by the method comprises an induction in morphologic complete remission, for example, as assessed at 1 month, 2 months, 3 months, or more than 3 months, after administration of, e.g., initiation of, the combination therapy. In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some embodiments, engineered cells described herein are modified (e.g., contacted with a targeted cytokine construct of the disclosure) at a point-of-care site. In some cases, the point-of-care site is at a hospital or at a facility (e.g., a medical facility) near a subject in need of treatment. The subject undergoes apheresis and peripheral blood mononuclear cells (PBMCs) obtained can be enriched for example, by elutriation. In one instance, the elutriation process is performed using a buffer solution containing human serum albumin. Engineered cells, e.g., CAR+ T cells, TCR transduced T cells, can be isolated by selection methods described herein. In one instance, the selection method for engineered cells includes beads specific for markers such as CD3 and CD8 on the engineered cells (e.g., CAR+ T cells). In one case, the beads can be paramagnetic beads. Harvested and modified cells can be cryopreserved in any appropriate cryopreservation solution prior to modification. Cells can be thawed up to 24 hours, 36 hours, 48 hours. 72 hours or 96 hours ahead of infusion. The thawed cells can be placed in cell culture buffer, for example in cell culture buffer (e.g., RPMI) supplemented with fetal bovine serum (FBS) or placed in a buffer that includes IL-2 and IL-21, prior to a modification. In another aspect, harvested cells can be modified immediately without the need for cryopreservation.

In some cases, the harvested cells are modified by engineering/introducing a chimeric receptor, one or more cell tag(s), and/or contacting with a targeted cytokine construct of this disclosure and then rapidly infused into a subject. In some cases, the sources of cells can include both allogeneic and autologous sources. In one case, the cells can be T cells or NK cells. In one case, the chimeric receptor can be a CAR or a TCR. In some cases, the cells are modified by contacting with a targeted cytokine construct comprising a cytokine protein of functional fragment or variant comprising a sequence selected from the group consisting of SEQ ID NOs: 11-90, or a sequence that is at least about 75% to about 99% identical to a sequence selected from the group consisting of SEQ ID NOs: 11-90.

In certain cases, the engineered cells are modified by expressing a cell tag on cell surface that is separate from CAR or TCR molecules, such as a truncated epidermal growth factor, EGFRt. In some instances, the cell tag is activated, for example via cetuximab, for conditional in vivo ablation of modified engineered cells comprising cell tags such as truncated epidermal growth factor receptor tags as described herein. In some embodiments, the cells are modified by expressing a chimeric antigen receptor or a T cell receptor, or portions thereof, that comprise a polypeptide tag sequence such as a myc tag with the following sequence: EQKLISEEDL. The targeted cytokine constructs of this disclosure, in some instances, comprise a cell binding domain (such as an antibody or an antigen binding fragment thereof) that is specific for such tags as described above, expressed by a cell or expressed as part of a CAR or a TCR molecule that is expressed by the cell. Examples are anti-EGFR antibodies that can be used to bind to EGFRt tag expressed on the engineered cells, or anti-myc antibodies that can be used to bind to the myc tag expressed as part of the CAR or TCR molecules on the engineered cell.

In some embodiments, harvested cells are modified by targeted cytokine constructs through electroporation. In one instance, electroporation is performed with electroporators such as Lonza's Nucleofector™ electroporators. In other embodiments, the vector comprising the above-mentioned constructs is a non-viral or viral vector. In one case, the non-viral vector includes a Sleeping Beauty transposon-transposase system. In one instance, the cells are electroporated using a specific sequence. For example, the cells can be electroporated with one transposon followed by the DNA encoding the transposase followed by a second transposon. In another instance, the immune effector cells can be electroporated with all transposons and transposase at the same time. In another instance, the cells can be electroporated with a transposase followed by both transposons or one transposon at a time. While undergoing sequential electroporation, the cells may be rested for a period of time prior to the next electroporation step.

In some cases, the modified cells do not undergo a propagation and activation step. In some cases, the modified cells do not undergo an incubation or culturing step (e.g., ex vivo propagation). In other instances, the modified immune effector cells are placed or rested in cell culture buffer, for example in cell culture buffer (e.g., RPMI) supplemented with fetal bovine serum (FBS) prior to infusion. Prior to infusion, the modified cells can be harvested, washed and formulated in saline buffer in preparation for infusion into the subject.

### Targeted Cytokine Constructs

The present disclosure provides, in some embodiments, a targeted cytokine construct comprising: a cell binding domain that targets at least one of: (i) a domain of a chimeric antigen receptor (CAR) or a T cell receptor (TCR) exogenously introduced into the engineered cell; (ii) a tag molecule selectively expressed on the surface of the engineered cell; (iii) a polypeptide tag that is part of a CAR exogenously introduced into the engineered cell; (iv) a polypeptide tag that is part of the TCR, or (vi) any combination of (i)-(v), and—a cytokine protein or a functional fragment or a variant thereof.

One embodiment provides a targeted cytokine construct for use in a combination therapy with an engineered cell, the fusion protein comprising (i) a cell binding domain, and (ii) a cytokine protein or a functional fragment or a variant thereof, wherein the cell binding domain: (a) comprises an antibody or an antigen binding fragment thereof that is specific for a domain of an antigen receptor expressed on the engineered cell (e.g., a CAR or TCR); (c) is specific for a tag, wherein the tag is a surface molecule co-expressed by the engineered cell (“separately expressed”) or is part of the antigen receptor (e.g., CAR or TCR) expressed by the engineered cell (“part of CAR” or “part of TCR”); (d) is a domain from an antigen targeted by the engineered cell; or (e) comprises any combinations of (a)-(d). In some embodiments, the receptor expressed by the engineered cell is a chimeric antigen receptor (CAR) or a T cell receptor (TCR).

In some embodiments, targeted cytokine construct comprises two moieties, wherein: i) The first moiety is a polypeptide comprising an antibody heavy chain VH-CH1-hinge-CH2-CH3 monomer wherein VH is a variable heavy chain and CH2-CH3 is a Fc domain, an antibody light chain VL-CL wherein VL is a variable light chain and CL is a constant light chain, and a mutant cytokine polypeptide, wherein the N-terminus of the mutant cytokine polypeptide is fused to the C-terminus of the Fc domain via a linker; ii) The second moiety is a polypeptide comprising an antibody heavy chain VH-CH1-hinge-CH2-CH3 monomer and an antibody light chain VL-CL; and wherein, both the first and second moiety bind to a domain selectively expressed on engineered cells over non-engineered cells.

In some embodiments, targeted cytokine construct comprises two moieties, wherein: i) The first moiety is a polypeptide comprising an antibody hinge-CH2-CH3 monomer wherein CH2-CH3 is a Fc domain, and a mutant cytokine polypeptide, wherein the N-terminus of the mutant cytokine polypeptide is fused to the C-terminus end of the Fc domain via a linker; ii) The second moiety is a polypeptide comprising an antibody heavy chain VH-CH1-hinge-CH2-CH3 monomer and an antibody light chain VL-CL; and wherein the second moiety binds to a domain selectively expressed on engineered cells over non-engineered cells.

In some embodiments, targeted cytokine construct comprises two moieties, wherein: i) The first moiety is a polypeptide comprising an antibody hinge-CH2-CH3 monomer wherein CH2-CH3 is a Fc domain, and a mutant cytokine polypeptide, wherein the C-terminus of the mutant cytokine polypeptide is fused to the N-terminus end of the Fc domain via a linker; ii) The second moiety is a polypeptide comprising an antibody heavy chain VH-CH1-hinge-CH2-CH3 monomer and an antibody light chain VL-CL; and wherein the second moiety binds to a domain selectively expressed on engineered cells over non-engineered cells

The present disclosure provides, in some embodiments, a targeted cytokine construct comprising a (i) a cell binding domain, and (ii) a cytokine protein or a functional fragment or a variant thereof, wherein the cell binding domain: (a) comprises an antibody or an antigen binding fragment thereof that is specific for a receptor or domain exogenously expressed on the surface of the engineered cell; (b) comprises an antibody or an antigen binding fragment thereof that is specific for a domain of an antigen binding protein expressed on the engineered cell; (c) is specific for a tag co-expressed by the engineered cell, or a tag on a receptor (e.g., a chimeric antigen receptor—CAR; or a T cell receptor—TCR) expressed by the engineered cell; (d) is a domain from an antigen targeted by the engineered cell; or (e) comprises any combinations of (a)-(d).

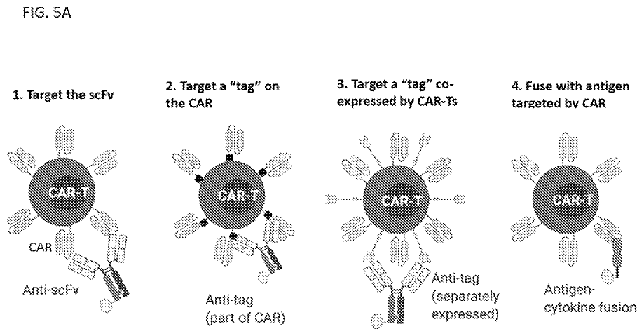

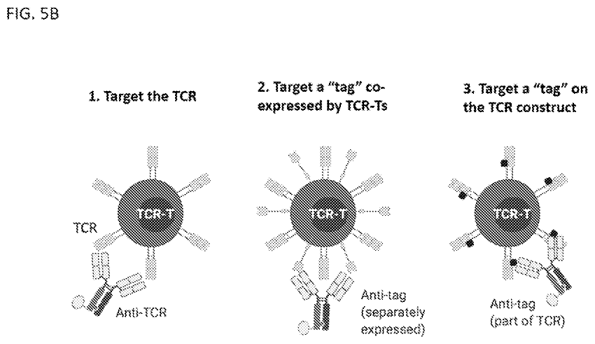

The cell binding domain, in some examples, is an antibody or an antigen binding fragment thereof that is specific for a receptor or domain exogenously expressed on the surface of the engineered cell. In some embodiments, the cell binding domain comprises an anti-idiotype antibody specific to the engineered cell. In some embodiments, the cell binding domain is specific for a domain of an antigen binding protein expressed on the engineered cell, for instance, an scFv expressed on the engineered cell where the cell binding domain is specific for the VH-VL interface, VH, the VL, or the linker of the scFv. In some embodiments, the cell binding domain is specific for a tag co-expressed by the engineered cell, such an EGFRt tag. In some embodiments, the cell binding domain is specific for a tag on a CAR construct that is expressed by the engineered cell. An example is shown in FIG. 5A. In some embodiments, such a tag on a CAR construct is part of the following structure: scFv-tag-transmembrane domain-additional domains such as a CD3zeta signaling domain, a co-stimulatory domain (e.g., a CD28 domain, a 41-BB domain)

In some embodiments, the cell binding domain comprises a domain from an antigen targeted by the engineered cell, non-limiting examples of which include, a neoepitope from a tumor-associated antigen, TSHR, CD19, CD123, CD22, CD30, CD171, CS-1, CLL-1, CD33, EGFRvlll, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11 Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51 E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1 B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1

Non-limiting examples of the cytokine includes IL-2, IL-7, IL-10, IL-15, and IL-21, or a functional fragment thereof, or a variant thereof, or any combinations thereof. In some embodiments, the cytokine is an IL-2 polypeptide, a fragment or a variant thereof. In some embodiments, the cytokine is an IL-7 polypeptide, a fragment or a variant thereof. In some embodiments, the cytokine is an IL-10 polypeptide, a fragment or a variant thereof. In some embodiments, the cytokine is an IL-21 polypeptide, a fragment or a variant thereof. Multiple modifications to an original cytokine peptide, e.g., wildtype IL-2, IL-7, IL-10, IL-15, or IL-21, may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. In some embodiments, a mutation or mutations are introduced to the polypeptide to modify its activity by reducing its affinity to its receptor.

### Interleukin-2 (IL-2)

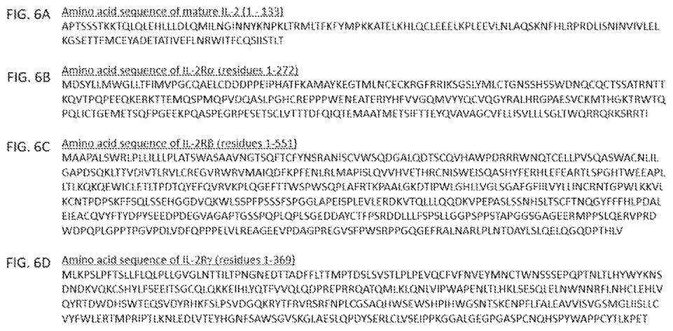

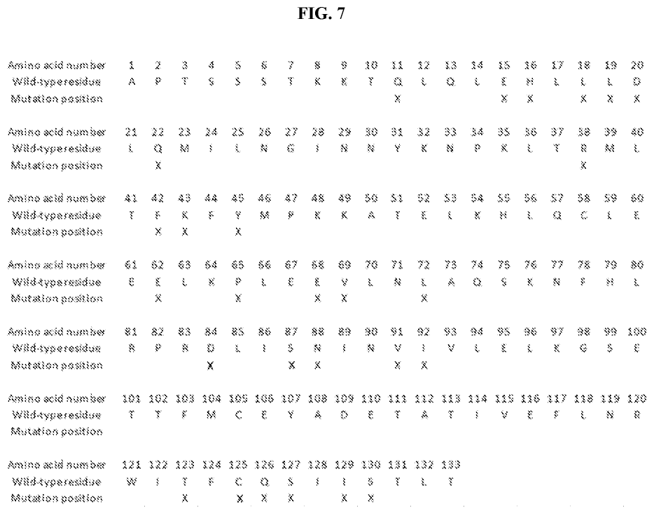

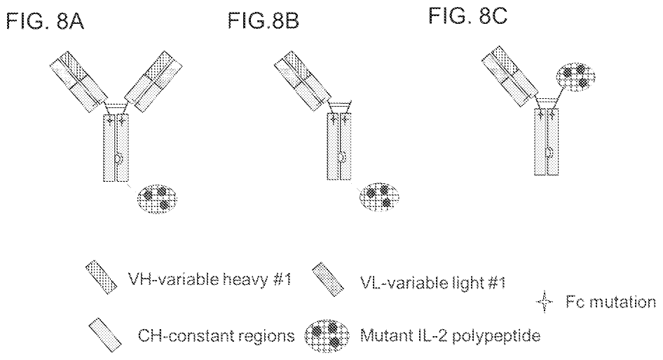

In some embodiments, the IL-2 polypeptide is a mutant IL-2 polypeptide as described herein. In some embodiments, provided herein are mutant cytokines (e.g., IL-2 polypeptides) that exhibit less than 50% of binding affinity to their receptors (e.g., IL-2Ra (e.g., comprising the amino acid sequence of SEQ ID NO: 2, or as depicted in FIG. 6B).

“Interleukin-2” or “IL-2” as used interchangeably herein can refer to any native IL-2, unless otherwise indicated. “IL-2” can encompass unprocessed IL-2 (such as precursor IL-2) as well as “mature IL-2” which can be a form of IL-2 that results from processing in the cell. A sequence of human “mature IL-2” is provided as SEQ ID NO: 1. One exemplary form of unprocessed human IL-2 may comprise of an additional N-terminal amino acid signal peptide attached to mature IL-2. “IL-2” can also include but is not limited to naturally occurring variants of IL-2, e.g., allelic or splice variants or variants. The amino acid sequence of an exemplary human IL-2 is described under UniProt P60568 (IL2 HUMAN). A “mutant IL-2 polypeptide” can refer to IL-2 polypeptide that can have altered affinity to its receptor, such as a reduced affinity to its receptor wherein such decreased affinity will result in reduced biological activity of the mutant. Alterations in affinity, such as reduction in affinity and thereby activity can be obtained by introducing a small number of amino acid mutations or substitutions. The mutant IL-2 polypeptides can also have other modifications to the peptide backbone, including but not limited to amino acid deletion, permutation, cyclization, disulfide bonds, or the post-translational modifications (e.g., glycosylation or altered carbohydrate) of a polypeptide, chemical or enzymatic modifications to the polypeptide (e.g., attaching PEG to the polypeptide backbone), addition of peptide tags or labels, or fusion to proteins or protein domains to generate a final construct with desired characteristics, such as reduced affinity to IL-2Rβγ. Desired activity may also include improved biophysical properties compared to the wild-type IL-2 polypeptide. Multiple modifications may be combined to achieve a desired activity modification, such as reduction or increase in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. In some cases, a mutation or mutations are introduced to the polypeptide to modify its activity.

In some embodiments, a wildtype IL-2 polypeptide comprises the sequence of:

(SEQ ID NO: 1)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFCQSIISTLT.

In some embodiments, mutant IL-2 polypeptides also exhibit less than 50% of binding affinity to IL-2RP (e.g., comprising the amino acid sequence of SEQ ID NO:3). In some embodiments, mutant IL-2 polypeptides exhibit less than 50% of binding affinity to IL-2Ra and less than 50% of binding affinity to IL-2RP (e.g., comprising the amino acid sequence of SEQ ID NO:3 or as depicted in FIG. 6C) compared to wild-type IL-2 polypeptide (e.g., comprising the amino acid sequence of SEQ ID NO:1). In some embodiments, mutant IL-2 polypeptides exhibit less than 50% of binding affinity to IL-2Rα and less than 50% of binding affinity to IL-2Rγ (e.g., comprising the amino acid sequence of SEQ ID NO:4 or as depicted in FIG. 6D), compared to wild-type IL-2 polypeptide (e.g., comprising the amino acid sequence of SEQ ID NO:1). In some embodiments, mutant IL-2 polypeptides exhibit less than 50% of binding affinity to IL-2Rα, less than 50% of binding affinity to IL-2Rβ, and less than 50% of binding affinity to IL-2Rγ, compared to wild-type IL-2 polypeptide. Differences in binding affinity of wild-type and disclosed mutant polypeptide for IL-2Rα and IL-2Rβ can be measured, e.g., in standard surface plasmon resonance (SPR) assays that measure affinity of protein-protein interactions familiar to those skilled in the art. Differences in binding affinity of wild-type and disclosed mutant polypeptide for IL-2Rγ cannot reliably be measured by SPR assays as the affinity of wild-type IL-2 polypeptide for IL-2Rγ is very low. Instead, their reduced affinity to IL-2Rγ can be deduced by performing an in vitro assay that measures pSTAT5 and compares the activity of IL-2 polypeptides with and without the IL-2Rγ affinity-reducing substitution on IL-2R-expressing cells.

In some embodiments, the cytokine is at least one of: (i) an IL-2Rβγ agonist polypeptide that binds to and/or activates an IL-2Rβ polypeptide comprising the amino acid sequence of SEQ ID NO: 3; and (ii) an IL-2Rβγ polypeptide agonist polypeptide that binds to and/or activates an IL-2Rγ polypeptide comprising the amino acid sequence of SEQ ID NO: 4.

In some embodiments the IL-2Rβ polypeptide comprises the sequence of:

(SEQ ID NO: 3)
MAAPALSWRLPLLILLLPLATSWASAAVNGTSQFTCFYNSRANISCVWS
QDGALQDTSCQVHAWPDRRRWNQTCELLPVSQASWACNLILGAPDSQKL
TTVDIVTLRVLCREGVRWRVMAIQDFKPFENLRLMAPISLQVVHVETHR
CNISWEISQASHYFERHLEFEARTLSPGHTWEEAPLLTLKQKQEWICLE
TLTPDTQYEFQVRVKPLQGEFTTWSPWSQPLAFRTKPAALGKDTIPWLG
HLLVGLSGAFGFIILVYLLINCRNTGPWLKKVLKCNTPDPSKFFSQLSS
EHGGDVQKWLSSPFPSSSFSPGGLAPEISPLEVLERDKVTQLLLQQDKV
PEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPPE
LVLREAGEEVPDAGPREGVSFPWSRPPGQGEFRALNARLPLNTDAYLSL
QELQGQDPTHLV.

In some embodiments the IL-2Rα polypeptide comprises the sequence of:

(SEQ ID NO: 2)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPGHCREPPPWENEATERIYHFVVGQM
VYYQCVQGYRALHRGPAESVCKMTHGKTRWTQPQLICTGEMETSQFPGE
EKPQASPEGRPESETSCLVTTTDFQIQTEMAATMETSIFTTEYQVAVAG
CVFLLISVLLLSGLTWQRRQRKSRRTI.

In some embodiments, the cytokine is an IL-2 polypeptide, or a functional fragment thereof, or a variant thereof. In some embodiments, the cytokine is a mutant IL-2 polypeptide that exhibits reduced binding affinity by 50% or more to an IL-2Rα polypeptide comprising the amino acid sequence of SEQ ID NO: 2, compared to binding affinity of a wild-type IL-2 polypeptide comprising the amino acid sequence of SEQ ID NO:1 to the IL-2Rα polypeptide. In some embodiments, the cytokine is a mutant IL-2 polypeptide that also exhibits reduced binding affinity by 50% or more to IL-2Rβ polypeptide comprising the amino acid sequence of SEQ ID NO: 3, compared to binding affinity of a wild-type IL-2 polypeptide comprising the amino acid sequence of SEQ ID NO: 1 to the IL-2Rβ polypeptide.

In some embodiments, the cytokine is a mutant IL-2 polypeptide that exhibits reduced binding affinity by 50% or more to an IL-2Rα polypeptide and reduced binding affinity by 50% or more to an IL-2Rγ polypeptide comprising the amino acid sequence of SEQ ID NO:4, compared to binding affinity of a wild-type IL-2 polypeptide comprising the amino acid sequence of SEQ ID NO: 1 to the IL-2Rγ polypeptide comprising the amino acid sequence of SEQ ID NO: 4.

In some embodiments, the IL2-Rγ polypeptide comprises the sequence of

(SEQ ID NO: 4)
:MLKPSLPFTSLLFLQLPLLGVGLNTTILTPNGNEDTTADFFLTTMPTD
SLSVSTLPLPEVQCFVFNVEYMNCTWNSSSEPQPTNLTLHYWYKNSDND
KVQKCSHYLFSEEITSGCQLQKKEIHLYQTFVVQLQDPREPRRQATQML
KLQNLVIPWAPENLTLHKLSESQLELNWNNRFLNHCLEHLVQYRTDWDH
SWTEQSVDYRHKFSLPSVDGQKRYTFRVRSRFNPLCGSAQHWSEWSHPI
HWGSNTSKENPFLFALEAVVISVGSMGLIISLLCVYFWLERTMPRIPTL
KNLEDLVTEYHGNFSAWSGVSKGLAESLQPDYSERLCLVSEIPPKGGAL
GEGPGASPCNQHSPYWAPPCYTLKPET.

The mutant IL-2 polypeptides of the present disclosure, in some examples, have one or more, two or more, or three or more affinity-reducing amino acid substitutions relative to the wild-type mature IL-2 polypeptide having an amino acid sequence of SEQ ID NO:1, wherein one or more, two or more, or three or more substituted residues, are selected from the following group: Q11, H16, L18, L19, D20, Q22, R38, F42, K43, Y45, E62, P65, E68, V69, L72, D84, S87, N88, V91, I92, T123, Q126, S127, I129, and S130. The location of possible amino acid substitutions in the sequence of the wild-type mature IL-2 polypeptide. Decreased affinity to IL-2Ra may be obtained by substituting one or more of the following residues in the sequence of the wild-type mature IL-2 polypeptide: R38, F42, K43, Y45, E62, P65, E68, V69, and L72. Decreased affinity to IL-2Rβ may be obtained by substituting one or more of the following residues: E15, H16, L19, D20, D84, S87, N88, V91, and 192. Decreased affinity to IL-2Rγ may be obtained by substituting one or more of the following residues in the sequence of the wild-type mature IL-2 polypeptide: Q11, L18, Q22, T123, Q126, S127, I129, and S130.

In some embodiments, the mutant IL-2 polypeptide comprises an F42A or F42K amino acid substitution relative to the wild-type mature IL-2 amino acid sequence, e.g., SEQ ID NO: 1, as depicted in FIG. 6A. In some embodiments, the mutant IL-2 polypeptide comprises an F42A or F42K amino acid substitution and an R38A, R38D, R38E, E62Q, E68A, E68Q, E68K, or E68R amino acid substitution relative to the wild-type mature IL-2 amino acid sequence. For example, in some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence, e.g., as shown in SEQ ID NO: 1. In some embodiments, the mutant IL-2 polypeptide comprises R38E and F42A amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to the wild-type IL-2 amino acid sequence. In some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence and an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to the wild-type mature IL-2 amino acid sequence, e.g., as shown in SEQ ID NO: 1. For example, in some embodiments, the mutant IL-2 polypeptide comprises R38E, F42A, and H16E amino acid substitutions relative to the wild-type IL-2 amino acid sequence. In some embodiments, the mutant IL-2 polypeptide comprises R38E, F42A, and H16D amino acid substitutions relative to the wild-type IL-2 amino acid sequence. In some embodiments, the mutant IL-2 polypeptide comprises R38E, F42A, and D84K amino acid substitutions relative to the wild-type IL-2 amino acid sequence. In some embodiments, the mutant IL-2 polypeptide comprises R38E, F42A, and D84R amino acid substitutions relative to the wild-type IL-2 amino acid sequence. In some embodiments, the mutant IL-2 polypeptide comprises R38E, F42A, and N88S amino acid substitutions relative to the wild-type IL-2 amino acid sequence. In some embodiments, the mutant IL-2 polypeptide comprises R38E, F42A, and N88A amino acid substitutions relative to the wild-type IL-2 amino acid sequence. In some embodiments, the mutant IL-2 polypeptide comprises R38E, F42A, and N88G amino acid substitutions relative to the wild-type IL-2 amino acid sequence. In some embodiments, the mutant IL-2 polypeptide comprises R38E, F42A, and N88R amino acid substitutions relative to the wild-type IL-2 amino acid sequence. In some embodiments, the mutant IL-2 polypeptide comprises R38E, F42A, and N88T amino acid substitutions relative to the wild-type IL-2 amino acid sequence. In some embodiments, the mutant IL-2 polypeptide comprises R38E, F42A, and N88D amino acid substitutions relative to the wild-type IL-2 amino acid sequence. In some embodiments, the mutant IL-2 polypeptide comprises R38E, F42A, and V91E amino acid substitutions relative to the wild-type IL-2 amino acid sequence. In some embodiments, the mutant IL-2 polypeptide comprises R38E, F42A, and Q126S amino acid substitutions relative to the wild-type IL-2 amino acid sequence. In some embodiments, the mutant IL-2 polypeptide comprises the amino acid sequence of SEQ ID NO: 1 with one of the following sets of amino acid substitutions (relative to the sequence of SEQ ID NO: 1): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88T, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, N88G, and C125A; R38A, F42K, N88R, and C125A; R38A, F42K, N88T, and C125A; R38A, F42K, N88D, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence of SEQ ID NO: 1 with one, two, three, four, or five amino acid substitutions relative to SEQ ID NO: 1, and wherein the one, two, three, four, or five substitution(s) comprise substitution(s) at positions of SEQ ID NO: 1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130. In some embodiments, the IL-2 polypeptide comprises the sequence of SEQ ID NO: 1 with one of the following sets of amino acid substitutions (relative to the sequence of SEQ ID NO: 1): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

In some embodiments, the IL-2 polypeptide comprises the sequence of SEQ ID NO: 1 with a further amino acid substitution relative to SEQ ID NO: 1 at position C125. In some embodiments, the IL-2 polypeptide comprises the sequence of SEQ ID NO: 1 with one of the following sets of amino acid substitutions (relative to the sequence of SEQ ID NO: 1): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

In some embodiments, the mutant IL-2 polypeptide comprises the amino acid sequence of

(SEQ ID NO: 11)
APTSSSTKKTQLQLEHLLLDLQMILNGINNYKNPKLTEMLTAKFYMPKK
ATELKHLQCLEEELKPLEEVLNLAQSKNFHLRPRDLISNINVIVLELKG
SETTFMCEYADETATIVEFLNRWITFCQSIISTLT.

In some embodiments, the mutant IL-2 polypeptide comprises the amino acid sequence of

(SEQ ID NO: 12)
APTSSSTKKTQLQLEHLLLDLQMILNGINNYKNPKLTDMLTAKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 13)
APTSSSTKKTQLQLEHLLLDLQMILNGINNYKNPKLTRMLTAKFYMPKKATELKHLQCL
EEQLKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 14)
APTSSSTKKTQLQLEHLLLDLQMILNGINNYKNPKLTAMLTKKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 15)
APTSSSTKKTQLQLEHLLLDLQMILNGINNYKNPKLTEMLTAKFYMPKKATELKHLQCL
EEELKPLEEVLNLAQSKNFHLRPRDLISSINVIVLELKGSETTFMCEYADETATIVEFLNR
WITFCQSIISTLT.

In some embodiments, the mutant IL-2 polypeptide comprises the amino acid sequence of

(SEQ ID NO: 16)
APTSSSTKKTQLQLEHLLLDLQMILNGINNYKNPKLTEMLTAKFYMPKKATELKHLQCL
EEELKPLEEVLNLAQSKNFHLRPRDLISAINVIVLELKGSETTFMCEYADETATIVEFLNR
WITFCQSIISTLT.

In some embodiments, the mutant IL-2 polypeptide comprises the amino acid sequence of

(SEQ ID NO: 17)
APTSSSTKKTQLQLEHLLLDLQMILNGINNYKNPKLTEMLTAKFYMPKKATELKHLQCL
EEELKPLEEVLNLAQSKNFHLRPRDLISNINEIVLELKGSETTFMCEYADETATIVEFLNR
WITFCQSIISTLT.

In some embodiments, the mutant IL-2 polypeptide comprises the amino acid sequence of

(SEQ ID NO: 18)
APTSSSTKKTQLQLEHLLLDLQMILNGINNYKNPKLTEMLTAKFYMPKKATELKHLQCL
EEELKPLEEVLNLAQSKNFHLRPRHLISNINVIVLELKGSETTFMCEYADETATIVEFLNR
WITFCQSIISTLT.

In some embodiments, the mutant IL-2 polypeptide comprises the amino acid sequence of

(SEQ ID NO: 19)
APTSSSTKKTQLQLEDLLLDLQMILNGINNYKNPKLTEMLTAKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 20)
APTSSSTKKTQLQLEELLLDLQMILNGINNYKNPKLTEMLTAKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 21)
APTSSSTKKTQLQLEHLLLDLQMILNGINNYKNPKLTEMLTAKFYMPKKATELKHLQCL
EEELKPLEEVLNLAQSKNFHLRPRDLISNINVIVLELKGSETTFMCEYADETATIVEFLNR
WITFCSSIISTLT.

In some embodiments, the mutant IL-2 polypeptide comprises the amino acid sequence of

(SEQ ID NO: 22)
APTSSSTKKTQLQLEHLLLDLQMILNGINNYKNPKLTDMLTAKFYMPKKATELKHLQCL
EEELKPLEEVLNLAQSKNFHLRPRDLISSINVIVLELKGSETTFMCEYADETATIVEFLNR
WITFCQSIISTLT.

In some embodiments, the mutant IL-2 polypeptide comprises the amino acid sequence of

(SEQ ID NO: 23)
APTSSSTKKTQLQLEHLLLDLQMILNGINNYKNPKLTDMLTAKFYMPKKATELKHLQCL
EEELKPLEEVLNLAQSKNFHLRPRDLISAINVIVLELKGSETTFMCEYADETATIVEFLNR
WITFCQSIISTLT.

In some embodiments, the mutant IL-2 polypeptide comprises the amino acid sequence of

(SEQ ID NO: 24)
APTSSSTKKTQLQLEHLLLDLQMILNGINNYKNPKLTDMLTAKFYMPKKATELKHLQCL.
EEELKPLEEVLNLAQSKNFHLRPRDLISNINEIVLELKGSETTFMCEYADETATIVEFLNR
WITFCQSIISTLT.

In some embodiments, the mutant IL-2 polypeptide comprises the amino acid sequence of

(SEQ ID NO: 25)
APTSSSTKKTQLQLEHLLLDLQMILNGINNYKNPKLTDMLTAKFYMPKKATELKHLQCL
EEELKPLEEVLNLAQSKNFHLRPRHLISNINVIVLELKGSETTFMCEYADETATIVEFLNR
WITFCQSIISTLT.

In some embodiments, the mutant IL-2 polypeptide comprises the amino acid sequence of

(SEQ ID NO: 26)
APTSSSTKKTQLQLEDLLLDLQMILNGINNYKNPKLTDMLTAKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 27)
APTSSSTKKTQLQLEELLLDLQMILNGINNYKNPKLTDMLTAKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 28)
APTSSSTKKTQLQLEHLLLDLQMILNGINNYKNPKLTDMLTAKFYMPKKATELKHLQCL
EEELKPLEEVLNLAQSKNFHLRPRDLISNINVIVLELKGSETTFMCEYADETATIVEFLNR
WITFCSSIISTLT.

In some embodiments, the mutant IL-2 polypeptide comprises the amino acid sequence of

(SEQ ID NO: 29)
APTSSSTKKTQLQLEHLLLDLQMILNGINNYKNPKLTAMLTKKFYMPKKATELKHLQCL
EEELKPLEEVLNLAQSKNFHLRPRDLISSINVIVLELKGSETTFMCEYADETATIVEFLNR
WITFCQSIISTLT.

In some embodiments, the mutant IL-2 polypeptide comprises the amino acid sequence of

(SEQ ID NO: 30)
APTSSSTKKTQLQLEHLLLDLQMILNGINNYKNPKLTAMLTKKFYMPKKATELKHLQCL
EEELKPLEEVLNLAQSKNFHLRPRDLISAINVIVLELKGSETTFMCEYADETATIVEFLNR
WITFCQSIISTLT.

In some embodiments, the mutant IL-2 polypeptide comprises the amino acid sequence of

(SEQ ID NO: 31)
APTSSSTKKTQLQLEHLLLDLQMILNGINNYKNPKLTAMLTKKFYMPKKATELKHLQCL
EEELKPLEEVLNLAQSKNFHLRPRDLISNINEIVLELKGSETTFMCEYADETATIVEFLNR
WITFCQSIISTLT.

In some embodiments, the mutant IL-2 polypeptide comprises the amino acid sequence of

(SEQ ID NO: 32)
APTSSSTKKTQLQLEHLLLDLQMILNGINNYKNPKLTAMLTKKFYMPKKATELKHLQCL
EEELKPLEEVLNLAQSKNFHLRPRHLISNINVIVLELKGSETTFMCEYADETATIVEFLNR
WITFCQSIISTLT.

In some embodiments, the mutant IL-2 polypeptide comprises the amino acid sequence of

(SEQ ID NO: 33)
APTSSSTKKTQLQLEDLLLDLQMILNGINNYKNPKLTAMLTKKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 34)
APTSSSTKKTQLQLEELLLDLQMILNGINNYKNPKLTAMLTKKFYMPKKATELKHLQCL
EEELKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 35)
APTSSSTKKTQLQLEHLLLDLQMILNGINNYKNPKLTAMLTKKFYMPKKATELKHLQCL
EEELKPLEEVLNLAQSKNFHLRPRDLISNINVIVLELKGSETTFMCEYADETATIVEFLNR
WITFCSSIISTLT.

In some embodiments, the mutant IL-2 polypeptide comprises the amino acid sequence of

(SEQ ID NO: 36)
APTSSSTKKTQLQLEHLLLDLQMILNGINNYKNPKLTRMLTAKFYMPKKATELKHLQCL
EEQLKPLEEVLNLAQSKNFHLRPRDLISSINVIVLELKGSETTFMCEYADETATIVEFLNR
WITFCQSIISTLT.

In some embodiments, the mutant IL-2 polypeptide comprises the amino acid sequence of

(SEQ ID NO: 37)
APTSSSTKKTQLQLEHLLLDLQMILNGINNYKNPKLTRMLTAKFYMPKKATELKHLQCL
EEQLKPLEEVLNLAQSKNFHLRPRDLISAINVIVLELKGSETTFMCEYADETATIVEFLNR
WITFCQSIISTLT.

In some embodiments, the mutant IL-2 polypeptide comprises the amino acid sequence of

(SEQ ID NO: 38)
APTSSSTKKTQLQLEHLLLDLQMILNGINNYKNPKLTRMLTAKFYMPKKATELKHLQCL
EEQLKPLEEVLNLAQSKNFHLRPRDLISNINEIVLELKGSETTFMCEYADETATIVEFLNR
WITFCQSIISTLT.

In some embodiments, the mutant IL-2 polypeptide comprises the amino acid sequence of

(SEQ ID NO: 39)
APTSSSTKKTQLQLEHLLLDLQMILNGINNYKNPKLTRMLTAKFYMPKKATELKHLQCL
EEQLKPLEEVLNLAQSKNFHLRPRHLISNINVIVLELKGSETTFMCEYADETATIVEFLNR
WITFCQSIISTLT.

In some embodiments, the mutant IL-2 polypeptide comprises the amino acid sequence of

(SEQ ID NO: 40)
APTSSSTKKTQLQLEDLLLDLQMILNGINNYKNPKLTRMLTAKFYMPKKATELKHLQCL
EEQLKPLEEVLNLAQSKNFHLRPRDLISNINVIVLELKGSETTFMCEYADETATIVEFLNR
WITFCQSIISTLT

In some embodiments, the mutant IL-2 polypeptide comprises the amino acid sequence of

(SEQ ID NO: 41)
APTSSSTKKTQLQLEELLLDLQMILNGINNYKNPKLTRMLTAKFYMPKKATELKHLQCL
EEQLKPLEEVLNLAQSKNFHLRPRDLISNINVIVLELKGSETTFMCEYADETATIVEFLNR
WITFCQSIISTLT.

In some embodiments, the mutant IL-2 polypeptide comprises the amino acid sequence of

(SEQ ID NO: 42)
APTSSSTKKTQLQLEHLLLDLQMILNGINNYKNPKLTRMLTAKFYMPKK
ATELKHLQCLEEQLKPLEEVLNLAQSKNFHLRPRDLISNINVIVLELKG
SETTFMCEYADETATIVEFLNRWITFCSSIISTLT.

In some embodiments, the mutant IL-2 polypeptide comprises the amino acid sequence of

(SEQ ID NO: 43)
APTSSSTKKTQLQLEHLLLDLQMILNGINNYKNPKLTEMLTAKFYMPKK
ATELKHLQCLEEELKPLEEVLNLAQSKNFHLRPRDLISNINVIVLELKG
SETTFMCEYADETATIVEFLNRWITFAQSIISTLT.

In some embodiments, the mutant IL-2 polypeptide comprises the amino acid sequence of

(SEQ ID NO: 44)
APTSSSTKKTQLQLEHLLLDLQMILNGINNYKNPKLTDMLTAKFYMPKK
ATELKHLQCLEEELKPLEEVLNLAQSKNFHLRPRDLISNINVIVLELKG
SETTFMCEYADETATIVEFLNRWITFAQSIISTLT.

In some embodiments, the mutant IL-2 polypeptide comprises the amino acid sequence of

(SEQ ID NO: 45)
APTSSSTKKTQLQLEHLLLDLQMILNGINNYKNPKLTRMLTAKFYMPKK
ATELKHLQCLEEQLKPLEEVLNLAQSKNFHLRPRDLISNINVIVLELKG
SETTFMCEYADETATIVEFLNRWITFAQSIISTLT.

In some embodiments, the mutant IL-2 polypeptide comprises the amino acid sequence of

(SEQ ID NO: 46)
APTSSSTKKTQLQLEHLLLDLQMILNGINNYKNPKLTAMLTKKFYMPKK
ATELKHLQCLEEELKPLEEVLNLAQSKNFHLRPRDLISNINVIVLELKG
SETTFMCEYADETATIVEFLNRWITFAQSIISTLT.

In some embodiments, the mutant IL-2 polypeptide comprises the amino acid sequence of

(SEQ ID NO: 47)
APTSSSTKKTQLQLEHLLLDLQMILNGINNYKNPKLTEMLTAKFYMPKK
ATELKHLQCLEEELKPLEEVLNLAQSKNFHLRPRDLISSINVIVLELKG
SETTFMCEYADETATIVEFLNRWITFAQSIISTLT.

In some embodiments, the mutant IL-2 polypeptide comprises the amino acid sequence of

(SEQ ID NO: 48)
APTSSSTKKTQLQLEHLLLDLQMILNGINNYKNPKLTEMLTAKFYMPKK
ATELKHLQCLEEELKPLEEVLNLAQSKNFHLRPRDLISAINVIVLELKG
SETTFMCEYADETATIVEFLNRWITFAQSIISTLT.

In some embodiments, the mutant IL-2 polypeptide comprises the amino acid sequence of

(SEQ ID NO: 49)
APTSSSTKKTQLQLEHLLLDLQMILNGINNYKNPKLTEMLTAKFYMPKK
ATELKHLQCLEEELKPLEEVLNLAQSKNFHLRPRDLISNINEIVLELKG
SETTFMCEYADETATIVEFLNRWITFAQSIISTLT.

In some embodiments, the mutant IL-2 polypeptide comprises the amino acid sequence of

(SEQ ID NO: 50)
APTSSSTKKTQLQLEHLLLDLQMILNGINNYKNPKLTEMLTAKFYMPKK
ATELKHLQCLEEELKPLEEVLNLAQSKNFHLRPRHLISNINVIVLELKG
SETTFMCEYADETATIVEFLNRWITFAQSIISTLT.

In some embodiments, the mutant IL-2 polypeptide comprises the amino acid sequence of

(SEQ ID NO: 51)
APTSSSTKKTQLQLEDLLLDLQMILNGINNYKNPKLTEMLTAKFYMPKK
ATELKHLQCLEEELKPLEEVLNLAQSKNFHLRPRDLISNINVIVLELKG
SETTFMCEYADETATIVEFLNRWITFAQSIISTLT.

In some embodiments, the mutant IL-2 polypeptide comprises the amino acid sequence of

(SEQ ID NO: 52)
APTSSSTKKTQLQLEELLLDLQMILNGINNYKNPKLTEMLTAKFYMPKK
ATELKHLQCLEEELKPLEEVLNLAQSKNFHLRPRDLISNINVIVLELKG
SETTFMCEYADETATIVEFLNRWITFAQSIISTLT.

In some embodiments, the mutant IL-2 polypeptide comprises the amino acid sequence of

(SEQ ID NO: 53)
APTSSSTKKTQLQLEHLLLDLQMILNGINNYKNPKLTEMLTAKFYMPKK
ATELKHLQCLEEELKPLEEVLNLAQSKNFHLRPRDLISNINVIVLELKG
SETTFMCEYADETATIVEFLNRWITFASSIISTLT.

In some embodiments, the mutant IL-2 polypeptide comprises the amino acid sequence of

(SEQ ID NO: 54)
APTSSSTKKTQLQLEHLLLDLQMILNGINNYKNPKLTDMLTAKFYMPKK
ATELKHLQCLEEELKPLEEVLNLAQSKNFHLRPRDLISSINVIVLELKG
SETTFMCEYADETATIVEFLNRWITFAQSIISTLT.

In some embodiments, the mutant IL-2 polypeptide comprises the amino acid sequence of

(SEQ ID NO: 55)
APTSSSTKKTQLQLEHLLLDLQMILNGINNYKNPKLTDMLTAKFYMPKK
ATELKHLQCLEEELKPLEEVLNLAQSKNFHLRPRDLISAINVIVLELKG
SETTFMCEYADETATIVEFLNRWITFAQSIISTLT.

In some embodiments, the mutant IL-2 polypeptide comprises the amino acid sequence of

(SEQ ID NO: 56)
APTSSSTKKTQLQLEHLLLDLQMILNGINNYKNPKLTDMLTAKFYMPKK
ATELKHLQCLEEELKPLEEVLNLAQSKNFHLRPRDLISNINEIVLELKG
SETTFMCEYADETATIVEFLNRWITFAQSIISTLT.

In some embodiments, the mutant IL-2 polypeptide comprises the amino acid sequence of

(SEQ ID NO: 57)
APTSSSTKKTQLQLEHLLLDLQMILNGINNYKNPKLTDMLTAKFYMPKKA
TELKHLQCLEEELKPLEEVLNLAQSKNFHLRPRHLISNINVIVLELKGSE
TTFMCEYADETATIVEFLNRWITFAQSIISTLT.

In some embodiments, the mutant IL-2 polypeptide comprises the amino acid sequence of

(SEQ ID NO: 58)
APTSSSTKKTQLQLEDLLLDLQMILNGINNYKNPKLTDMLTAKFYMPKKA
TELKHLQCLEEELKPLEEVLNLAQSKNFHLRPRDLISNINVIVLELKGSE
TTFMCEYADETATIVEFLNRWITFAQSIISTLT.

In some embodiments, the mutant IL-2 polypeptide comprises the amino acid sequence of

(SEQ ID NO: 59)
APTSSSTKKTQLQLEELLLDLQMILNGINNYKNPKLTDMLTAKFYMPKKA
TELKHLQCLEEELKPLEEVLNLAQSKNFHLRPRDLISNINVIVLELKGSE
TTFMCEYADETATIVEFLNRWITFAQSIISTLT.

In some embodiments, the mutant IL-2 polypeptide comprises the amino acid sequence of

(SEQ ID NO: 60)
APTSSSTKKTQLQLEHLLLDLQMILNGINNYKNPKLTDMLTAKFYMPKKA
TELKHLQCLEEELKPLEEVLNLAQSKNFHLRPRDLISNINVIVLELKGSE
TTFMCEYADETATIVEFLNRWITFASSIISTLT.

In some embodiments, the mutant IL-2 polypeptide comprises the amino acid sequence of

(SEQ ID NO: 61)
APTSSSTKKTQLQLEHLLLDLQMILNGINNYKNPKLTAMLTKKFYMPKKA
TELKHLQCLEEELKPLEEVLNLAQSKNFHLRPRDLISSINVIVLELKGSE
TTFMCEYADETATIVEFLNRWITFAQSIISTLT.

In some embodiments, the mutant IL-2 polypeptide comprises the amino acid sequence of

(SEQ ID NO: 62)
APTSSSTKKTQLQLEHLLLDLQMILNGINNYKNPKLTAMLTKKFYMPKKA
TELKHLQCLEEELKPLEEVLNLAQSKNFHLRPRDLISAINVIVLELKGSE
TTFMCEYADETATIVEFLNRWITFAQSIISTLT.

In some embodiments, the mutant IL-2 polypeptide comprises the amino acid sequence of

(SEQ ID NO: 63)
APTSSSTKKTQLQLEHLLLDLQMILNGINNYKNPKLTAMLTKKFYMPKKA
TELKHLQCLEEELKPLEEVLNLAQSKNFHLRPRDLISNINEIVLELKGSE
TTFMCEYADETATIVEFLNRWITFAQSIISTLT.

In some embodiments, the mutant IL-2 polypeptide comprises the amino acid sequence of

(SEQ ID NO: 64)
APTSSSTKKTQLQLEHLLLDLQMILNGINNYKNPKLTAMLTKKFYMPKKA
TELKHLQCLEEELKPLEEVLNLAQSKNFHLRPRHLISNINVIVLELKGSE
TTFMCEYADETATIVEFLNRWITFAQSIISTLT.

In some embodiments, the mutant IL-2 polypeptide comprises the amino acid sequence of

(SEQ ID NO: 65)
APTSSSTKKTQLQLEDLLLDLQMILNGINNYKNPKLTAMLTKKFYMPKKA
TELKHLQCLEEELKPLEEVLNLAQSKNFHLRPRDLISNINVIVLELKGSE
TTFMCEYADETATIVEFLNRWITFAQSIISTLT.

In some embodiments, the mutant IL-2 polypeptide comprises the amino acid sequence of

(SEQ ID NO: 66)
APTSSSTKKTQLQLEELLLDLQMILNGINNYKNPKLTAMLTKKFYMPKKA
TELKHLQCLEEELKPLEEVLNLAQSKNFHLRPRDLISNINVIVLELKGSE
TTFMCEYADETATIVEFLNRWITFAQSIISTLT.

In some embodiments, the mutant IL-2 polypeptide comprises the amino acid sequence of

(SEQ ID NO: 67)
APTSSSTKKTQLQLEHLLLDLQMILNGINNYKNPKLTAMLTKKFYMPKKA
TELKHLQCLEEELKPLEEVLNLAQSKNFHLRPRDLISNINVIVLELKGSE
TTFMCEYADETATIVEFLNRWITFASSIISTLT.

In some embodiments, the mutant IL-2 polypeptide comprises the amino acid sequence of

(SEQ ID NO: 68)
APTSSSTKKTQLQLEHLLLDLQMILNGINNYKNPKLTRMLTAKFYMPKKA
TELKHLQCLEEQLKPLEEVLNLAQSKNFHLRPRDLISSINVIVLELKGSE
TTFMCEYADETATIVEFLNRWITFAQSIISTLT.

In some embodiments, the mutant IL-2 polypeptide comprises the amino acid sequence of

(SEQ ID NO: 69)
APTSSSTKKTQLQLEHLLLDLQMILNGINNYKNPKLTRMLTAKFYMPKKA
TELKHLQCLEEQLKPLEEVLNLAQSKNFHLRPRDLISAINVIVLELKGSE
TTFMCEYADETATIVEFLNRWITFAQSIISTLT.

In some embodiments, the mutant IL-2 polypeptide comprises the amino acid sequence of

(SEQ ID NO: 70)
APTSSSTKKTQLQLEHLLLDLQMILNGINNYKNPKLTRMLTAKFYMPKKA
TELKHLQCLEEQLKPLEEVLNLAQSKNFHLRPRDLISNINEIVLELKGSE
TTFMCEYADETATIVEFLNRWITFAQSIISTLT.

In some embodiments, the mutant IL-2 polypeptide comprises the amino acid sequence of

(SEQ ID NO: 71)
APTSSSTKKTQLQLEHLLLDLQMILNGINNYKNPKLTRMLTAKFYMPKKA
TELKHLQCLEEQLKPLEEVLNLAQSKNFHLRPRHLISNINVIVLELKGSE
TTFMCEYADETATIVEFLNRWITFAQSIISTLT.

In some embodiments, the mutant IL-2 polypeptide comprises the amino acid sequence of

(SEQ ID NO: 72)
APTSSSTKKTQLQLEDLLLDLQMILNGINNYKNPKLTRML
TAKFYMPKKATELKHLQCLEEQLKPLEEVLNLAQSKNFHL
RPRDLISNINVIVLELKGSETTFMCEYADETATIVEFLNR
WITFAQSIISTLT.

In some embodiments, the mutant IL-2 polypeptide comprises the amino acid sequence of

(SEQ ID NO: 73)
APTSSSTKKTQLQLEELLLDLQMILNGINNYKNPKLTRML
TAKFYMPKKATELKHLQCLEEQLKPLEEVLNLAQSKNFHL
RPRDLISNINVIVLELKGSETTFMCEYADETATIVEFLNR
WITFAQSIISTLT.

In some embodiments, the mutant IL-2 polypeptide comprises the amino acid sequence of

(SEQ ID NO: 74)
APTSSSTKKTQLQLEHLLLDLQMILNGINNYKNPKLTRML
TAKFYMPKKATELKHLQCLEEQLKPLEEVLNLAQSKNFHL
RPRDLISNINVIVLELKGSETTFMCEYADETATIVEFLNR
WITFASSIISTLT.

In some embodiments, the mutant IL-2 polypeptide comprises the amino acid sequence of

(SEQ ID NO: 75)
APTSSSTKKTQLQLEHLLLDLQMILNGINNYKNPKLTRML
TAKFYMPKKATELKHLQCLEEELKPLEEVLNLAQSKNFHL
RPRDLISSINVIVLELKGSETTFMCEYADETATIVEFLNR
WITFAQSIISTLT.

In some embodiments, the mutant IL-2 polypeptide comprises the amino acid sequence of

(SEQ ID NO: 76)
APTSSSTKKTQLQLEHLLLDLQMILNGINNYKNPKLTRML
TAKFYMPKKATELKHLQCLEEELKPLEEVLNLAQSKNFHL
RPRDLISAINVIVLELKGSETTFMCEYADETATIVEFLNR
WITFAQSIISTLT.

In some embodiments, the mutant IL-2 polypeptide comprises the amino acid sequence of

(SEQ ID NO: 77)
APTSSSTKKTQLQLEHLLLDLQMILNGINNYKNPKLTRML
TAKFYMPKKATELKHLQCLEEELKPLEEVLNLAQSKNFHL
RPRDLISNINEIVLELKGSETTFMCEYADETATIVEFLNR
WITFAQSIISTLT.

In some embodiments, the mutant IL-2 polypeptide comprises the amino acid sequence of

(SEQ ID NO: 78)
APTSSSTKKTQLQLEHLLLDLQMILNGINNYKNPKLTRML
TAKFYMPKKATELKHLQCLEEELKPLEEVLNLAQSKNFHL
RPRHLISNINVIVLELKGSETTFMCEYADETATIVEFLNR
WITFAQSIISTLT.

In some embodiments, the mutant IL-2 polypeptide comprises the amino acid sequence of

(SEQ ID NO: 79)
APTSSSTKKTQLQLEDLLLDLQMILNGINNYKNPKLTRML
TAKFYMPKKATELKHLQCLEEELKPLEEVLNLAQSKNFHL
RPRDLISNINVIVLELKGSETTFMCEYADETATIVEFLNR
WITFAQSIISTLT.

In some embodiments, the mutant IL-2 polypeptide comprises the amino acid sequence of

(SEQ ID NO: 80)
APTSSSTKKTQLQLEELLLDLQMILNGINNYKNPKLTRML
TAKFYMPKKATELKHLQCLEEELKPLEEVLNLAQSKNFHL
RPRDLISNINVIVLELKGSETTFMCEYADETATIVEFLNR
WITFAQSIISTLT.

In some embodiments, the mutant IL-2 polypeptide comprises the amino acid sequence of

(SEQ ID NO: 81)
APTSSSTKKTQLQLEHLLLDLQMILNGINNYKNPKLTRML
TAKFYMPKKATELKHLQCLEEELKPLEEVLNLAQSKNFHL
RPRDLISNINVIVLELKGSETTFMCEYADETATIVEFLNR
WITFASSIISTLT.

In some embodiments, the mutant IL-2 polypeptide comprises the amino acid sequence of

(SEQ ID NO: 82)
APTSSSTKKTQLQLEHLLLDLQMILNGINNYKNPKLTEML
TAKFYMPKKATELKHLQCLEEELKPLEEVLNLAQSKNFHL
RPRDLISGINVIVLELKGSETTFMCEYADETATIVEFLNR
WITFCQSIISTLT.

In some embodiments, the mutant IL-2 polypeptide comprises the amino acid sequence of

(SEQ ID NO: 83)
APTSSSTKKTQLQLEHLLLDLQMILNGINNYKNPKLTDML
TAKFYMPKKATELKHLQCLEEELKPLEEVLNLAQSKNFHL
RPRDLISGINVIVLELKGSETTFMCEYADETATIVEFLNR
WITFCQSIISTLT.

In some embodiments, the mutant IL-2 polypeptide comprises the amino acid sequence of

(SEQ ID NO: 84)
APTSSSTKKTQLQLEHLLLDLQMILNGINNYKNPKLTAML
TKKFYMPKKATELKHLQCLEEELKPLEEVLNLAQSKNFHL
RPRDLISGINVIVLELKGSETTFMCEYADETATIVEFLNR
WITFCQSIISTLT.

In some embodiments, the mutant IL-2 polypeptide comprises the amino acid sequence of

(SEQ ID NO: 85)
APTSSSTKKTQLQLEHLLLDLQMILNGINNYKNPKLTRML
TAKFYMPKKATELKHLQCLEEQLKPLEEVLNLAQSKNFHL
RPRDLISGINVIVLELKGSETTFMCEYADETATIVEFLNR
WITFCQSIISTLT

In some embodiments, the mutant IL-2 polypeptide comprises the amino acid sequence of

(SEQ ID NO: 86)
APTSSSTKKTQLQLEHLLLDLQMILNGINNYKNPKLTEML
TAKFYMPKKATELKHLQCLEEELKPLEEVLNLAQSKNFHL
RPRDLISGINVIVLELKGSETTFMCEYADETATIVEFLNR
WITFAQSIISTLT.

In some embodiments, the mutant IL-2 polypeptide comprises the amino acid sequence of

(SEQ ID NO: 87)
APTSSSTKKTQLQLEHLLLDLQMILNGINNYKNPKLTDMLTAKFYMPKK
ATELKHLQCLEEELKPLEEVLNLAQSKNFHLRPRDLISGINVIVLELKG
SETTFMCEYADETATIVEFLNRWITFAQSIISTLT.

In some embodiments, the mutant IL-2 polypeptide comprises the amino acid sequence of

(SEQ ID NO: 88)
APTSSSTKKTQLQLEHLLLDLQMILNGINNYKNPKLTAMLTKKFYMPKK
ATELKHLQCLEEELKPLEEVLNLAQSKNFHLRPRDLISGINVIVLELKG
SETTFMCEYADETATIVEFLNRWITFAQSIISTLT.

In some embodiments, the mutant IL-2 polypeptide comprises the amino acid sequence of

(SEQ ID NO: 89)
APTSSSTKKTQLQLEHLLLDLQMILNGINNYKNPKLTRMLTAKFYMPKK
ATELKHLQCLEEQLKPLEEVLNLAQSKNFHLRPRDLISGINVIVLELKG
SETTFMCEYADETATIVEFLNRWITFAQSIISTLT.

In some embodiments, the mutant IL-2 polypeptide comprises the amino acid sequence of

(SEQ ID NO: 90)
APTSSSTKKTQLQLEHLLLDLQMILNGINNYKNPKLTRMLTAKFYMPKK
ATELKHLQCLEEELKPLEEVLNLAQSKNFHLRPRDLISGINVIVLELKG
SETTFMCEYADETATIVEFLNRWITFAQSIISTLT.

In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRKLISNINVIVLELKGSETTFMCEYADETATIVEFLNR WITFCQSIISTLT (SEQ ID NO:137). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRRLISNINVIVLELKGSETTFMCEYADETATIVEFLNR WITFCQSIISTLT (SEQ ID NO:138). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRKLISNINVIVLELKGSETTFMCEYADETATIVEFLNR WITFCQSIISTLT (SEQ ID NO:139). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRRLISNINVIVLELKGSETTFMCEYADETATIVEFLNR WITFCQSIISTLT (SEQ ID NO:140). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRKLISNINVIVLELKGSETTFMCEYADETATIVEFLNR WITFCQSIISTLT (SEQ ID NO:141). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRRLISNINVIVLELKGSETTFMCEYADETATIVEFLNR WITFCQSIISTLT (SEQ ID NO:142). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRKLISNINVIVLELKGSETTFMCEYADETATIVEFLNR WITFCQSIISTLT (SEQ ID NO:143). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRRLISNINVIVLELKGSETTFMCEYADETATIVEFLNR WITFCQSIISTLT (SEQ ID NO:144). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRKLISNINVIVLELKGSETTFMCEYADETATIVEFLNR WITFAQSIISTLT (SEQ ID NO:145). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRRLISNINVIVLELKGSETTFMCEYADETATIVEFLNR WITFAQSIISTLT (SEQ ID NO:146). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRKLISNINVIVLELKGSETTFMCEYADETATIVEFLNR WITFAQSIISTLT (SEQ ID NO:147). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRRLISNINVIVLELKGSETTFMCEYADETATIVEFLNR WITFAQSIISTLT (SEQ ID NO:148). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRKLISNINVIVLELKGSETTFMCEYADETATIVEFLNR WITFAQSIISTLT (SEQ ID NO:149). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRRLISNINVIVLELKGSETTFMCEYADETATIVEFLNR WITFAQSIISTLT (SEQ ID NO:150). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRKLISNINVIVLELKGSETTFMCEYADETATIVEFLNR WITFAQSIISTLT (SEQ ID NO:151). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRRLISNINVIVLELKGSETTFMCEYADETATIVEFLNR WITFAQSIISTLT (SEQ ID NO:152). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRKLISNINVIVLELKGSETTFMCEYADETATIVEFLNR WITFAQSIISTLT (SEQ ID NO:153). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRRLISNINVIVLELKGSETTFMCEYADETATIVEFLNR WITFAQSIISTLT (SEQ ID NO:154). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO:156). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:157). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:158). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISRINVIVLELKGSETTFMCEYADETATIVEFLNR WITFCQSIISTLT (SEQ ID NO:159). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISTINVIVLELKGSETTFMCEYADETATIVEFLNR WITFCQSIISTLT (SEQ ID NO:160). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISDINVIVLELKGSETTFMCEYADETATIVEFLNR WITFCQSIISTLT (SEQ ID NO:161). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISRINVIVLELKGSETTFMCEYADETATIVEFLNR WITFCQSIISTLT (SEQ ID NO:162). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISTINVIVLELKGSETTFMCEYADETATIVEFLNR WITFCQSIISTLT (SEQ ID NO:163). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISDINVIVLELKGSETTFMCEYADETATIVEFLNR WITFCQSIISTLT (SEQ ID NO:164). In some embodiments, the mutant IL-2 polypeptide comprises the amino acid sequence of APT SSSTKKTQLQLEHLLLDLQMILNGINNYKNPKLTAMLTKKFYMPKKATELKHLQCL EEELKPLEEVLNLAQSKNFHLRPRDLISRINVIVLELKGSETTFMCEYADETATIVEFLNR WITFCQSIISTLT (SEQ ID NO:165). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISTINVIVLELKGSETTFMCEYADETATIVEFLNR WITFCQSIISTLT (SEQ ID NO:166). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISDINVIVLELKGSETTFMCEYADETATIVEFLNR WITFCQSIISTLT (SEQ ID NO:167). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISRINVIVLELKGSETTFMCEYADETATIVEFLNR WITFCQSIISTLT (SEQ ID NO:168). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISTINVIVLELKGSETTFMCEYADETATIVEFLNR WITFCQSIISTLT (SEQ ID NO:169). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISDINVIVLELKGSETTFMCEYADETATIVEFLNR WITFCQSIISTLT (SEQ ID NO:170). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:174). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:175). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO:176). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISRINVIVLELKGSETTFMCEYADETATIVEFLNR WITFAQSIISTLT (SEQ ID NO:177). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISTINVIVLELKGSETTFMCEYADETATIVEFLNR WITFAQSIISTLT (SEQ ID NO:178). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISDINVIVLELKGSETTFMCEYADETATIVEFLNR WITFAQSIISTLT (SEQ ID NO:179). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISRINVIVLELKGSETTFMCEYADETATIVEFLNR WITFAQSIISTLT (SEQ ID NO:180). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISTINVIVLELKGSETTFMCEYADETATIVEFLNR WITFAQSIISTLT (SEQ ID NO:181). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISDINVIVLELKGSETTFMCEYADETATIVEFLNR WITFAQSIISTLT (SEQ ID NO:182). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISRINVIVLELKGSETTFMCEYADETATIVEFLNR WITFAQSIISTLT (SEQ ID NO:183). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISTINVIVLELKGSETTFMCEYADETATIVEFLNR WITFAQSIISTLT (SEQ ID NO:184). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISDINVIVLELKGSETTFMCEYADETATIVEFLNR WITFAQSIISTLT (SEQ ID NO: 185). In some embodiments, the mutant T1L-2 polypeptide comprises the amino acid sequence of an IL-2 polypeptide listed in Table 7.

TABLE 7
Exemplary IL-2 polypeptide sequences

| IL-2 | | SEQ ID |
|---|---|---|
| ID | Sequence | NO |
| m1 | APTSSSTKKTQLQLEHLLLDLQMILNGINNYKNPKLTEMLTAKFYMPKKATELKHL | 43 |
| | QCLEEELKPLEEVLNLAQSKNFHLRPRDLISNINVIVLELKGSETTEMCEYADETA | |
| | TIVEFLNRWITFAQSIISTLT | |
| m2 | APTSSSTKKTQLQLEHLLLDLQMILNGINNYKNPKLTEMLTAKFYMPKKATELKHL | 48 |
| | QCLEEELKPLEEVLNLAQSKNFHLRPRDLISAINVIVLELKGSETTEMCEYADETA | |
| | TIVEFLNRWITFAQSIISTLT | |
| m3 | APTSSSTKKTQLQLEELLLDLQMILNGINNYKNPKLTEMLTAKFYMPKKATELKHL | 52 |
| | QCLEEELKPLEEVLNLAQSKNFHLRPRDLISNINVIVLELKGSETTEMCEYADETA | |
| | TIVEFLNRWITFAQSIISTLT | |
| m4 | APTSSSTKKTQLQLEHLLLDLQMILNGINNYKNPKLTEMLTAKFYMPKKATELKHL | 49 |
| | QCLEEELKPLEEVLNLAQSKNFHLRPRDLISNINEIVLELKGSETTEMCEYADETA | |
| | TIVEFLNRWITFAQSIISTLT | |
| m5 | APTSSSTKKTQLQLEHLLLDLQMILNGINNYKNPKLTEMLTAKFYMPKKATELKHL | 156 |
| | QCLEEELKPLEEVLNLAQSKNFHLRPRDLISDINVIVLELKGSETTEMCEYADETA | |
| | TIVEFLNRWITFAQSIISTLT | |

In some embodiments, the mutant IL-2 polypeptides of the present disclosure also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell. For example, residue C125 may be replaced with a neutral amino acid such as serine, alanine, threonine or valine; and N terminal A1 residue could be deleted, both of which were described in U.S. Pat. No. 4,518,584. Mutant T1L-2 polypeptides may also include a mutation of the residue M104, such as M104A, as described in U.S. Pat. No. 5,206,344. Thus, in certain embodiments the mutant IL-2 polypeptide of the present disclosure comprises the amino acid substitution C125A. In other embodiments, one, two, or three N-terminal residues are deleted.

### Interleukin-10 (IL-10)

Interleukin-10 (IL-10) is a cytokine that regulates many immune cell subsets, some of which include monocytes, macrophages, dendritic cells, B cells, T cells, NTK cells, and others. IL-10 binds to a heterodimeric receptor (IL-10 receptor, IL-10R) that consists of two subunits, IL-10RA, specific to IL-10 and expressed mostly on immune cells, and IL-10RB, shared with other cytokines and expressed more broadly. Binding of IL-10 to its receptor induces the phosphorylation of receptor-associated Janus kinase, JAK1, and Tyrosine kinase, TYK2, which promotes the phosphorylation of STAT3 transcription factor (pSTAT3) that regulates the transcription of many genes in lymphocytes.

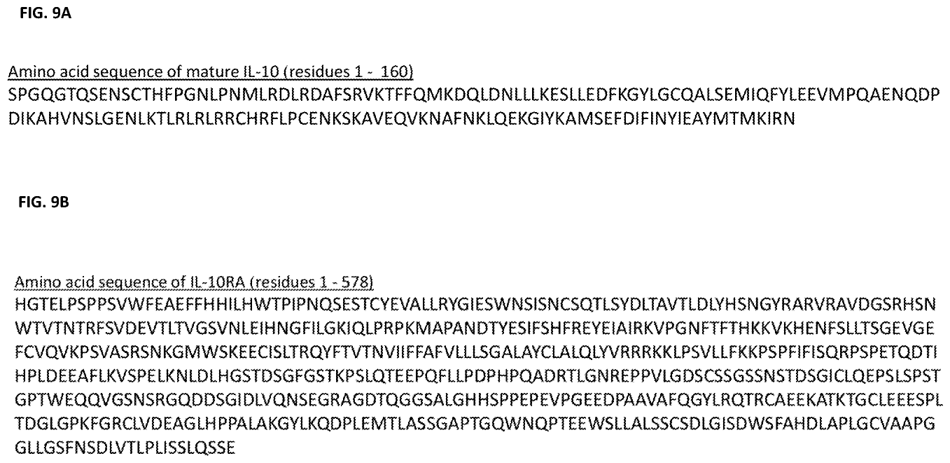

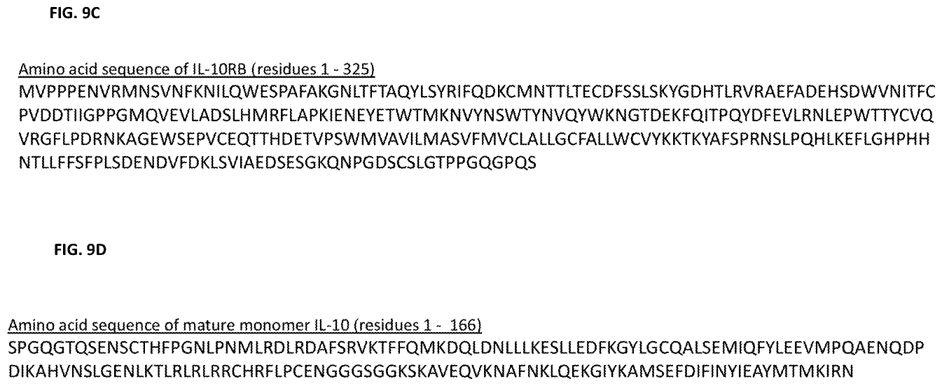

The term “Interleukin-10” or “IL-10,” as used herein interchangeably, can refer to any native IL-10 from any vertebrate source, including mammals such as primates (e.g. humans) and rodents (e.g., mice and rats), unless otherwise indicated. IL-10 can exist as a homodimer. “IL-10” can encompass unprocessed IL-10 as well as “mature IL-10” which is a form of IL-10 that results from processing in the cell. The sequence of “mature IL-10” is depicted in FIG. 9A. One exemplary form of unprocessed human IL-10 comprises of an additional N-terminal amino acid signal peptide attached to mature IL-10. “IL-10” can also include naturally occurring variants of IL-10, e.g., allelic or splice variants or variants. The amino acid sequence of an exemplary human IL-10 is described under UniProt P22301 (IL10_HUMAN).

“IL-10 homodimer” or “IL-10 dimer,” as used herein interchangeably, can refer to a naturally symmetric homodimer form of wild-type IL-10 polypeptide that binds to a tetrameric IL-10 receptor (IL-10R) complex on the cell, consisting of 2 molecules of IL-10R α-chain (IL-10RA) and two molecules of the IL-10R j-chain (IL-10RB). The α-helices from each IL-10 polypeptide chain intertwine such that the first four helices of one chain (A-D) associate with the last two helices (E and F) of the other, hereby maintaining structural integrity of each domain when dimerized (Walter & Nagabhushan, Biochemistry. 1995 Sep. 26; 34(38):12118-25). “IL-10 monomer” can refer to a monomeric form of IL-10 that can be generated by extending the loop that connects the swapped secondary structural elements. As described in Josephson et al, Biochemistry 1995 Sep. 26; 34(38):12118-25, insertion of 6 amino acids into the said loop was sufficient to prevent dimerization and induce IL-10 monomer formation. The resulting IL-10 monomer was biologically active and capable of binding to a single IL-10RA molecule and recruiting a single IL-10RB molecule into the signaling complex to elicit IL-10-mediated cellular responses. Therefore, inserting a short amino acid sequence or a short linker into the sequence of an IL-10 polypeptide (e.g., wild-type IL-10 or any mutant IL-10 polypeptide of the present disclosure) between loop D (ends with residue C114) and loop E (begins with residue V121) generates a “monomeric isomer” of said IL-10 polypeptide. This added amino acid sequence or linker can be inserted immediately after C114, E115, N116, K117, S118, K119, or A120. As described herein, the amino acid numbering for an IL-10 monomer polypeptide is based on the number of SEQ ID NO: 95 (i.e., an IL-10 dimer polypeptide), such that the linker sequence/amino acid(s) are not counted.

“Mutant IL-10 polypeptide” can refer to an IL-10 polypeptide that has an amino acid sequence different from a wild type IL-10. For example, a mutant IL-10 polypeptide may have amino acid substitutions, deletions, and insertions. In some embodiments, a mutant IL-10 polypeptide has reduced affinity to its receptor wherein such decreased affinity results in reduced biological activity of the mutant. Reduction in affinity and thereby activity can be obtained by introducing a small number of amino acid mutations or substitutions. The mutant IL-10 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-10R. Desired activity may also include improved biophysical properties compared to the wild-type IL-10 polypeptide.

In some embodiments, the present disclosure relates to mutant IL-10 polypeptides, and targeted cytokine comprising a mutant IL-10 polypeptide. In some embodiments, the mutant IL-10 polypeptides comprise one or more mutations (e.g., relative to SEQ ID NO: 95) that increase binding affinity to an IL-10RB polypeptide (e.g., comprising the sequence of SEQ ID NO: 97). In some embodiments, the mutant IL-10 polypeptides comprise one or more mutations (e.g., relative to SEQ ID NO: 95) that decrease binding affinity to an IL-10RA polypeptide (e.g., comprising the sequence of SEQ ID NO: 96). In some embodiments, the mutant IL-10 polypeptides comprise one or more mutations (e.g., relative to SEQ ID NO: 95) that increase binding affinity to an IL-10RB polypeptide (e.g., comprising the sequence of SEQ ID NO: 97) and comprise one or more mutations (e.g., relative to SEQ ID NO: 95) that decrease binding affinity to an IL-10RA polypeptide (e.g., comprising the sequence of SEQ ID NO: 96). In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of either wild-type mature IL-10 (e.g., SEQ ID NO: 95 depicted in FIG. 9A), or the mature monomer IL-10 depicted (e.g., SEQ ID NO: 98 depicted in FIG. 9D).

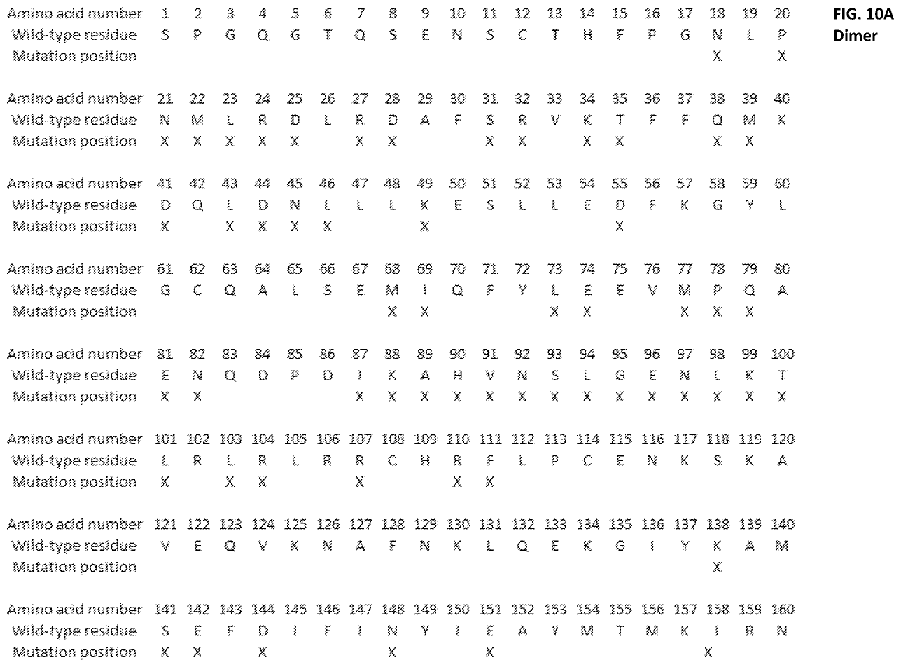

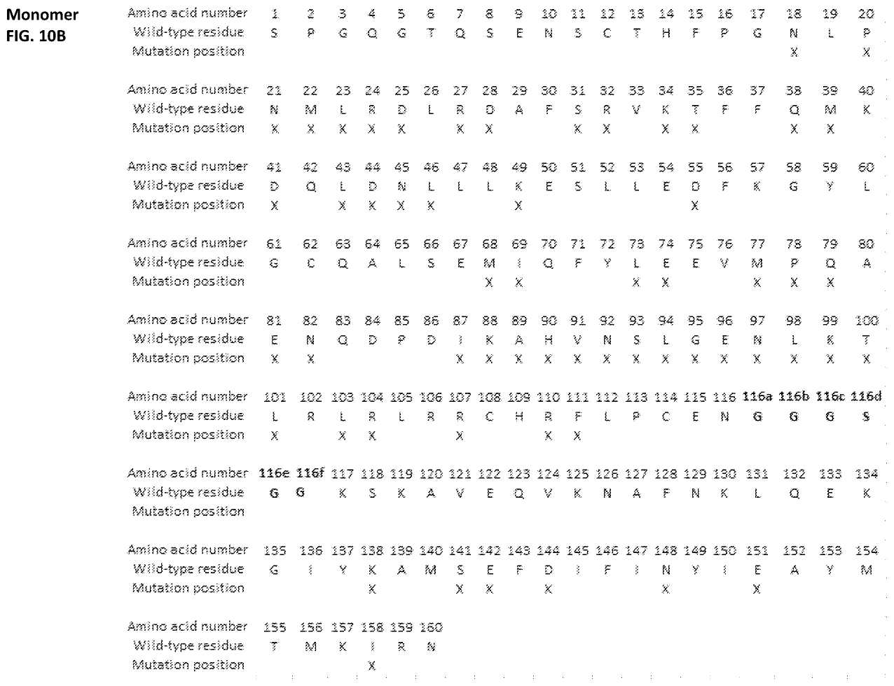

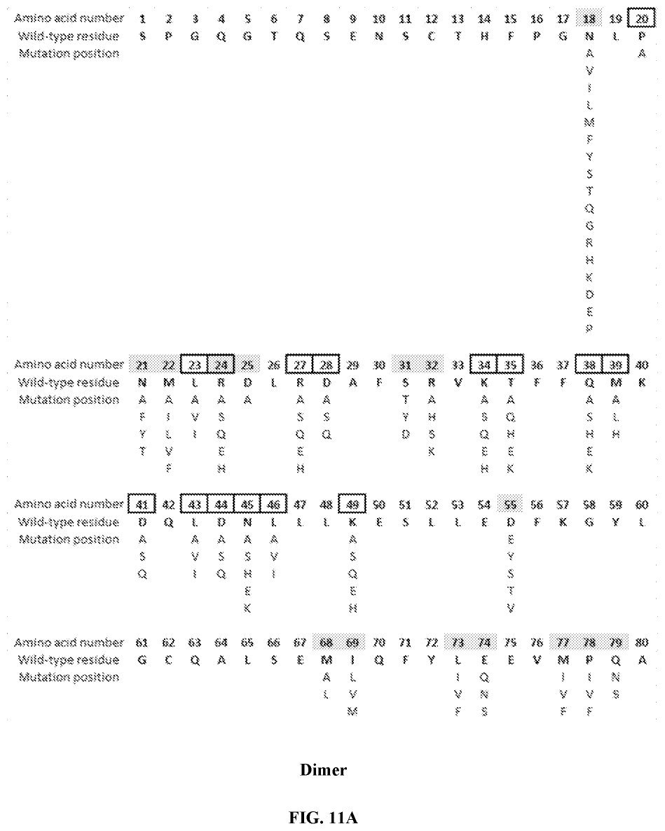

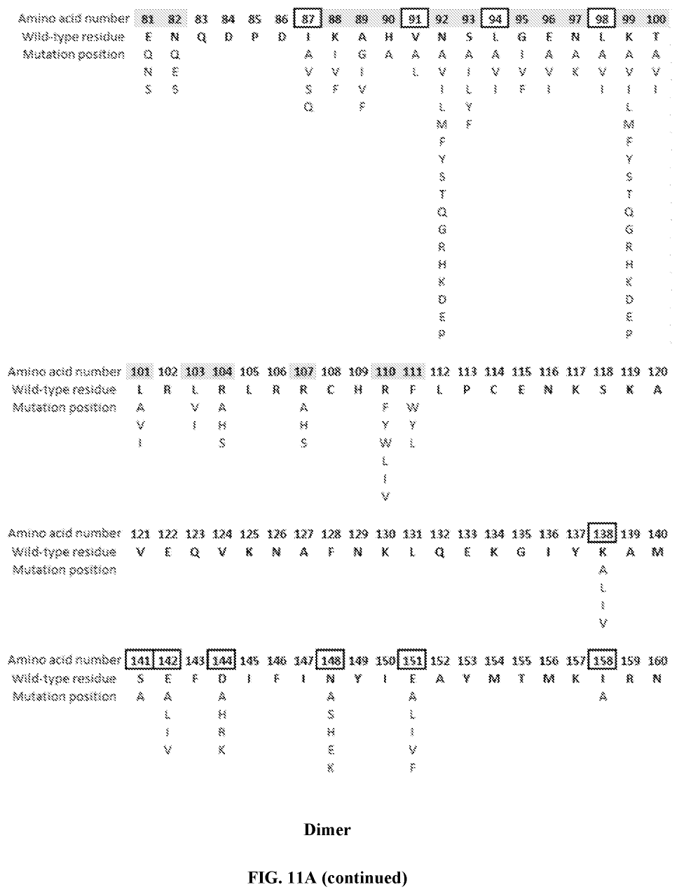

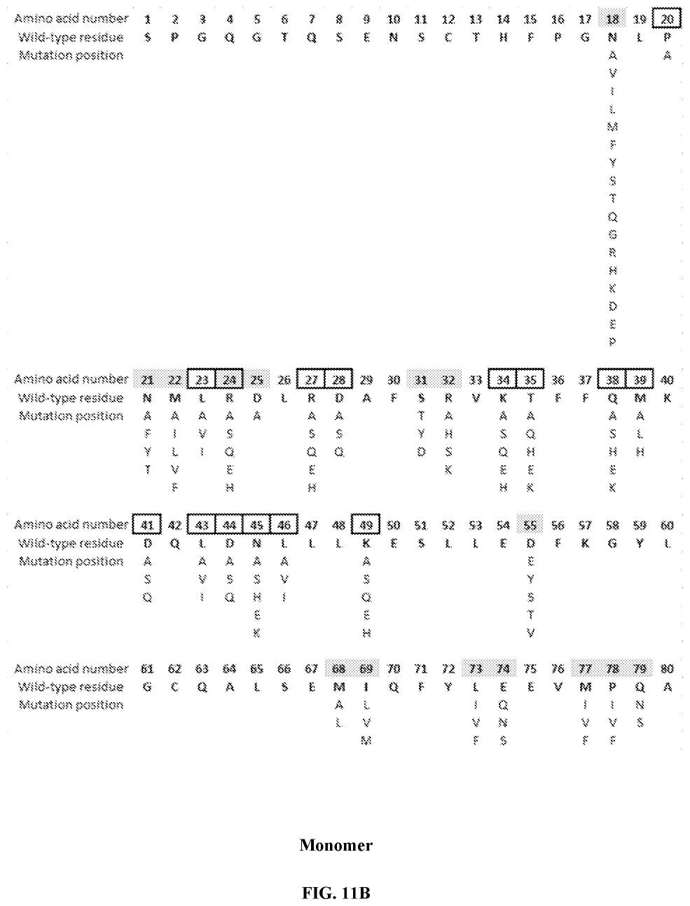

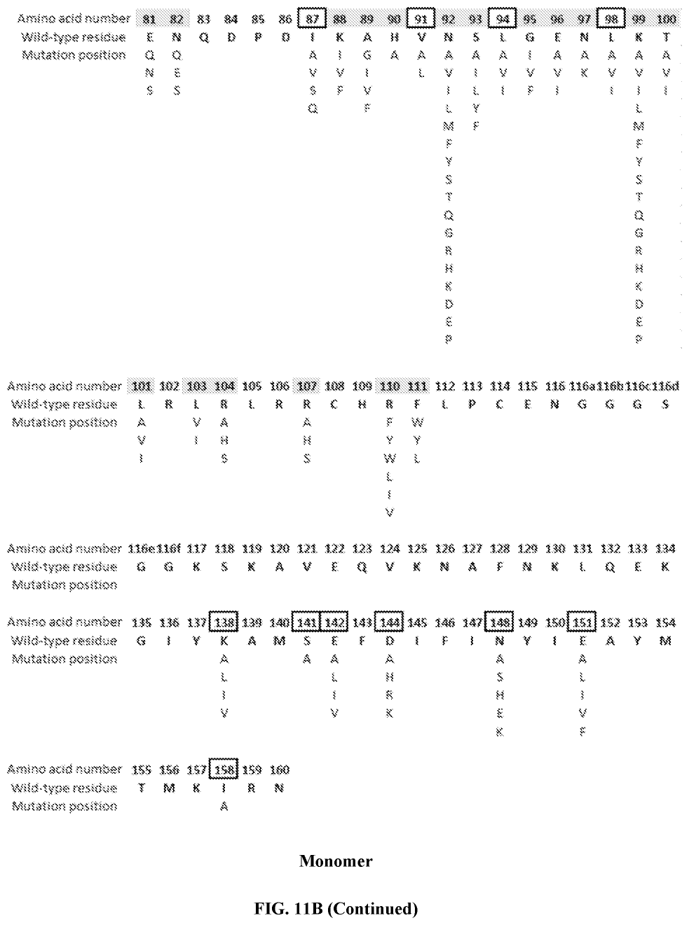

In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of either wild-type mature IL-10 (e.g., SEQ ID NO: 95 depicted in FIG. 9A), or the mature monomer IL-10 (e.g., SEQ ID NO: 98 depicted in FIG. 9D). In some embodiments, the mutant IL-10 polypeptide: i) exhibits reduced binding affinity to IL-10RA polypeptide having an amino acid sequence as set forth by SEQ ID NO: 96 depicted in FIG. 9B; and ii) has one or more amino acid substitutions relative to the amino acid sequence of the wild-type IL-10 polypeptide as set forth by SEQ ID NO: 95 depicted in FIG. 9A or the mature monomer IL-10 as set forth by SEQ ID NO: 98 depicted in FIG. 9D and selected from a group of: P20, L23, R24, R27, D28, K34, T35, Q38, M39, D41, L43, D44, N45, L46, K49, 187, V91, L94, L98, K138, S141, E142, D144, N148, E151, and 1158, as depicted in FIGS. 10A-11B. In some embodiments, the one or more amino acid substitutions are selected from the group consisting of: R24A, R27A, K34A, K34D, K34E, K34S, K34P, K34G, K34T, K34H, K34L, K34N, K34F, K34R, K34Q, K34V, K34Y, Q38A, Q38D, Q38P, Q38G, Q38H, Q38I, Q38L, Q38R, Q38K, Q38N, Q38F, Q38T, Q38E, Q38S, Q38V, Q38Y, D44A, D44E, D44S, D44V, D44G, D44H, D44I, D44K, D44P, D44L, D44N, D44F, D44T, D44R, D44Q, I87A, K138A, E142A, E142G, E142N, E142L, E142F, E142I, E142V, E142K, E142R, E142P, E142Q, E142T, E142S, E142Y, D144A, D144E, D144G, D144H, D144R, D1441, D144K, D144N, D144Q, D144P, D144S, D144L, D144T, D144V, D144Y, N148G, N148P, N148S, N148D, N148T, N148K, N148V, N148I, N148E, N148F, E151A, E151G, E151H, E151I, E151N, E151F, E151L, E151V, E151R, E151K, E151P, E151Q, E151S, E151T, and E151Y. In some embodiments, the one or more amino acid substitutions are selected from the group consisting of: R24A, R27A, K34A, K34D, K34E, K34S, K34P, K34G, K34T, K34H, K34L, K34N, K34F, K34V, K34Y, Q38A, Q38D, Q38P, Q38G, Q38I, Q38L, Q38R, Q38K, Q38F, Q38T, Q38E, Q38S, Q38V, Q38Y, I87A, K138A, E142A, E142G, E142N, E142L, E142F, E142I, E142V, E142K, E142R, E142P, E142Q, E142T, E142S, E142Y, D144A, D144E, D144G, D144H, D144R, D1441, D144K, D144N, D144Q, D144P, D144S, D144L, D144T, D144V, D144Y, N148P, N148D, N148I, E151A, E151G, E151H, E151I, E151N, E151F, E151L, E151V, E151R, E151K, E151P, E151Q, E151S, E151T, and E151Y. In some embodiments, the mutant IL-10 polypeptide of the present disclosure exhibits reduced binding affinity by 50% or more to IL-10RA polypeptide having an amino acid sequence as set forth by SEQ ID NO: 96 depicted in FIG. 9B. Differences in binding affinity of the wild-type and mutant IL-10 polypeptides to IL-10RA are measured in standard SPR assays that measure affinity of protein-protein interactions familiar to those skilled in the art. In some embodiments, the mutant IL-10 polypeptide comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the amino acid sequence of either wild-type mature IL-10 (e.g., SEQ ID NO: 95 depicted in FIG. 9A), or the mature monomer IL-10 (e.g., SEQ ID NO: 98 depicted in FIG. 9D). In some embodiments, the mutant IL-10 polypeptide: i) exhibits increased binding affinity to IL-10RB polypeptide having an amino acid sequence as set forth by SEQ ID NO: 97 depicted in FIG. 9C; and ii) has one or more amino acid substitutions relative to the amino acid sequence of the wild-type mature IL-10 polypeptide as set forth by SEQ ID NO: 95 depicted in FIG. 9A and selected from a group of: N18, N21, M22, R24, D25, S31, R32, D55, M68, 169, L73, E74, M77, P78, Q79, E81, N82, K88, A89, H90, N92, S93, G95, E96, N97, K99, T100, L101, L103, R104, R107, R110 and F111 (FIGS. 10A-11B). In some embodiments, the one or more amino acid substitutions are at position(s) selected from the group consisting of: N18, D28, N92, K99, and L103, numbering according to SEQ ID NO:95. In some embodiments, the one or more amino acid substitutions are selected from the group consisting of: N18F, N18L, N18Y, D28Q, D28R, N92F, N92H, N92I, N92K, N92L, N92R, N92S, N92T, N92V, N92Y, K99N, L103N, and L103Q, numbering according to SEQ ID NO:95. In yet other embodiments, the mutant IL-10 polypeptide exhibits increased binding affinity by 150% or more to IL-10RB polypeptide having an amino acid sequence as set forth by SEQ ID NO: 97 depicted in FIG. 9C.

The location of possible amino acid substitutions in the sequence of the wild-type mature IL-10 polypeptide is depicted in FIGS. 10A-10B. In some embodiments, denoted amino acids in the sequence of the wild-type mature IL-10 polypeptide were substituted for alanine or another amino acid, as depicted in FIGS. 11A-11B.

In some embodiments, the mutant IL-10 polypeptides also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell.

In some embodiments, the mutant IL-10 polypeptide further comprises an amino acid substitution relative to the amino acid sequence of SEQ ID NO:1 at position R107. In some embodiments, the mutant IL-10 polypeptide further comprises an R107A mutation, numbering according to SEQ ID NO:95.

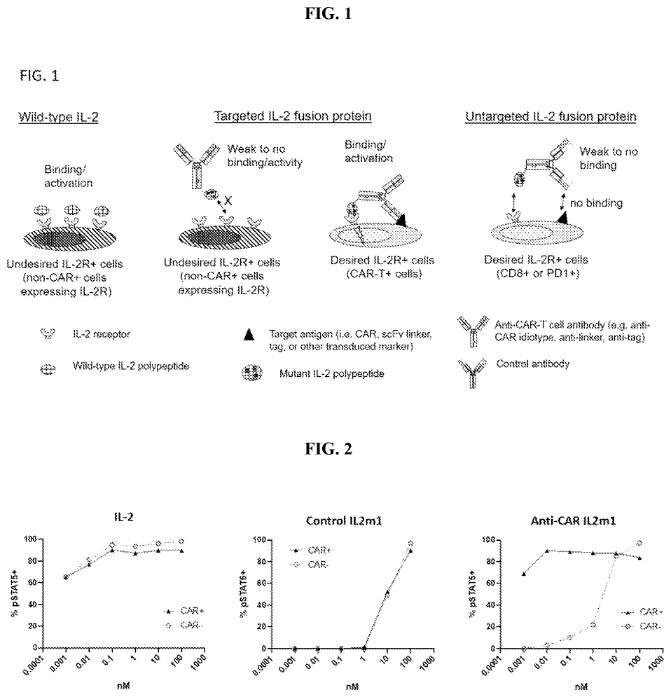

In some embodiments, the mutant IL-10 polypeptide is a monomer, e.g., comprising an amino acid or peptide insertion between N116 and K117 (e.g., as depicted in FIG. 9D) to enable folding and expression as a monomer. In some embodiments, the insertion is 1-15 amino acids in length. In some embodiments, the insertion is 1, 2, 3, 4, 5, 6, 7, 8, 9, 10, 11, 12, 13, 14, or 15 amino acids in length. In certain embodiments, the insertion is 6 amino acids in length. In some embodiments, the mutant IL-10 monomer polypeptide comprises the amino acid sequence of SEQ ID NO:95 with an amino acid or peptide insertion of between 1 and 15 amino acids immediately following residue C114, E115, N116, K117, S118, K119, or A120, numbering based on SEQ ID NO:95. Examples of insertion can include, without limitation, G, GG, GGG, GGGG, GGGSG, GGGGG, GGGGGG, and GGGSGG. In some embodiments, the mutant IL-10 monomer polypeptide comprises the amino acid sequence of SEQ ID NO:98. In some embodiments, the mutant monomer IL-10 polypeptides of the present disclosure have reduced binding affinity to IL-10RA polypeptide having an amino acid sequence depicted in FIG. 1B, and have amino acid substitutions selected from a group of: P20, L23, R24, R27, D28, K34, T35, Q38, M39, D41, L43, D44, N45, L46, K49, 187, V91, L94, L98, K138, S141, E142, D144, N148, E151, and 1158 (or selected from a group of: R24, R27, K34, Q38, D44, 187, K138, E142, D144, N148, and E151), where the amino acid numbering refers to the corresponding amino acids in the wild type IL-10 polypeptide without the 6 linker insertion. In some embodiments, the mutant monomer IL-10 polypeptides of the present disclosure also have increased binding affinity to IL-10RB polypeptide having an amino acid sequence depicted in FIG. 1C, and have amino acid substitutions selected from a group of: N18, N21, M22, R24, D25, D28, S31, R32, D55, M68, 169, L73, E74, M77, P78, Q79, E81, N82, K88, A89, H90, N92, S93, G95, E96, N97, K99, T100, L101, L103, R104, R107, R110 and F111 (or selected from a group of: N18, D28, N92, K99, and L103).

Table 3 depicts exemplary amino acid insertions and insertion positions for IL-10 monomer polypeptides of the present disclosure (insertions are underlined). In some embodiments, the mutant IL-10 monomer polypeptide comprises an amino acid sequence listed in Table 3. In some embodiments, the mutant IL-10 monomer polypeptide comprises an amino acid insertion as listed in Table 3 and/or at a position as listed in Table 3. In some embodiments, the insertion is 1, 2, 3, 4, 5, 6, 7, 8, 9, 10, 11, 12, 13, 14, or 15 amino acids in length. In some embodiments, the mutant IL-10 monomer polypeptide comprises the amino acid sequence of a mutant IL-10 monomer polypeptide of the present disclosure with an amino acid or peptide insertion of between 1 and 15 amino acids immediately following residue C114, E115, N116, K117, 5118, K119, or A120, numbering based on SEQ ID NO:95. Examples of insertion can include, without limitation, G, GG, GGG, GGGG, GGGSG, GGGGG, GGGGGG, and GGGSGG.

TABLE 3
Exemplary insertions and insertion positions of mutant monomer IL-10 polypeptides

| Construct | | |
|---|---|---|
| name | Amino Acid Sequence | SEQ ID NO. |
| IL10mono_ | LLKESLLEDFKGYLGCQALSEMIQFYLEEVMPQAENQDPDIKAHVN | 187 |
| insertC114_ | SPGQGTQSENSCTHFPGNLPNMLRDLRDAFSRVKTFFQMKDQLDNL | |
| E115 | SLGENLKTLRLRLRRCHRFLPCGGGSGGENKSKAVEQVKNAFNKLQ | |
| | EKGIYKAMSEFDIFINYIEAYMTMKIRN | |
| IL10mono_ | SPGQGTQSENSCTHFPGNLPNMLRDLRDAFSRVKTFFQMKDQLDNL | 189 |
| insertE115_ | LLKESLLEDFKGYLGCQALSEMIQFYLEEVMPQAENQDPDIKAHVN | |
| N116 | SLGENLKTLRLRLRRCHRELPCEGGGSGGNKSKAVEQVKNAFNKLQ | |
| | EKGIYKAMSEFDIFINYIEAYMTMKIRN | |
| IL10mono_ | SPGQGTQSENSCTHEPGNLPNMLRDLRDAFSRVKTFFQMKDQLDNL | 190 |
| insertK117_ | LLKESLLEDFKGYLGCQALSEMIQFYLEEVMPQAENQDPDIKAHVN | |
| S118 | SLGENLKTLRLRLRRCHRFLPCENKGGGSGGSKAVEQVKNAFNKLQ | |
| | EKGIYKAMSEFDIFINYIEAYMTMKIRN | |
| IL10mono_ | SPGQGTQSENSCTHEPGNLPNMLRDLRDAFSRVKTFFQMKDQLDNL | 191 |
| insertS118_ | LLKESLLEDFKGYLGCQALSEMIQFYLEEVMPQAENQDPDIKAHVN | |
| K119 | SLGENLKTLRLRLRRCHRFLPCENKSGGGSGGKAVEQVKNAFNKLQ | |
| | EKGIYKAMSEFDIFINYIEAYMTMKIRN | |
| IL10mono_ | LLKESLLEDFKGYLGCQALSEMIQFYLEEVMPQAENQDPDIKAHVN | 192 |
| insertK119_ | SPGQGTQSENSCTHEPGNLPNMLRDLRDAFSRVKTFFQMKDQLDNL | |
| A120 | SLGENLKTLRLRLRRCHRFLPCENKSKGGGSGGAVEQVKNAFNKLQ | |
| | EKGIYKAMSEFDIFINYIEAYMTMKIRN | |
| IL10mono_ | LLKESLLEDFKGYLGCQALSEMIQFYLEEVMPQAENQDPDIKAHVN | 193 |
| 1GinsertK117_ | SPGQGTQSENSCTHEPGNLPNMLRDLRDAFSRVKTFFQMKDQLDNL | |
| S118 | SLGENLKTLRLRLRRCHRFLPCENKGSKAVEQVKNAENKLQEKGIY | |
| | KAMSEFDIFINYIEAYMTMKIRN | |
| IL10mono_ | SPGQGTQSENSCTHEPGNLPNMLRDLRDAFSRVKTFFQMKDQLDNL | 194 |
| 2GinsertK117_ | LLKESLLEDFKGYLGCQALSEMIQFYLEEVMPQAENQDPDIKAHVN | |
| S118 | SLGENLKTLRLRLRRCHRFLPCENKGGSKAVEQVKNAFNKLQEKGI | |
| | YKAMSEFDIFINYIEAYMTMKIRN | |
| IL10mono_ | LLKESLLEDFKGYLGCQALSEMIQFYLEEVMPQAENQDPDIKAHVN | 195 |
| 3GinsertK117_ | SPGQGTQSENSCTHEPGNLPNMLRDLRDAFSRVKTFFQMKDQLDNL | |
| S118 | SLGENLKTLRLRLRRCHRFLPCENKGGGSKAVEQVKNAFNKLQEKG | |
| | IYKAMSEFDIFINYIEAYMTMKIRN | |
| IL10mono_ | LLKESLLEDFKGYLGCQALSEMIQFYLEEVMPQAENQDPDIKAHVN | 196 |
| 4GinsertK117_ | SPGQGTQSENSCTHFPGNLPNMLRDLRDAFSRVKTFFQMKDQLDNL | |
| S118 | SLGENLKTLRLRLRRCHRFLPCENKGGGGSKAVEQVKNAFNKLQEK | |
| | GIYKAMSEFDIFINYIEAYMTMKIRN | |
| IL10mono_ | LLKESLLEDFKGYLGCQALSEMIQFYLEEVMPQAENQDPDIKAHVN | 197 |
| 5GinsertK117_ | SPGQGTQSENSCTHFPGNLPNMLRDLRDAFSRVKTFFQMKDQLDNL | |
| S118 | SLGENLKTLRLRLRRCHRFLPCENKGGGGGSKAVEQVKNAENKLQE | |
| | KGIYKAMSEFDIFINYIEAYMTMKIRN | |
| IL10mono_ | LLKESLLEDFKGYLGCQALSEMIQFYLEEVMPQAENQDPDIKAHVN | 198 |
| 6GinsertK117_ | SPGQGTQSENSCTHFPGNLPNMLRDLRDAFSRVKTFFQMKDQLDNL | |
| S118 | SLGENLKTLRLRLRRCHRFLPCENKGGGGGGSKAVEQVKNAFNKLQ | |
| | EKGIYKAMSEFDIFINYIEAYMTMKIRN | |

In some embodiments, the mutant IL-10 polypeptide comprises an amino acid sequence selected from the group consisting of SEQ ID Nos: 99-112 as provided in Table 4.

TABLE 3
Amino acid sequences of exemplary mutant monomer IL-10 polypeptides.

| Construct | | |
|---|---|---|
| name | Amino Acid Sequence | SEQ ID NO. |
| IL10mono_ | SPGQGTQSENSCTHFPGNLPNMLADLADAFSRVKTFFQMKDQLDNL | 99 |
| RBenh2_m15 | LLKESLLEDFKGYLGCQALSEMIQFYLEEVMPQAENQDPDIKAHVI | |
| | SLGENLKTLRLRLRRCHRELPCENGGGSGGKSKAVEQVKNAFNKLQ | |
| | EKGIYKAMSEFDIFINYIEAYMTMKIRN | |
| IL10mono_ | SPGQGTQSENSCTHFPGNLPNMLRDLRDAFSRVKTFFQMKDQLDNL | 100 |
| RBenh2_m10 | LLKESLLEDFKGYLGCQALSEMIQFYLEEVMPQAENQDPDIKAHVI | |
| | SLGENLKTLRLRLRRCHRFLPCENGGGSGGKSKAVEQVKNAFNKLQ | |
| | EKGIYKAMSEFAIFINYIEAYMTMKIRN | |
| IL10mono_ | SPGQGTQSENSCTHEPGNLPNMLRDLRDAFSRVKTFFQMKDQLDNL | 101 |
| RBenh7_m10 | LLKESLLEDFKGYLGCQALSEMIQFYLEEVMPQAENQDPDIKAHVL | |
| | SLGENLKTLRLRLRRCHRFLPCENGGGSGGKSKAVEQVKNAFNKLQ | |
| | EKGIYKAMSEFAIFINYIEAYMTMKIRN | |
| IL10mono_ | SPGQGTQSENSCTHFPGNLPNMLRDLRDAFSRVKTFFQMKDQLDNL | 102 |
| RBenh2.1-m10 | LLKESLLEDFKGYLGCQALSEMIQFYLEEVMPQAENQDPDIKAHVI | |
| | SLGENLKTLRLRLRRCHRFLPCENKGGGSGGSKAVEQVKNAFNKLQ | |
| | EKGIYKAMSEFAIFINYIEAYMTMKIRN | |
| IL10mono_ | SPGQGTQSENSCTHEPGNLPNMLRDLRDAFSRVKTFFQMKDQLDNL | 103 |
| RBenh7.1-m10 | LLKESLLEDFKGYLGCQALSEMIQFYLEEVMPQAENQDPDIKAHVL | |
| | SLGENLKTLRLRLRRCHRFLPCENKGGGSGGSKAVEQVKNAFNKLQ | |
| | EKGIYKAMSEFAIFINYIEAYMTMKIRN | |
| IL10mono_ | SPGQGTQSENSCTHEPGNLPNMLADLADAFSRVKTFFQMKDQLDNL | 104 |
| RBenh2.1-m15 | LLKESLLEDFKGYLGCQALSEMIQFYLEEVMPQAENQDPDIKAHVI | |
| | SLGENLKTLRLRLRRCHRFLPCENKGGGSGGSKAVEQVKNAENKLQ | |
| | EKGIYKAMSEFDIFINYIEAYMTMKIRN | |
| IL10mono_ | SPGQGTQSENSCTHEPGNLPNMLRDLRDAFSRVKTFFQMKDQLDNL | 105 |
| RBenh2_m117 | LLKESLLEDFKGYLGCQALSEMIQFYLEEVMPQAENQDPDIKAHVI | |
| | SLGENLKTLRLRLRACHRELPCENGGGSGGKSKAVEQVKNAFNKLQ | |
| | EKGIYKAMSEFDIFINYIEAYMTMKIRN | |
| IL- | SPGQGTQSENSCTHEPGNLPNMLRDLRDAFSRVKTFFQMKDQLDNL | 106 |
| 10mono_ | LLKESLLEDFKGYLGCQALSEMIQFYLEEVMPQAENQDPDIKAHVI | |
| RBenh2-m10 | SLGENLKTLRLRLRACHRFLPCENGGGSGGKSKAVEQVKNAFNKLQ | |
| m117 | EKGIYKAMSEFAIFINYIEAYMTMKIRN | |
| IL- | LLKESLLEDFKGYLGCQALSEMIQFYLEEVMPQAENQDPDIKAHVL | 107 |
| 10mono_ | SPGQGTQSENSCTHFPGNLPNMLRDLRDAFSRVKTFFQMKDQLDNL | |
| RBenh7-m10 | SLGENLKTLRLRLRACHRELPCENGGGSGGKSKAVEQVKNAFNKLQ | |
| m117 | EKGIYKAMSEFAIFINYIEAYMTMKIRN | |
| IL- | SPGQGTQSENSCTHEPGNLPNMLADLADAFSRVKTFFQMKDQLDNL | 108 |
| 10mono_ | LLKESLLEDFKGYLGCQALSEMIQFYLEEVMPQAENQDPDIKAHVI | |
| RBenh2-m15 | SLGENLKTLRLRLRACHRFLPCENGGGSGGKSKAVEQVKNAENKLQ | |
| m117 | EKGIYKAMSEFDIFINYIEAYMTMKIRN | |
| IL- | SPGQGTQSENSCTHEPGNLPNMLRDLRDAFSRVKTFFQMKDQLDNL | 109 |
| 10mono_ | LLKESLLEDFKGYLGCQALSEMIQFYLEEVMPQAENQDPDIKAHVI | |
| RBenh2.1-m10 | SLGENLKTLRLRLRACHRELPCENKGGGSGGSKAVEQVKNAFNKLQ | |
| m117 | EKGIYKAMSEFAIFINYIEAYMTMKIRN | |
| IL- | LLKESLLEDFKGYLGCQALSEMIQFYLEEVMPQAENQDPDIKAHVL | 110 |
| 10mono_ | SPGQGTQSENSCTHFPGNLPNMLRDLRDAFSRVKTFFQMKDQLDNL | |
| RBenh7.1-m10 | SLGENLKTLRLRLRACHRFLPCENKGGGSGGSKAVEQVKNAFNKLQ | |
| m117 | EKGIYKAMSEFAIFINYIEAYMTMKIRN | |
| IL- | SPGQGTQSENSCTHFPGNLPNMLADLADAFSRVKTFFQMKDQLDNL | 111 |
| 10mono_ | LLKESLLEDFKGYLGCQALSEMIQFYLEEVMPQAENQDPDIKAHVI | |
| RBenh2.1-m15 | SLGENLKTLRLRLRACHRFLPCENKGGGSGGSKAVEQVKNAFNKLQ | |
| m117 | EKGIYKAMSEFDIFINYIEAYMTMKIRN | |
| IL10mono_ | SPGQGTQSENSCTHFPGNLPNMLRDLRDAFSRVKTFFQMKDQLDNL | 112 |
| RBenh2 | LLKESLLEDFKGYLGCQALSEMIQFYLEEVMPQAENQDPDIKAHVI | |
| | SLGENLKTLRLRLRRCHRFLPCENGGGSGGKSKAVEQVKNAFNKLQ | |
| | EKGIYKAMSEFDIFINYIEAYMTMKIRN | |

### Interleukin-7 (IL-7)

An example amino acid sequence for IL-7 polypeptide is provided below (SEQ ID NO: 91), wherein at least one of positions K10, Q11, S14, V15, V18, Q22, L35, N36, D74, L77, L80, K81, E84, 188, R133, Q136, E137, T140, and N143, and K144, can be mutated (e.g., K81A and T140A.

(SEQ ID NO: 91)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEH.

For instance, an exemplary mutant T1L-7 peptide comprises amino acid substitutions K81A and T140A. In some embodiments, the mutant T1L-7 peptide comprises the sequence of:

(SEQ ID NO: 113)
DCDIEGKDGKQYESVLMVSIDQLLDSMKEIGSNCLNNEFNFFKRHICDA
NKEGMFLFRAARKLRQFLKMNSTGDFDLHLLAVSEGTTILLNCTGQVKG
RKPAALGEAQPTKSLEENKSLKEQKKLNDLCFLKRLLQEIKACWNKILM
GTKEH.

In some embodiments, the cytokine is a mutant T1L-7 polypeptide that exhibits reduced binding affinity by 5000 or more to an IL-7Rα polypeptide comprising the amino acid sequence of SEQ ID NO: 94, compared to binding affinity of a wild-type T1L-7 polypeptide comprising the amino acid sequence of SEQ ID NO: 91 to the IL-7Rα polypeptide. In some embodiments, the mutant T1L-7 polypeptide exhibits reduced binding affinity by 5000 or more to an IL-7Rγ polypeptide comprising the amino acid sequence of SEQ ID NO: 4, compared to binding affinity of a wild-type T1L-7 polypeptide comprising the amino acid sequence of SEQ ID NO: 91 to the IL-7Rγ polypeptide.

In some embodiments, the IL-7Ra polypeptide comprises the sequence of:

(SEQ ID NO: 94)
MTILGTTFGMVFSLLQVVSGESGYAQNGDLEDAELDDYSFSCYSQLEVN
GSQHSLTCAFEDPDVNITNLEFEICGALVEVKCLNFRKLQEIYFIETKK
FLLIGKSNICVKVGEKSLTCKKIDLTTIVKPEAPFDLSVVYREGANDFV
VTFNTSHLQKKYVKVLMHDVAYRQEKDENKWTHVNLSSTKLTLLQRKLQ
PAAMYEIKVRSIPDHYFKGFWSEWSPSYYFRTPEINNSSGEMDPILLTI
SILSFFSVALLVILACVLWKKRIKPIVWPSLPDHKKTLEHLCKKPRKNL
NVSFNPESFLDCQIHRVDDIQARDEVEGFLQDTFPQQLEESEKQRLGGD
VQSPNCPSEDVVITPESFGRDSSLTCLAGNVSACDAPILSSSRSLDCRE
SGKNGPHVYQDLLLSLGTTNSTLPPPFSLQSGILTLNPVAQGQPILTSL
GSNQEEAYVTMSSFYQNQ.

### Interleukin-21 (IL-21)

In some embodiments, the cytokine is a mutant IL-21 polypeptide that exhibits reduced binding affinity by 50% or more to an IL-21Ry polypeptide comprising the amino acid sequence of SEQ ID NO: 93, compared to binding affinity of a wild-type IL-21 polypeptide comprising the amino acid sequence of SEQ ID NO: 92 or SEQ ID NO: 115 to the IL-21R polypeptide. In some embodiments, the mutant IL-21 polypeptide exhibits reduced binding affinity by 50% or more to an IL-21Ry polypeptide comprising the amino acid sequence of SEQ ID NO: 93, compared to binding affinity of a wild-type IL-21 polypeptide comprising the amino acid sequence of SEQ ID NO: 92 or SEQ ID NO: 115 to the IL-21Ry polypeptide.

“Interleukin-21” or “IL-21” as used interchangeably herein can refer to any native IL-21, unless otherwise indicated. “IL-21” can encompass unprocessed IL-21 (such as precursor IL-21) as well as “mature IL-21” which can be a form of IL-21 that results from processing in the cell. A sequence of human “mature IL-21” is provided as SEQ ID NO: 92 or SEQ ID NO: 115. “IL-21” can also include but is not limited to naturally occurring variants of IL-21, e.g., allelic or splice variants or variants. The amino acid sequence of an exemplary human IL-21 is described under UniProt Q9HBE4 (IL21_HUMAN). A “mutant IL-2 polypeptide” can refer to IL-2 polypeptide that can have altered affinity to its receptor, such as a reduced affinity to its receptor wherein such decreased affinity will result in reduced biological activity of the mutant. Alterations in affinity, such as reduction in affinity and thereby activity can be obtained by introducing a small number of amino acid mutations or substitutions. The mutant IL-21 polypeptides can also have other modifications to the peptide backbone, including but not limited to amino acid deletion, permutation, cyclization, disulfide bonds, or the post-translational modifications (e.g., glycosylation or altered carbohydrate) of a polypeptide, chemical or enzymatic modifications to the polypeptide (e.g., attaching PEG to the polypeptide backbone), addition of peptide tags or labels, or fusion to proteins or protein domains to generate a final construct with desired characteristics, such as reduced affinity to IL-21R. Desired activity may also include improved biophysical properties compared to the wild-type IL-21 polypeptide. Multiple modifications may be combined to achieve a desired activity modification, such as reduction or increase in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. In some cases, a mutation or mutations are introduced to the polypeptide to modify its activity.

IL-21 has a four-helix bundle structure and exists as a monomer. In humans, two isoforms of IL-21 are known, each of which are derived from a precursor molecule. The first IL-21 isoform comprises 162 amino acids (aa), the first 29 of which make up the signal peptide; and the second IL-21 isoform comprises 153 aa, the first 29 of which make up the signal peptide as in the first isoform.

IL-21 binds to the heterodimeric IL-21 receptor complex, comprising of an IL-21 receptor (IL-21R) and common gamma chain (γc). IL-21 receptor complex is expressed on the surface of T, B, and NK cells. IL-21 receptor complex is similar in structure to the IL-2 receptor complex, in that each of these cytokine receptor complex comprises a γc.

When IL-21 binds to IL-21 receptor complex, the JAK/STAT signaling pathway is activated to activate target genes. While IL-21-induced signaling may be therapeutically desirable, careful consideration of the timing and the location of the signaling is needed, given IL-21's broad expression profile and due to the fact that IL-21 has the ability to potentiate CD8+ T cell responses as well as to suppress antigen presentation and T cell priming.

In some embodiments, the IL-21 comprises the sequence of:

(SEQ ID NO: 92)
HKSSSQGQDRHMIRMRQLIDIVDQLKNYVNDLVPEFLPAPEDVETNCEW
SAFSCFQKAQLKSANTGNNERIINVSIKKLKRKPPSTNAGRRQKHRLTC
PSCDSYEKKPPKEFLERFKSLLQKMIHQHLSSRTHGSEDS.

In some embodiments, the IL-21 comprises the sequence of:

(SEQ ID NO: 115)
QGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDS
YEKKPPKEFLERFKSLLQKMIHQHLSSRTHGSEDS.

In some embodiments, the IL-21R polypeptide comprises the sequence of:

(SEQ ID NO: 93)
MPRGWAAPLLLLLLQGGWGCPDLVCYTDYLQTVICILEMWNLHPSTLTL
TWQDQYEELKDEATSCSLHRSAHNATHATYTCHMDVFHFMADDIFSVNI
TDQSGNYSQECGSFLLAESIKPAPPFNVTVTFSGQYNISWRSDYEDPAF
YMLKGKLQYELQYRNRGDPWAVSPRRKLISVDSRSVSLLPLEFRKDSSY
ELQVRAGPMPGSSYQGTWSEWSDPVIFQTQSEELKEGWNPHLLLLLLLV
IVFIPAFWSLKTHPLWRLWKKIWAVPSPERFFMPLYKGCSGDFKKWVGA
PFTGSSLELGPWSPEVPSTLEVYSCHPPRSPAKRLQLTELQEPAELVES
DGVPKPSFWPTAQNSGGSAYSEERDRPYGLVSIDTVTVLDAEGPCTWPC
SCEDDGYPALDLDAGLEPSPGLEDPLLDAGTTVLSCGCVSAGSPGLGGP
LGSLLDRLKPPLADGEDWAGGLPWGGRSPGGVSESEAGSPLAGLDMDTF
DSGFVGSDCSSPVECDFTSPGDEGPPRSYLRQWVVIPPPLSSPGPQAS.

The present disclosure provides, in some embodiments, IL-21 polypeptides or functional fragments or variants thereof, comprising at least one amino acid substitution, relative to the wild-type IL-21 amino acid sequence, which is provided herein as SEQ ID NO: 92 or SEQ ID NO: 115. Such IL-21 polypeptides comprising at least one amino acid substitution relative to SEQ ID NO: 92 or SEQ ID NO: 115 are also referred to herein as IL-21 muteins. In exemplary aspects, an IL-21 polypeptide or functional fragment or variant thereof, as described herein, comprises at least one and not more than X amino acid substitutions, wherein X is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or greater. In some embodiments, an IL-21 polypeptide or functional fragment or variant thereof, as described herein, comprises at least 35 amino acid substitutions compared to SEQ ID NO: 115. In exemplary embodiments, an IL-21 polypeptide or functional fragment or variant thereof, as described herein, comprises an amino acid sequence which differs from the amino acid sequence of human IL-21 (e.g., SEQ ID NO: 115) by 10 amino acids, 15 amino acids, 20 amino acids, or 25 amino acids. In exemplary embodiments, an IL-21 polypeptide or functional fragment or variant thereof, as described herein, comprises an amino acid sequence which differs from the amino acid sequence of human IL-21 (e.g., SEQ ID NO: 115) by no more than 7 amino acids or no more than 5 amino acids. In exemplary embodiments, an IL-21 polypeptide or functional fragment or variant thereof, as described herein, comprises an amino acid sequence which differs from the amino acid sequence of human IL-21 (e.g., SEQ ID NO: 115) by 3, 4, 5, or 6 amino acids. In exemplary embodiments, an IL-21 polypeptide or functional fragment or variant thereof, as described herein, comprises an amino acid sequence which differs from the amino acid sequence of human IL-21 (e.g., SEQ ID NO: 115) by 3 to 6 amino acids or 1 to 5 amino acids. In exemplary embodiments, an IL-21 polypeptide or functional fragment or variant thereof, as described herein, comprises an amino acid sequence which differs from the amino acid sequence of human IL-21 (e.g., SEQ ID NO: 115) by one or two amino acids.

### Reduced IL-21R Binding

In some embodiments, an IL-21 polypeptide or a functional fragment or a variant thereof comprises at least one mutation that reduces its binding affinity to IL-21R. Such mutations are in some embodiments in one or more positions selected from the group consisting of: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115.

In some embodiments, the mutation at position R5 comprises an amino acid substitution selected from the group consisting of A, D, E, S, T, N, Q, V, I, L, Y, or F. In some embodiments, the mutation at position 18 comprises an amino acid substitution selected from the group consisting of Q, H, E. In some embodiments, the mutation at position R9 comprises an amino acid substitution selected from the group consisting of A, D, E, S, T, N, Q, V, I, L, Y, or F. In some embodiments, the mutation at position R11 comprises an amino acid substitution selected from the group consisting of D or E. In some embodiments, the mutation at position Q12 comprises an amino acid substitution selected from the group consisting of L, I, or Y. In some embodiments, the mutation at position 114 comprises an amino acid substitution selected from the group consisting of D or E. In some embodiments, the mutation at position D15 comprises an amino acid substitution selected from the group consisting of R, K, H, L, Y, or F. In some embodiments, the mutation at position D18 comprises an amino acid substitution selected from the group consisting of A, K, or R. In some embodiments, the mutation at position Q19 comprises an amino acid substitution selected from the group consisting of L, or Y. In some embodiments, the mutation at position Y23 comprises an amino acid substitution of E. In some embodiments, the mutation at position R65 comprises an amino acid substitution selected from the group consisting of G, S, E, D or A. In some embodiments, the mutation at position S70 comprises an amino acid substitution selected from the group consisting of H, Y, L, V or F. In some embodiments, the mutation at position K72 comprises an amino acid substitution selected from the group consisting of G, S, E, D or A. In some embodiments, the mutation at position K73 comprises an amino acid substitution selected from the group consisting of A, Y, L, F, G, S, T, E, or D. In some embodiments, the mutation at position K75 comprises an amino acid substitution selected from the group consisting of G, S, E, D or A. In some embodiments, the mutation at position R76 comprises an amino acid substitution selected from the group consisting of A, D, E, S, T, N, Q, V, I, L, Y, or F. In some embodiments, the mutation at position K77 comprises an amino acid substitution selected from the group consisting of G, S, E, D or A. In some embodiments, the mutation at position S80 comprises an amino acid substitution of H, A, G, E, or D. In some embodiments, the mutation at position Q116 comprises an amino acid substitution is Y. In some embodiments, the mutation at position K117 comprises an amino acid substitution selected from the group consisting of A, D, or E.

An example sequence for an IL-21 polypeptide or a functional fragment or a variant thereof of this disclosure is provided as follows: QGQDX₁HMX₂X₃MX₄X5LX₆X₇IVX₈X₉LKNX₁₀VNDLVPEFLPAPEDVETNCEWSAFSCFQK AQLKSANTGNNEX₁₁IINVX₁₂IX₁₃X₁₄LX₁₅X₁₆X₁₇PPX₁₈TNAGRRQKHRLTCPSCDSYEKKP PKEFLERFKSLLX₁₉X₂₀MIHQHLSSRTHGSEDS (SEQ ID NO: 136). In some embodiments, X₁=R, A, D, E, S, T, N, Q, V, I, L, Y, or F. In some embodiments, X₂=I, Q, H, E. In some embodiments, X₃=R, A, D, E, S, T, N, Q, V, I, L, Y, or F. In some embodiments, X₄=R, D or E. In some embodiments, X₅=Q, L, I, or Y. In some embodiments, X₆=I, D or E. In some embodiments, X₇=D, R, K, H, L, Y, or F. In some embodiments, X₈=D, A, K, or R. In some embodiments, X₉=Q, L, or Y. In some embodiments, X₁₀=Y or E. In some embodiments, X₁₁=R, G, S, E, D, or A. In some embodiments, X12=S, H, Y, L, V, or F. In some embodiments, X13=K, G, S, E, D, or A. In some embodiments, X₁₄=K, A, Y, L, F, G, S, T, E, A, or D. In some embodiments, X₁₅=K, G, S, E, D, or A. In some embodiments, X₁₆=R, A, D, E, S, T, N, Q, V, I, L, Y, or F. In some embodiments, X₁₇=K, G, S, E, D, or A. In some embodiments, X₁₈=S, H, A, G, E, or D. In some embodiments, X₁₉=Q or Y. In some embodiments, X₂₀=K, A, D, or E.

TABLE 5
Exemplary IL-21 Sequences

| SEQ ID | | |
|---|---|---|
| NO: | Name | Sequence |
| 115 | Wild-Type | QGQDRHMIRMRQLIDIVDQLKNYVNDLVPEFLPAPEDVETNCEW |
| | IL-21 | SAFSCFQKAQLKSANTGNNERIINVSIKKLKRKPPSTNAGRROK |
| | | HRLTCPSCDSYEKKPPKEFLERFKSLLQKMIHQHLSSRTHGSED |
| | | S |
| 136 | Exemplary | QGQDX₁HMX₂X₃MX₄X₅LX₆X₇IVX₈X₉LKNX₁₀VNDLVPEFLPAPED |
| | IL-21 | VETNCEWSAFSCFQKAQLKSANTGNNEX₁₁IINVX₁₂IX₁₃X₁₄LX₁₅ |
| | scaffold | X₁₆X₁₇PPX₁₈TNAGRRQKHRLTCPSCDSYEKKPPKEFLERFKSLL |
| | sequence | X₁₉X₂₀MIHQHLSSRTHGSEDS |

Certain aspects of the present disclosure relate to methods of treating cancer or chronic infection. In some embodiments, the methods comprise administering an effective amount of a targeted cytokine construct, or a pharmaceutical composition comprising the targeted and a pharmaceutically acceptable carrier, to a patient. In some embodiments, the patient in need of said treatment has been diagnosed with cancer.

In some embodiments, the targeted cytokine construct or composition is administered in combination with an engineered cell therapy (e.g. a T cell therapy), a cancer vaccine, chemotherapeutic agent, or immune checkpoint inhibitor (ICI). In some embodiments, the chemotherapeutic agent is a kinase inhibitor, antimetabolite, cytotoxin or cytostatic agent, anti-hormonal agent, platinum-based chemotherapeutic agent, methyltransferase inhibitor, antibody, or anti-cancer peptide. In some embodiments, the immune checkpoint inhibitor targets PD-L1, PD-1, CTLA-4, CEACAM, LAIR1, CD160, 2B4, CD80, CD86, CD276, VTCN1, HVEM, KIR, A2AR, MHC class I, MHC class II, GALS, adenosine, TGFR, OX40, CD137, CD40, IDO, CSF1R, TIM-3, BTLA, VISTA, LAG-3, TIGIT, IDO, MICA/B, LILRB4, SIGLEC-15, or arginase, including without limitation an inhibitor of PD-1 (e.g., an anti-PD-1 antibody), PD-L1 (e.g., an anti-PD-L1 antibody), or CTLA-4 (e.g., an anti-CTLA-4 antibody). Examples of T cell therapies include, without limitation, CD4+ or CD8+ T cell-based therapies, adoptive T cell therapies, chimeric antigen receptor (CAR)-based T cell therapies, tumor-infiltrating lymphocyte (TIL)-based therapies, autologous T cell therapies, and allogeneic T cell therapies. Exemplary cancer vaccines include, without limitation, dendritic cell vaccines, vaccines comprising one or more polynucleotides encoding one or more cancer antigens, and vaccines comprising one or more cancer antigenic peptides.

In some embodiments, a targeted cytokine construct of the present disclosure is part of a pharmaceutical composition, e.g., including the targeted cytokine construct and one or more pharmaceutically acceptable carriers. Pharmaceutical compositions and formulations as described herein can be prepared by mixing the active ingredients (such as a targeted cytokine construct) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In some embodiments, a targeted cytokine construct of the present disclosure is lyophilized.

### Engineered Cell Therapy

In some embodiments, the present disclosure relates to administering an engineered cell therapy, e.g., an engineered cell therapy, to a subject in need thereof. Engineered cell therapy can be a type of immunotherapy in which cells (e.g., T cells) are administered to a subject to help the body fight diseases, such as cancer. In cancer therapy, T cells are usually taken from the patient's own blood or tumor tissue, grown in large numbers in the laboratory, and then given back to the patient to help the immune system fight the cancer. Sometimes, the T cells are changed in the laboratory, to generate engineered cells, that have improved ability to target the subject's cancer cells and kill them. Types of engineered cell therapy can include, but not limited to, chimeric antigen receptor T cell (CAR T-cell) therapy, T cell receptor (TCR) therapy, and tumor-infiltrating lymphocyte (TIL) therapy.

In some embodiments, an engineered cell therapy for use in a combination therapy as described herein includes administering engineered cells expressing recombinant receptors designed to recognize and/or specifically bind to molecules associated with the disease or condition and result in a response, such as an immune response against such molecules upon binding to such molecules. The receptors can include chimeric receptors, e.g., chimeric antigen receptors (CARs), and other transgenic antigen receptors including transgenic T cell receptors (TCRs). In some embodiments, T cells having endogenous T cell receptors that recognize and/or specifically bind to molecules associated with the disease or condition are isolated for the T cell therapy.

In some embodiments, the cells contain or are engineered to contain a receptor, e.g., an engineered antigen receptor, such as a chimeric antigen receptor (CAR) or a T cell receptor (TCR). Also provided are populations of such cells, compositions containing such cells and/or enriched for such cells, such as in which cells of a certain type such as T cells or CD8+ or CD4+ cells are enriched or selected. Also provided are therapeutic methods for administering the cells and compositions to subjects, e.g., patients. In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some embodiments, the cells used in the cell therapy described herein can express receptors, such as native or recombinant receptors, such as antigen receptors including functional non-TCR antigen receptors, e.g., chimeric antigen receptors (CARs), and other antigen-binding receptors such as transgenic T cell receptors (TCRs). Also among the receptors can be other chimeric receptors. In some embodiments, the cells can express receptors specific for the cytokine of a targeted cytokine construct as described herein.

### Chimeric Antigen Receptor (CAR)

Exemplary antigen receptors, including CARs, exemplary CAR-T cells, and methods for engineering and introducing such receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al, Cancer Discov., 3(4): 388-398 (2013); Davila et al, PLoS ONE 8(4): e61338 (2013); Turtle et al, Curr. Opin. Immunol, 24(5): 633-39 (2012); Wu et al, Cancer, 18(2): 160-75 (2012). In some aspects, the antigen receptors include a CAR as described in U.S. Pat. No. 7,446,190, and those described in International Patent Application Publication No. WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al, Nature Reviews Clinical Oncology, 10, 267-276 (2013); Wang et al, J. Immunother. 35(9): 689-701 (2012); and Brentjens et al, Sci Transl Med. 5(177) (2013). See also WO2014031687, U.S. Pat. Nos. 8,339,645, 7,446,179, US 2013/0149337, U.S. Pat. Nos. 7,446,190, and 8,389,282. The chimeric receptors, such as CARs, can include an extracellular antigen binding domain, such as a portion of an antibody molecule, generally a variable heavy (V_{H}) chain region and/or variable light (V_{L}) chain region of the antibody, e.g., an scFv antibody fragment. Non-limiting examples of CARs or CAR-T cells that can be used in the methods or compositions provided herein include tisagenlecleucel, axicabtagene ciloleucel, lisocabtagene maraleucel, and CAR-T cells engineered to target antigens like CD19, CD22, WT1, CD171 (LlCAM), MUC16, ROR1, or Lewis Y (LeY) blood group antigen.

An antigen binding domain of a chimeric transmembrane receptor polypeptide can comprise any protein or molecule that can bind to an antigen. An antigen binding domain of a chimeric transmembrane receptor polypeptide disclosed herein can be a monoclonal antibody, a polyclonal antibody, a recombinant antibody, a human antibody, a humanized antibody, or a functional derivative, variant or fragment thereof, including, but not limited to, a Fab, a Fab′, a F(ab′)2, an Fv, a single-chain Fv (scFv), minibody, a diabody, and a single-domain antibody such as a heavy chain variable domain (VH), a light chain variable domain (VL) and a variable domain (VHH) of camelid derived Nanobody. In some embodiments, an antigen binding domain comprises at least one of a Fab, a Fab′, a F(ab′)₂, an Fv, and a scFv. In some embodiments, an antigen binding domain comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding domain comprises a transmembrane receptor, or any derivative, variant, or fragment thereof. For example, an antigen binding domain can comprise at least a ligand binding domain of a transmembrane receptor.

In some embodiments, the antigen binding domain comprises a humanized antibody. A humanized antibody can be produced using a variety of techniques including, but not limited to, CDR-grafting, veneering or resurfacing, chain shuffling, and other techniques. Human variable domains, including light and heavy chains, can be selected to reduce the immunogenicity of humanized antibodies. In some embodiments, the antigen binding domain of a chimeric transmembrane receptor polypeptide comprises a fragment of a humanized antibody which binds an antigen with high affinity and possesses other favorable biological properties, such as reduced and/or minimal immunogenicity. A humanized antibody or antibody fragment can retain a similar antigenic specificity as the corresponding non-humanized antibody.

In some embodiments, the antigen binding domain comprises a single-chain variable fragment (scFv). scFv molecules can be produced by linking the heavy chain (V_{H}) and light chain (V_{L}) regions of immunoglobulins together using flexible linkers, such as polypeptide linkers. scFvs can be prepared according to various methods.

In some embodiments, the antigen binding domain is engineered to bind a specific target antigen. For example, the antigen binding domain can be an engineered scFv. An antigen binding domain comprising a scFv can be engineered using a variety of methods, including but not limited to display libraries such as phage display libraries, yeast display libraries, cell based display libraries (e.g., mammalian cells), protein-nucleic acid fusions, ribosome display libraries, and/or an E. coli periplasmic display libraries. In some embodiments, an antigen binding domain which is engineered may bind to an antigen with a higher affinity than an analogous antibody or an antibody which has not undergone engineering.

In some embodiments, the antigen binding domain binds multiple antigens, e.g., at least 2, 3, 4, 5, 6, 7, 8, 9, or 10 antigens. An antigen binding domain can bind two related antigens, such as two subtypes of botulin toxin (e.g., botulinum neurotoxin subtype A1 and subtype A2). An antigen binding domain can bind two unrelated proteins, such as receptor tyrosine kinase erbB-2 (also referred to as Neu, ERBB2, and HER2) and vascular endothelial growth factor (VEGF). An antigen binding domain capable of binding two antigens can comprise an antibody engineered to bind two unrelated protein targets at distinct but overlapping sites of the antibody. In some embodiments, an antigen binding domain which binds multiple antigens comprises a bispecific antibody molecule. A bispecific antibody molecule can have a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). The first and second epitopes can overlap. In some embodiments, the first and second epitopes do not overlap. In some embodiments, the first and second epitopes are on different antigens, e.g., different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope.

In some embodiments, the extracellular region of a chimeric transmembrane receptor polypeptide comprises multiple antigen binding domains, for example at least 2 antigen binding domains (e.g., at least 3, 4, 5, 6, 7, 8, 9, or 10 antigen binding domains). The multiple antigen binding domains can exhibit binding to the same or different antigen. In some embodiments, the extracellular region comprises at least two antigen binding domains, for example at least two scFvs linked in tandem. In some embodiments, two scFv fragments are linked by a peptide linker.

In some embodiments, the antigen that the chimeric antigen receptor (CAR) can bind to is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen binding domain expressed on the engineered cell binds an antigen selected from the group consisting of: a neoepitope from a tumor-associated antigen, TSHR, CD19, CD123, CD22, CD30, CD171, CS-1, CLL-1, CD33, EGFRvlll, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11 Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, ber-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51 E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1 B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, 1-40-β-amyloid, 4-1BB, 5AC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (a chain of IL-2receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSF1R, CSF2, CTLA-4, C-X-C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-7, IgE, IgE Fc region, IGF-1 receptor, IGF-1, IGHE, IL 17A, IL 17F, IL 20, IL-12, IL-13, IL-17, IL-1, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7 β7, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-R α, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β 1, TGF-β 2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRP1 (glycoprotein 75), VEGFA, VEGFR1, VEGFR2, vimentin, and VWF.

In some embodiments, the antigen that the chimeric antigen receptor (CAR) can bind to is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Ra2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and x-light chain. In some embodiments, the antigen binding domain binds to a tumor associated antigen.

In some embodiments, the antigen binding domain binds to an antibody e.g., an antibody bound to a cell surface protein or polypeptide. The protein or polypeptide on the cell surface bound by an antibody can comprise an antigen associated with a disease such as a viral, bacterial, and/or parasitic infection; inflammatory and/or autoimmune disease; or neoplasm such as a cancer and/or tumor. In some embodiments, the antibody binds a tumor associated antigen (e.g., protein or polypeptide). In some embodiments, an antigen binding domain of a chimeric transmembrane receptor polypeptide disclosed herein can bind a monoclonal antibody, a polyclonal antibody, a recombinant antibody, a human antibody, a humanized antibody, or a functional derivative, variant or fragment thereof, including, but not limited to, a Fab, a Fab′, a F(ab′)2, an Fc, an Fv, a scFv, minibody, a diabody, and a single-domain antibody such as a heavy chain variable domain (VH), a light chain variable domain (VL) and a variable domain (VHH) of camelid derived Nanobody. In some embodiments, an antigen binding domain can bind at least one of a Fab, a Fab′, a F(ab′)₂, an Fc, an Fv, and a scFv. In some embodiments, the antigen binding domain binds an Fc domain of an antibody.

In some embodiments, the antigen binding domain binds to an antibody selected from the group consisting of: 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, ABP 494 (cetuximab biosimilar), abrilumab, ABT-700, ABT-806, Actimab-A (actinium Ac-225 lintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch. 14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hul4.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imeiromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDIO680, MEDI-3617, MEDI-551 (inebilizumab), MEDI-565, MEDI6469, mepolizumab, metelimumab, MGB453, MGD006/S80880, MGD007, MGD009, MGDO11, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, R07009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox. In certain embodiments, the antigen binding domain binds an Fe domain of an aforementioned antibody.

In some embodiments, the antigen binding domain binds to an antibody mimetic. Antibody mimetics, as described elsewhere herein, can bind a target molecule with an affinity comparable to an antibody. In some embodiments, the antigen binding domain can bind a humanized antibody which is described elsewhere herein. In some embodiments, the antigen binding domain of a chimeric transmembrane receptor polypeptide can bind a fragment of a humanized antibody. In some embodiments, the antigen binding domain can bind a single-chain variable fragment (scFv).

In some embodiments, the antigen binding domain also binds to an Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A and protein G). Additionally, Fc binding domains may be synthetic polypeptides engineered specifically to bind the Fc portion of any of the Ig molecules described herein with desired affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, and a nanobody. Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide library for binding activities to Fc.

In some embodiments, the antigen binding domain comprises an Fc binding domain comprising an extracellular ligand-binding domain of a mammalian Fc receptor. Fc receptors are generally cell surface receptors expressed on the surface of many immune cells (including B cells, dendritic cells, natural killer (NK) cells, macrophages, neutrophils, mast cells, and eosinophils) and exhibit binding specificity to the Fc domain of an antibody. In some cases, binding of an Fc receptor to an Fc portion of the antibody can trigger antibody dependent cell-mediated cytotoxicity (ADCC) effects. The Fc receptor used for constructing a chimeric transmembrane receptor polypeptide described herein may be a naturally-occurring polymorphism variant, such as a variant which may have altered (e.g., increased or decreased) affinity to an Fc domain as compared to a wild-type counterpart. Alternatively, the Fc receptor may be a functional variant of a wild-type counterpart, carrying one or more mutations (e.g., up to 10 amino acid residue substitutions) that alters the binding affinity to the Fc portion of an Ig molecule. In some embodiments, the mutation may alter the glycosylation pattern of the Fc receptor and thus the binding affinity to an Fc domain.

Fc receptors can be classified based on the isotype of the antibody to which it is able to bind. For example, Fc-gamma receptors (FcγR) generally bind to IgG antibodies (e.g., IgG1, IgG2, IgG3, and IgG4); Fc-alpha receptors (FcαR) generally bind to IgA antibodies; and Fc-epsilon receptors (FcεR) generally bind to IgE antibodies. In some embodiments, the antigen binding domain comprises an Fc7 receptor or any derivative, variant or fragment thereof. In some embodiments, the antigen binding domain comprises an Fc binding domain comprising an FcR selected from FcγRI (CD64), FcγRIa, FcγRIb, FcγRIc, FcγRIIA (CD32) including allotypes H131 and R131, FcγRIIB (CD32) including FcγRIIB-1 and FcγRIIB-2, FcγRIIIA (CD16a) including allotypes V158 and F158, FcγRIIIB (CD16b) including allotypes FcγRIIIb-NAl and FcγRIIIb-NA2, any derivative thereof, any variant thereof, and any fragment thereof. An FcγR may be from any organism, including but not limited to humans, mice, rats, rabbits, and monkeys. Mouse FcγRs include but are not limited to FcγRI (CD64), FcγRII (CD32), FcδRIII (CD16), and FcγRIII-2 (CD16-2). In some embodiments, the antigen binding domain comprises an Fcε receptor or any derivative, variant or fragment thereof. In some embodiments, the antigen binding domain comprises a FcR selected from FcεRI, FcεRII (CD23), any derivative thereof, any variant thereof, and any fragment thereof. In some embodiments, the antigen binding domain comprises an Fcα receptor or any derivative, variant or fragment thereof. In some embodiments, the antigen binding domain comprises an FcR selected from FcαRI (CD89), Fcα/μR, any derivative thereof, any variant thereof, and any fragment thereof. In some embodiments, the antigen binding domain comprises an FcR selected from FcRn, any derivative thereof, any variant thereof, and any fragment thereof. Selection of the ligand binding domain of an Fc receptor for use in the chimeric transmembrane receptor polypeptides may depend on various factors such as the isotype of the antibody to which binding of the Fc binding domain is desired and the desired affinity of the binding interaction.

An immune cell signaling domain of an intracellular region of a chimeric transmembrane receptor polypeptide of a subject system can comprise a primary signaling domain. A primary signaling domain can be any signaling domain, or derivative, variant or fragment thereof, involved in immune cell signaling. For example, a signaling domain is involved in regulating primary activation of the TCR complex either in a stimulatory way or in an inhibitory way. An primary signaling domain can comprise a signaling domain of an Fcγ receptor (FcγR), an Fcε receptor (FcεR), an Fcα receptor (FcαR), neonatal Fc receptor (FcRn), CD3, CD3 ζ, CD3 γ, CD3 δ, CD3 ε, CD4, CD5, CD8, CD21, CD22, CD28, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70. In some embodiments, the primary signaling domain comprises an immunoreceptor tyrosine-based activation motif or ITAM. A primary signaling domain comprising an ITAM can comprise two repeats of the amino acid sequence YxxL/I separated by 6-8 amino acids, wherein each x is independently any amino acid, producing the conserved motif YxxL/Ix₍₆₋₈₎YxxL/I. A primary signaling domain comprising an ITAM can be modified, for example, by phosphorylation when the antigen binding domain is bound to an antigen. A phosphorylated ITAM can function as a docking site for other proteins, for example proteins involved in various signaling pathways. In some embodiments, the primary signaling domain comprises a modified ITAM domain, e.g., a mutated, truncated, and/or optimized ITAM domain, which has altered (e.g., increased or decreased) activity compared to the native ITAM domain.

In some embodiments, the primary signaling domain comprises an FcγR signaling domain (e.g., ITAM). The FcγR signaling domain can be selected from FcγRI (CD64), FcγRIIA (CD32), FcγRII1B (CD32), FcγRIIIA (CD16a), and FcγRIII1B (CD16b). In some embodiments, the primary signaling domain comprises an FcεR signaling domain (e.g., ITAM). The FcεR signaling domain can be selected from FcεRI and FcεRII (CD23). In some embodiments, the primary signaling domain comprises an FcαR signaling domain (e.g., ITAM). The FcαR signaling domain can be selected from FcαRI (CD89) and Fcα/μR. In some embodiments, the primary signaling domain comprises a CD3 ζ signaling domain. In some embodiments, the primary signaling domain comprises an ITAM of CD3 ζ.

In some embodiments, a primary signaling domain comprises an immunoreceptor tyrosine-based inhibition motif or ITIM. A primary signaling domain comprising an ITIM can comprise a conserved sequence of amino acids (S/I/V/LxYxxI/V/L) that is found in the cytoplasmic tails of some inhibitory receptors of the immune system. A primary signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A primary signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1 (IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LTR8), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PTLR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLECS or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some embodiments, the primary signaling domain comprises a modified ITIM domain, e.g., a mutated, truncated, and/or optimized ITIM domain, which has altered (e.g., increased or decreased) activity compared to the native ITIM domain.

In some embodiments, the immune cell signaling domain comprises multiple primary signaling domains. For example, the immune cell signaling domain can comprise at least 2 primary signaling domains, e.g., at least 2, 3, 4, 5, 7, 8, 9, or 10 primary signaling domains. In some embodiments, the immune cell signaling domain comprises at least 2 ITAM domains (e.g., at least 3, 4, 5, 6, 7, 8, 9, or 10 ITAM domains). In some embodiments, the immune cell signaling domain comprises at least 2 ITIM domains (e.g., at least 3, 4, 5, 6, 7, 8, 9, or 10 ITIM domains) (e.g., at least 2 primary signaling domains). In some embodiments, the immune cell signaling domain comprises both ITAM and ITIM domains.

The immune cell signaling domain of an intracellular region of a chimeric transmembrane receptor polypeptide can include a co-stimulatory domain. In some embodiments, a co-stimulatory domain, for example from co-stimulatory molecule, can provide co-stimulatory signals for immune cell signaling, such as signaling from ITAM and/or ITIM domains, e.g., for the activation and/or deactivation of immune cells. In some embodiments, an immune cell signaling domain comprises a primary signaling domain and at least one co-stimulatory domain. In some embodiments, a costimulatory domain is operable to regulate a proliferative and/or survival signal in the immune cell. In some embodiments, a co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the co-stimulatory domain comprises a signaling domain of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSFIB, TNF-α, TRANCE/RANKL, TSLP, TSLP R, VLA1, and VLA-6. In some embodiments, the immune cell signaling domain comprises multiple co-stimulatory domains, for example at least two, e.g., at least 3, 4, or 5 co-stimulatory domains.

### T Cell Receptor (TCR)

In some embodiments, in an engineered cell therapy, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. TCRs that exist in αβ and γδ forms can be structurally similar, but T cells expressing them can have distinct anatomical locations or functions. A TCR can be found on the surface of a cell or in soluble form.

A TCR can be found on the surface of T cells (or T lymphocytes) where it can be responsible for recognizing antigens bound to major histocompatibility complex (MHC) molecules.

Unless otherwise stated, the term “TCR” is understood to encompass full TCRs as well as antigen-binding portions or antigen-binding fragments thereof. In some embodiments, the TCR is an intact or full-length TCR, including TCRs in the αβ form or 76 form. In some embodiments, an αβT cells are redirected against cancer cells by transferring into the αβT cells a broadly tumor-reactive 76T-cell receptor, for instance, γ9δ2TCR-transduced αβT cells. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR contains only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable β chain of a TCR, sufficient to form a binding site for binding to a specific MHC-peptide complex. The variable chains of a TCR can contain complementarity determining regions involved in recognition of the peptide, MHC and/or MHC-peptide complex.

In some embodiments, the variable domains of the TCR contain hypervariable loops, or complementarity determining regions (CDRs), which can be the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain can be separated by framework regions (FRs), which can display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al, Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some cases, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the j-chain contain a further hypervariable region (CDR4 or HVR4), which can be involved in superantigen binding and not antigen recognition.

In some embodiments, a TCR also contains a constant domain, a transmembrane domain and/or a short cytoplasmic tail. In some cases, each chain of the TCR possesses one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR can contain short connecting sequences in which a cysteine residue forms a disulfide bond, thereby linking the two chains of the TCR. In some embodiments, a TCR has an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region can anchor the protein in the cell membrane and associate with invariant subunits of the CD3 signaling apparatus or complex. The intracellular tails of CD3 signaling subunits (e.g., CD3y, CD35, CD3s and CD3ζ chains) contain one or more immunoreceptor tyrosine-based activation motif or ITAM that are involved in the signaling capacity of the TCR complex.

In some embodiments, the TCR is a heterodimer of two chains α and β (or optionally γ and δ) or it is a single chain TCR construct. In some embodiments, the TCR is a heterodimer containing two separate chains (α and β chains or γ and δ chains) that are linked, such as by a disulfide bond or disulfide bonds.

In some embodiments, the TCR is generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR are obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g., cytotoxic T cell), T-cell hybridomas or other publicly available source. In some embodiments, the T-cells are obtained from in vivo isolated cells. In some embodiments, the T-cells are obtained from a biopsy tumor sample. In some embodiments, the TCR is a thymically selected TCR. In some embodiments, the TCR is a neoepitope-restricted TCR. In some embodiments, the T-cells are a cultured T-cell hybridoma or clone. In some embodiments, the TCR or antigen-binding portion thereof is synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells are amplified from tumor-infiltrating lymphocytes (TILs). In some embodiments, TCR libraries are generated from CD4+ or CD8+ cells. In some embodiments, the TCRs are amplified from a T cell source of a normal of healthy subject, e.g., normal TCR libraries. In some embodiments, the TCRs are amplified from a T cell source of a diseased subject, e.g., diseased TCR libraries. In some embodiments, degenerate primers are used to amplify the gene repertoire of Vα and Vβ, such as by RT-PCR in samples, such as T cells, obtained from humans. In some embodiments, scFv libraries are assembled from naive Vα and V3 libraries in which the amplified products are cloned or assembled to be separated by a linker. Depending on the source of the subject and cells, the libraries can be HLA allele-specific. Alternatively, in some embodiments, TCR libraries are generated by mutagenesis or diversification of a parent or scaffold TCR molecule. In some aspects, the TCRs are subjected to directed evolution, such as by mutagenesis, e.g., of the α or β chain. In some cases, particular residues within CDRs of the TCR are altered. In some embodiments, selected TCRs are modified by affinity maturation. Antigen-specific T cells can be selected, such as by screening to assess CTL activity against the peptide. In some aspects, TCRs, e.g., present on the antigen-specific T cells, can be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al., (2003) Nat Immunol, 4, 55-62; Holler et al., (2000) Proc Natl Acad Sci USA, 97, 5387-92), phage display (Li et al., (2005) Nat Biotechnol, 23, 349-54), or T cell display (Chervin et al, (2008) Immunol Methods, 339, 175-84). In some embodiments, display approaches involve engineering, or modifying, a known parent or reference TCR. For example, a wild-type TCR can be used as a template for producing mutagenized TCRs in which one or more residues of the CDRs are mutated, and mutants with an desired altered property, such as higher affinity for a desired target antigen, are selected.

In some embodiments, peptides of a target polypeptide for use in producing or generating a TCR of interest are known or are readily identified by a skilled artisan. In some embodiments, peptides suitable for use in generating TCRs or antigen-binding portions are determined based on the presence of an HLA-restricted motif in a target polypeptide of interest. In some embodiments, peptides are identified using computer prediction models known to those of skill in the art.

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, or WO2011/044186. In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR a chain variable region sequence is fused to the N terminus of a sequence corresponding to a TCR a chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR a chain containing a variable a domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR a chain and TCR β chain together.

In some embodiments, the TCR is a scTCR. A scTCR can be generated using methods known to those of skill in the art, see e.g., Soo Hoo, W. F. et ah, PNAS (USA) 89, 4759 (1992); Wiilfing, C. and Pliickthun, A., J. Mol. Biol. 242, 655 (1994); Kurucz, I. et al, PNAS (USA) 90 3830 (1993); International published PCT Nos. WO 96/13593, WO 96/18105, WO99/60120, WO99/18129, WO 03/020763, WO2011/044186; and Schlueter, C. J. et al., J. Mol. Biol. 256, 859 (1996). In some embodiments, a scTCR contains an introduced non-native disulfide interchain bond to facilitate the association of the TCR chains (see e.g., International published PCT No. WO 03/020763). In some embodiments, a scTCR is a non-disulfide linked truncated TCR in which heterologous leucine zippers fused to the C-termini thereof facilitate chain association (see e.g., International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR a chain variable region, a second segment constituted by an amino acid sequence corresponding to a TCR 3 chain variable region sequence fused to the N terminus of an amino acid sequence corresponding to a TCR 3 chain constant domain extracellular sequence, and a linker sequence linking the C terminus of the first segment to the N terminus of the second segment.

In some embodiments, a scTCR contains a first segment constituted by an a chain variable region sequence fused to the N terminus of an a chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an a chain variable region sequence fused to the N terminus of a sequence a chain extracellular constant and transmembrane sequence, and, optionally, a linker sequence linking the C terminus of the first segment to the N terminus of the second segment.

In some embodiments, the linker of a scTCRs that links the first and second TCR segments is any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker contains from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine. In some embodiments, the linker has the sequence GSADDAKKDAAKKDGKS. In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable. In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond is formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, the engineered cell therapy involve multi-targeting strategies, such as expression of two or more genetically engineered receptors on the cell, e.g., T cell, each recognizing the same of a different antigen and typically each including a different intracellular signaling component. Such multi-targeting strategies are described, for example, in PCT Pub. No. WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells, e.g., T cells, include a receptor expressing a first genetically engineered antigen receptor (e.g., CAR or TCR) which is capable of inducing an activating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, T cells are engineered to become T cells redirected for antigen-unrestricted cytokine-initiated killing (TRUCKs), which encode genes for cytokine production to augment CAR-T activity or suicide genes to prevent toxicity, such as those described in Chmielewski M et al. Expert Opin Biol Ther. 2015; 15(8):1145-54, which is incorporated herein by reference in its entirety.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

In some embodiments, engineered TCRs, e.g., for treating cancer using the methods as described herein, include those having immune cell activation function in response to a cancer associated antigen. Non-limiting examples include antigen-specific TCRs, Monoclonal TCRs (MTCRs), Single chain MTCRs, High Affinity CDR2 Mutant TCRs, CDI-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1): 145-64; Schmitt et al. Blood. 2013; 122(3):348-56; Chapuls et al. Sci Transl Med. 2013; 5(174):174ra27; Thaxton et al. Hum Vaccin Immunother. 2014; 10(11): 3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Marr et al. Clin Exp Immunol. 2012; 167(2):216-25 (PMID:22235997); Zhang et al. Adv Drug Deliv Rev. 2012; 64(8): 756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. I Drugs. 2006; 9(8): 554-9; Li et al. Nat Biotechnol. 2005; 23(3):349-54; Dunn et al. Protein Sci. 2006; 15(4):710-21; Liddy et al. Mol Biotechnol. 2010; 45(2); Liddy et al. Nat Med. 2012; 18(6): 980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; the disclosures of which are incorporated herein by reference in their entirety. In some instances, useful TCRs include those targeting one of the following antigens: NY-ESO-1, MART-1, MAGE-A3, MAGE-A3, CEA, gp100, WT1, HBV, gag (WT and/or a/6), P53, TRAIL bound to DR4, HPV-16 (E6 and/or E7), Survivin, KRAS mutants, SSX2, MAGE-A10, MAGE-A4, AFP, and the like.

### Cells and Cell Engineering in Engineered Cell Therapy

Among the cells expressing the receptors and administered in an engineered cell therapy as described herein are engineered cells. The cell engineering can involve introduction of a nucleic acid encoding a recombinant or engineered component into a composition containing the cells, such as by retroviral transduction, transfection, or transformation.

The cells can be eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including dendritic cells (DCs), monocytes, macrophages, and lymphocytes (e.g., T cells, B cells, or natural killer (NK) cells). Other exemplary cells include stem cells, such as multipotent and pluripotent stem cells, including induced pluripotent stem cells (iPSCs). The cells can be primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells can be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and reintroducing them into the same subject, before or after cryopreservation. Among the sub-types and subpopulations of T cells and/or of CD4+ and/or of CD8+ T cells are naive T (TN) cells, effector T cells (TEFF), memory T cells and sub-types thereof, such as stem cell memory T (TSCMX central memory T (TCMX effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells comprise natural killer (NK) cells. In some embodiments, the cells comprise monocytes or granulocytes, e.g., myeloid cells, macrophages, neutrophils, dendritic cells, mast cells, eosinophils, and/or basophils.

In some embodiments, the nucleic acids are heterologous, e.g., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the engineered cell therapy for which cells are being isolated, processed, and/or engineered.

Accordingly, the cells in some embodiments are primary cells, e.g., primary human cells. The samples can include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Non-limiting exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of engineered cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al, Blood. 1:72-82 (2012); Wang et al, J Immunother. 35(9):689-701 (2012). In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al, Lab Chip 10, 1567-1573 (2010); and Godin et al., J Biophoton. 1(5):355 376 (2008). In both cases, cells can be labeled with multiple markers, allowing for the isolation of well-defined T cell subsets at high purity.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation can further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some embodiments, the engineered cell therapy provided herein comprises tumor infiltrating lymphocyte (TIL) therapy. Therapeutic agent of TIL therapy can comprise tumor infiltrating lymphocytes. The tumor filtrating lymphocytes can refer to lymphocytes (e.g., white blood cells) that have left the bloodstream and migrated towards a tumor. The tumor infiltrating lymphocytes that can be used in an engineered cell therapy can include both mononuclear and polymorphonuclear immune cells, e.g., T cells, B cells, natural killer cells, macrophages, neutrophils, dendritic cells, mast cells, eosinophils, and basophils. TILs can be found in the tumor stroma and within the tumor itself. For therapeutic use, TILs can be obtained from a tumor of a subject, e.g., isolated from a resected tumor tissue. In some embodiments, TILs are expanded ex vivo from surgically resected tumors. For isolation, the resected tumor tissue can be fragmented or dissociated into single cell suspensions, from which TILs can be isolated via well-known separation techniques. Multiple individual cultures can be established, grown separately and assayed for specific tumor recognition. TILs can be expanded over the course of a few weeks with a high dose of IL-2 in 24-well plates, similar to the cell expansion as described above. TIL lines can then be subject to selection for their presentation of tumor reactivity, and the selected TIL lines can then be further expanded. In some embodiments, the selected TILs are further expanded in a rapid expansion protocol (REP), which uses anti-CD3 activation for a period of about two weeks. The final post-REP TIL can then infused back into the tumor patient.

### Administration of an Engineered Cell Therapy

In some embodiments, the engineered cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the engineered cell therapy is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means. The cells are administered in a dosing regimen to achieve a therapeutic effect, such as a reduction in tumor burden. Dosing and administration can depend in part on the schedule of administration of the agonist of the immune checkpoint protein, which can be administered prior to, subsequent to and/or simultaneously with initiation of administration of the engineered cell therapy. Various dosing schedules of the engineered cell therapy include but are not limited to single or multiple administrations over various time-points, bolus administration, and pulse infusion.

In some embodiments, the cells of the engineered cell therapy, such as T cells engineered with a recombinant antigen receptor, e.g., CAR or TCR, or tumor-infiltrating cells, is provided as a composition or formulation, such as a pharmaceutical composition or formulation. Such compositions can be used in accord with the provided methods, such as in the treatment of diseases, conditions, and disorders.

In some embodiments, the engineered cell therapy, such as engineered T cells (e.g., CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives can include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous suspension solutions. The formulation or composition can also contain more than one active ingredient useful for the particular indication, disease, or condition being treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells can be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it can be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The formulations to be used for in vivo administration are generally sterile. Sterility may be readily accomplished, e.g., by filtration through sterile filtration membranes.

For the treatment of disease, the appropriate dosage can depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

In some cases, the cell therapy is administered as a single pharmaceutical composition comprising the cells. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

In some embodiments, a dose of cells is administered to subjects in accordance with the provided methods. In some embodiments, the size or timing of the doses is determined as a function of the particular disease or condition in the subject.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the engineered cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg. In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g., antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g., antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages of the cells can vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject. In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, where the subject is a human, the dose includes between about 1×10⁶ and 3×10⁸ total recombinant receptor (e.g., CAR)-expressing cells, e.g., in the range of about 1×10⁷ to 2×10⁸ such cells, such as 1×10⁷, 5×10⁷, 1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁵ to 5×10⁸ total recombinant receptor-expressing T cells or total T cells, 1×10⁵ to 1×10⁸ total recombinant receptor-expressing T cells or total T cells, from or from about 5×10⁵ to 1×10⁷ total recombinant receptor-expressing T cells or total T cells, or from or from about 1×10⁶ to 1×10⁷ total recombinant receptor-expressing T cells or total T cells, each inclusive.

In some embodiments, the T cells of the dose include CD4+ T cells, CD8+ T cells or CD4+ and CD8+ T cells. In some embodiments, for example, where the subject is human, the CD8+ T cells of the dose, including in a dose including CD4+ and CD8+ T cells, includes between about 1×10⁶ and 1×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷ or 1×10⁸ total such cells, or the range between any two of the foregoing values. In some embodiments, the patient is administered multiple doses, and each of the doses or the total dose can be within any of the foregoing values. In some embodiments, the dose of cells comprises the administration of from about 1×10⁷ to 7.5×10⁷ total recombinant receptor-expressing CD8+T cells, 1×10⁷ to 2.5×10⁷ total recombinant receptor-expressing CD8+ T cells, from or from about 1×10⁷ to 7.5×10⁷ total recombinant receptor-expressing CD8+ T cells, each inclusive. In some embodiments, the dose of cells comprises the administration of or about 1×10⁷, 2.5×10⁷, 5×10⁷, 7.5×10⁷, or 1×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of engineered cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, such as no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days. Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days. In some embodiments, the dose of cells can be large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio. In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In some embodiments, the cells are administered at or within a tolerated range of a desired output ratio of multiple cell populations or sub-types, such as CD4+ and CD8+ cells or sub-types. In some aspects, the desired ratio is a specific ratio or is a range of ratios, for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, one or more subsequent dose of cells is administered to the subject. In some embodiments, the subsequent dose of cells is administered greater than or greater than about 7 days, 14 days, 21 days, 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

### Methods of Use

Systems and compositions of the present disclosure are useful for a variety of applications. Diseases and disorders that can be treated using engineered cells of the present disclosure include inflammatory conditions, cancer, and infectious diseases. In some embodiments, systems of compositions provided herein are used to treat cancer.

Non-limiting examples of cancers that can be treated by the methods, compositions, and regimens of the present disclosure include acute lymphoblastic leukemia (ALL) (including non T cell ALL), acute myeloid leukemia, B cell prolymuphocytic leukemia, B cell acute lyrnphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, or a combination thereof.

A tumor treated with the methods, compositions, and regimens herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g., in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment. Death of the tumor cells can be determined by any suitable method, including, but not limited to, counting cells before and after treatment, or measuring the level of a marker associated with live or dead cells (e.g., live or dead target cells). Degree of cell death can be determined by any suitable method. In some embodiments, degree of cell death is determined with respect to a starting condition. For example, an individual can have a known starting amount of target cells, such as a starting cell mass of known size or circulating target cells at a known concentration. In such cases, degree of cell death can be expressed as a ratio of surviving cells after treatment to the starting cell population. In some embodiments, degree of cell death can be determined by a suitable cell death assay. A variety of cell death assays are available, and can utilize a variety of detection methodologies. Examples of detection methodologies include, without limitation, the use of cell staining, microscopy, flow cytometry, cell sorting, and combinations of these.

When a tumor is subject to surgical resection following completion of a therapeutic period, the efficacy of treatment in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%). In some embodiments, the necrosis percentage of the resected tissue is 100%, that is, no living tumor tissue is present or detectable.

### Examples

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Cloning and Purification of Targeted Cytokine Constructs

Recombinant DNA Techniques

Techniques involving recombinant DNA manipulation were previously described in Sambrook et al., Molecular cloning: A laboratory manual; Cold Spring Harbor Laboratory Press, Cold Spring Harbor, N.Y., 1989. All reagents were used according to the manufacturer's instructions. DNA sequences were determined by double strand sequencing.

Gene Synthesis

Desired gene segments were either generated by PCR using appropriate templates or synthesized at Thermo Scientific (Pleasanton, CA), ATUM (Newark, CA), Genewiz (South Plainfield, NJ), or GeneScript (Piscataway, NJ) from synthetic oligonucleotides. The gene segments flanked by designed restriction endonuclease cleavage sites were digested out and later cloned into their respective expression vectors. DNA was purified from transformed bacteria and concentration determined by UV visible spectroscopy. DNA sequencing was used to confirm the DNA sequences of the subcloned gene fragments.

Isolation of Antibody Genes

Antibodies targeting a domain or a receptor expressed by an engineered cell (e.g., antibodies targeting an scFv expressed by the engineered cell, or a tag expressed by the engineered cell, or a tag that is part of a CAR or TCR expressed by the engineered cell) were generated using either in vitro display system or in vivo immunizations. For in vitro display method, a non-immune human antibody phage library was panned for 5 to 6 rounds to isolate antibodies against the target antigen. After the panning, individual phage clones that exhibited specific binding to target antigen over non-specific antigens in ELISA were identified. DNA fragments of heavy and light chain V-domain of the specific binders were subsequently cloned and sequenced. Meanwhile, antibodies were also generated from immunizing mice and llamas with the recombinant form of the antigens. From the mouse immunization, hybridoma method was used to isolate the antibody. Briefly, after immunization, B cells from spleen and/or lymph nodes were fused with a myeloma cell line to generate the hybridoma cells. Hybridoma clones were then individually screened using ELISA to identify the clones expressing antibodies specific for the antigen. Finally, DNA fragments of heavy and light chain V-domain of the antibody were cloned from the specific hybridoma and later sequenced. For the llama immunization, antibody genes were cloned from peripheral B cells and ligated into the phagemid vector to generate a phage display antibody library. Antibodies were then isolated through panning the phage library against the antigens of interest. After the panning, individual phage clones that exhibited specific binding to target antigen over non-specific antigens were identified using ELISA. DNA fragments of heavy and light chain V-domain of the specific binders were then subsequently cloned and sequenced. Antibodies from non-human origins (mouse and llama) were then humanized to remove non-human framework and complementarity-determining region mutations.

Cloning of Targeted Cytokine Constructs

General information regarding the nucleotide sequences of human immunoglobulins light and heavy chains is given in: IMGT® (the international ImMunoGeneTics information System®) from Lefranc et al. IMGT®, the international ImMunoGeneTics information System® 25 years on. Nucleic Acids Res. 2015 January; 43. The amplified DNA fragments of heavy and light chain V-domains were inserted in frame into the human IgG1 containing mammalian expression vector. For exemplary targeted cytokine constructs containing an IL-2 cytokine, or a functional fragment, or a variant thereof, the IL-2 portions of the constructs were cloned in frame with the heavy chain using a (G4S)3 15-mer linker between the C-terminus of the IgG heavy chain and the N-terminus of IL-2. The C-terminal lysine residue of the IgG heavy chain was eliminated after fusing the IL-2 portion. To generate the construct in which a single IL-2 gene was fused to a full IgG, two heavy chain plasmids needed to be constructed and transfected for heterodimerization facilitated by a knob-into-hole modification in the IgG CH3 domains. The “hole” heavy chain connected to the IL-2 portion carried the Y349C, T366S, L368A and Y407V mutations in the CH3 domain, whereas the unfused “knob” heavy chain carried the S354C and T366W mutations in the CH3 domain (EU numbering). To abolish FcγR binding/effector function and prevent FcR co-activation, the following mutations were introduced into the CH2 domain of each of the IgG heavy chains: L234A/L235A/G237A (EU numbering). The expression of the antibody-IL-2 fusion constructs was driven by an CMV promoter and transcription terminated by a synthetic polyA signal sequence located downstream of the coding sequence.

Preparation of Exemplary Targeted Cytokine Constructs with IL-2 Polypeptides

Constructs encoding targeted cytokine constructs with IL-2 polypeptides as used in the examples were produced by co-transfecting exponentially growing Expi293 cells with the mammalian expression vectors using polyethylenimine (PEI). Briefly, IL-2 containing targeted cytokine constructs were first purified by affinity chromatography using a protein A matrix. The protein A column was equilibrated and washed in phosphate-buffered saline (PBS). The targeted cytokine constructs were eluted with 20 mM sodium citrate, 50 mM sodium chloride, pH 3.6. The eluted fractions were pooled and dialyzed into 10 mM MES, 25 mM sodium chloride pH 6. The proteins were further purified using ion-exchange chromatograph (Mono-S, GE Healthcare) to purify the heterodimers over the homodimers. After loading the protein, the column is washed with 10 mM MES 25 mM sodium chloride pH 6. The protein was then eluted with increasing gradient of sodium chloride from 25 mM up to 500 mM in 10 mM MES pH 6 buffer. The major eluent peak corresponding to the heterodimer was collected and concentrated. The purified protein was then polished by size exclusion chromatography (Superdex 200, GE Healthcare) in PBS.

The protein concentration of purified IL-2 containing targeted cytokine constructs was determined by measuring the optical density (OD) at 280 nm, using the molar extinction coefficient calculated on the basis of the amino acid sequence. Purity, integrity and monomeric state of the targeted cytokine constructs were analyzed by SDS-PAGE in the presence and absence of a reducing agent (5 mM 1,4-dithiothreitol) and stained with Coomassie blue (SimpleBlue™ SafeStain, Invitrogen). The NuPAGE® Pre-Cast gel system (Invitrogen) was used according to the manufacturer's instructions (4-20% Tris-glycine gels or 3-12% Bis-Tris). The aggregate content of immunoconjugate samples was analyzed using a Superdex 200 10/300 GL analytical size-exclusion column (GE Healthcare).

Cloning of Lentiviral Vector Constructs

Lentiviral vectors (LVV) used in this study were modified from the plasmid pALD-Lenti GFP (Aldevron). Specifically, the promoter region of pALD-Lenti GFP was replaced with an EF1a promoter sequence. CAR gene expressing FMC63 scFv was cloned in frame to generate pLVV-FMC63 plasmid. Also, the myc polypeptide (EQKLISEEDL) was inserted into either N- or C-terminal region of FMC63 to create pLVV-FMC63-N-myc or pLVV-FMC63-C-myc, respectively. To co-express EGFRt, a sequencing consisting of P2A (ATNFSLLKQAGDVEENPGP) and EGFRt transgenes was inserted downstream of FMC63 scFv region to generate pLVV-FMC63-EGFRt. For TCR construct, a 5′ to 3′ sequence consisting of N-myc-TCR alpha chain-P2A-HA tag-TCR beta chain was cloned in frame to generate the pLVV-TCR.

The sequence of the EGFTt tag is as follows:

RKVCNGIGIGEFKDSLSINATNIKHFKNCTSISGDLHILPVAFRGDSFT
HTPPLDPQELDILKTVKEITGFLLIQAWPENRTDLHAFENLEIIRGRTK
QHGQFSLAVVSLNITSLGLRSLKEISDGDVIISGNKNLCYANTINWKKL
FGTSGQKTKIISNRGENSCKATGQVCHALCSPEGCWGPEPRDCVSCRNV
SRGRECVDKCNLLEGEPREFVENSECIQCHPECLPQAMNITCTGRGPDN
CIQCAHYIDGPHCVKTCPAGVMGENNTLVWKYADAGHVCHLCHPNCTYG
CTGPGLEGCPTNGPKIPSIATGMVGALLLLLVVALGIGLFM

Production of Lentivirus Particles

Lentivirus were generated using the 3-plasmid packaging system. Specifically, three plasmids, pALD-VSV-G, pALD-GagPol, and pALD-Rev (Aldevron), were cotransfected with the pLVV vector into 239 cells to generate the virus. Lentivirus is produced using the Gibco™ LV-MAX™ Lentiviral Production System (Catalog Number A35684), following the manufacturer's protocol. Briefly, the 293 cells were seeded as 0.5 million cells per m1, and cultured in a 37° C. incubator with a humidified atmosphere of 8% C02 and shaking (125 rpm) for 3 days. After the cell density reached 5-6 million cells per mL, culture was then diluted to 3.5 million cells per mL for overnight culture. Next day, cells were transfected with the plasmids following manufacturer's protocol. Culture was harvested 48-55 hours post-transfection. Cells were then spun down at 1300×g for 15 min. Culture supernatant containing the lentivirus was then collected and filtered, and later stored at −80° C.

### Example 2: In Vitro Assays to Demonstrate Preferential Activation and Proliferation of CAR-Engineered Over Non-Engineered T Cells

Generation and Culture of CAR T Cells

T cells were isolated using the RosetteSep™ Human T Cell Enrichment Cocktail (Stem cell) and resuspended at 1×10⁶/mL in AIM-V media (Gibco) and manufacturer recommended concentration of T Cell TransAct™, human anti-CD3/anti-CD28 (Miltenyi) supplemented with either 5 ng/mL rhIL-2 (R&D) or 1 ng/mlL of both rhIL-7 and rhIL-15 (Peprotech) for 36-48 hours. Cells were then washed 1× with and resuspended in Opti-MEM (Gibco) and plated in 25 μl at 50×10³cells/well in 96 well flat bottom plates. T cells were transduced by spinoculation at 1000 g, 30° C. for 90 minutes with premade FMC63-41BB-3z lentiviral particles diluted 1/8 (ProMab) in the presence of 5 μg/ml protamine sulfate (MP biomedicals). Six to eight hours after spinoculation, 200 μl of AIM-V media (Gibco) supplemented with 5 ng/ml rhIL-2 (R&D) or 1 ng/ml rhIL-7 and rhIL-15 (Peprotech) was added. Forty-eight hours after media addition, cells were checked for CAR expression by flow cytometry using anti-FMC63 (FM63, Acro bio). Cells were supplemented with AIM-V or RPMI media (+ FBS and Glutamax) containing respective cytokines every other day, and cells were kept between 0.2-2×10⁶/mL densities for up to 28 days.

STAT5 Activation Assay for Measuring Selective Activation of CAR-Engineered Over Non-Engineered T Cells

Typically, CAR T cells were washed 3× and resuspended in serum-free RPMI1640 or AIMN-V media at 2×10⁶ cells/mL and aliquoted into 96-well U-bottom plates (50 μL per well) and rested overnight (18-24 hrs). IL-2 containing targeted cytokine constructs and control proteins, such as recombinant human IL-2 and control (HA-targeted) fusion proteins, were diluted to desired concentrations and added to wells (50 μL added as 2× stimulus). Incubation was typically performed for 30 min at 37° C. Cells were then stained with antibodies against surface markers: CD8α (SK1, Biolegend; RPA-T8, Biolegend), and FMC63scFv (FM63, Acrobio) and fixed with 4% PFA at room temperature for 10 min. After fixation, cells were permeabilized in pre-chilled Phosflow Perm buffer III (BD Biosciences) according to manufacturer's protocol. After permeabilization, cells were stained with antibodies against intracellular pSTAT5 [pY694], clone 47, BD Biosciences, and surface markers CD3 (UCHT1, BD Biosciences), CD4 (RPA-T4, Biolegend), and CD25 (M-A251, Biolegend) and analyzed on a flow cytometer. CAR+ cells were determined by FMC63 scFv binding. Data were expressed as percent pSTAT5 positive, and in some cases as pSTAT5 mean fluorescence intensity (MFI), and imported into GraphPad Prism to determine EC₅₀ values for each construct.

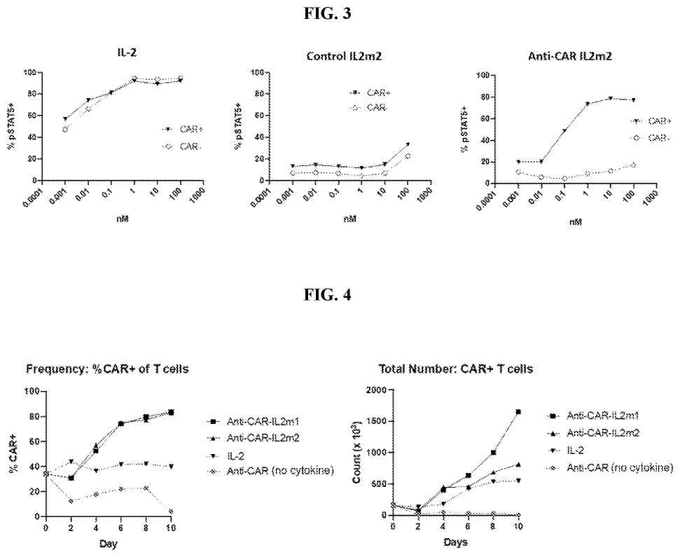

The results are shown in FIG. 2 for constructs comprising anti-CAR antibody and cytokine IL2m1 and in FIG. 3 for construct comprising anti-CAR antibody and cytokine IL2m2.

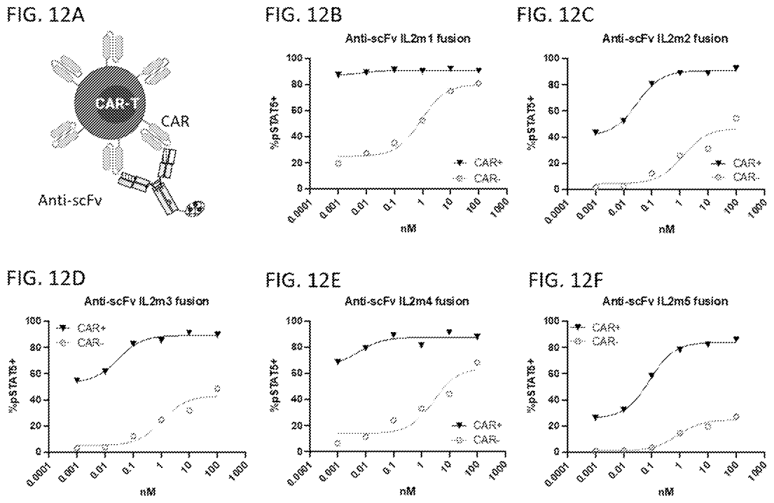

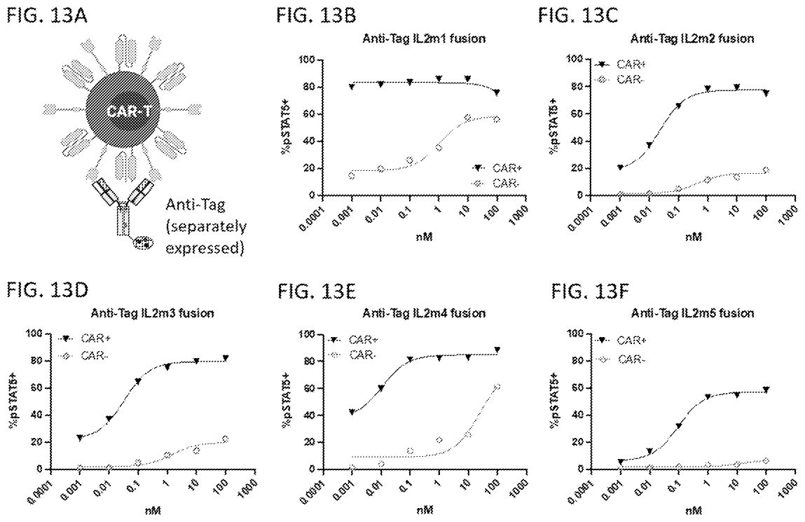

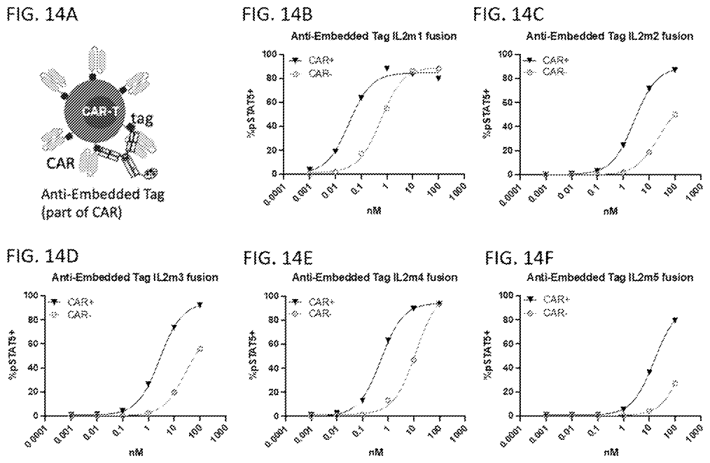

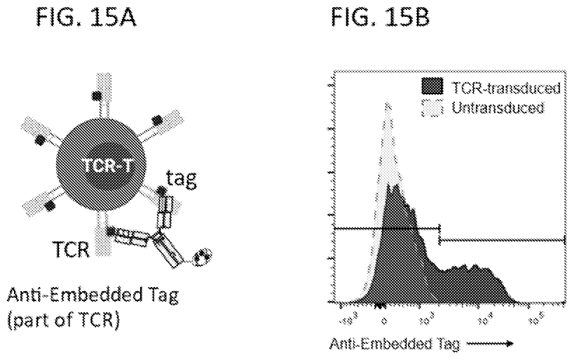

The STAT5 activation assay was carried out with additional constructs shown in FIGS. 12A, 13A, 14A, and 15A. The construct shown in FIG. 13A comprises an anti-scFv antibody (e.g., an anti-idiotype antibody for a CAR which comprises a CD19 targeting scFv (FMC63scFv)) and an IL-2 cytokine variant (IL2m1, ILm2, IL2m3, IL2m4, or ILm5); the construct shown in FIG. 14A comprises an anti-tag antibody targeting a tag separately expressed on the CAR T cell surface (e.g., the tag is EGFRt and the antibody is an anti-EGFRt) and an IL-2 cytokine variant (IL2m1, ILm2, IL2m3, IL2m4, or ILm5); the construct shown in FIG. 15A comprises an anti-tag antibody targeting an embedded tag that is expressed within the CAR molecule (e.g., the tag is myc and the antibody is an anti-myc antibody) and an IL-2 cytokine variant (IL2 m1, ILm2, IL2m3, IL2m4, or ILm5); the construct shown in FIG. 16A comprises an anti-tag antibody targeting an embedded tag that is expressed within the TCR (e.g., the tag is myc and the antibody is an anti-myc antibody) and a cytokine (IL2m1, ILm2, IL2m3, IL2m4, or ILm5).

The results are shown in FIGS. 12B-12F (for the construct of FIG. 12A); FIGS. 13B-13F (for the construct of FIG. 13A); FIGS. 14B-14F (for the construct of FIG. 14A); and FIG. 15B (for the construct of FIG. 15A). For each construct, STAT5 activation was significantly enhanced for the CAR+ cells compared to the STAT5 activation for the CAR− cells, indicating selective activation of the CAR-engineered T cells over non-engineered T cells with several exemplary targeted constructs of this disclosure.

The results are shown in FIG. 15B (for the cell binding domain of the construct of FIG. 15A). The cell binding domain of the depicted construct binds an embedded tag that is expressed within a TCR.

Selective Outgrowth of CAR-Engineered T Cells Over Non-Engineered T Cells with Targeted Cytokine Treatment In Vitro

CAR T cells, generated and maintained as above, were washed 3× with RPMI1640 (Gibco)+10% FBS (Gibco) and 1% Glutamax (Gibco) and resuspended at 0.5×10⁶/mL and seeded at 0.5 mL per 48 well plate well in the respective CAR-directed cytokine concentrations. Frequency and numbers of CAR+ cells were measured every other day, where 100 μL of cells would be removed for surface and intracellular flow cytometry, and 100 μL of 5× concentration of CAR-targeted constructs supplemented. In the case where proliferation increased density to greater than 2×10⁶ cells/m1, wells were split into 2. Cells were stained for the following markers CD3 (UCHT1, BD Biosciences), CD4 (RPA-T4, Biolegend), CD8α (SK1, Biolegend; RPA-T8, Biolegend), FMC63 scFv (FM63, Acrobio), CD62L (DREG-56, Biolegend), CD45RO (UCHL1, Biolegend), CD45RA (HI100, Biolegend) CCR7(150503, BD), and TCF1 (7F11A10, Biolegend) and analyzed on a flow cytometer. Total numbers were back calculated from the number of events collected by the flow cytometer over a fixed volume. The results shown in FIG. 4 demonstrate the selective outgrowth of CAR-engineered T cells (measured by % CAR+ cells (left panel) or total number of CAR+ cells (right panel) in the presence of exemplary CAR-directed constructs of this disclosure (anti-CAR-IL2 m1 and anti-CARIL2m2), as compared to control IL2 or anti-CAR antibody that is not fused to a cytokine.

### Example 3: In Vivo Mouse Models to Demonstrate Better Engraftment, Persistence, and Characteristics of CAR-Engineered T Cells with Targeted Cytokine Treatment

For the subcutaneous NSG model, NSG mice are injected with 1×10⁶ NALM6 or Raji tumors subcutaneously on the flank followed by infusion of 0.1-0.5×10⁶ CAR-T cells, which are produced as described in Example 2, when tumors reach 5-8 mm in diameter. Mice are then treated with PBS or CAR-targeted IL-2 at the indicated time points after CAR-T infusion. Tumor size is measured via calipers at least twice weekly, and peripheral blood is taken and analyzed by flow cytometry at the indicated time points to characterize CAR-T frequency and phenotype. Mice are sacrificed when tumor burden exceeded 1000 mm³.

For the i.v. NSG model, NSG mice are injected with 0.5×10⁶ luciferase transduced NALM6 or Raji tumors intravenously followed by infusion of 0.5M CAR-T cells, produced as described in Example 2. Mice are then treated with PBS or CAR-targeted IL-2 at the indicated time points after CAR-T infusion. Peripheral blood is taken and analyzed by flow cytometry at the indicated time points in order to characterize CAR-T frequency and phenotype. Mice are imaged using In Vivo Imaging System (IVIS) at least twice weekly to measure tumor burden via luminescence.

For the s.c. syngeneic models, C57BL6/J mice are injected subcutaneously with 1×10⁶ hCD19-transduced B16F10, MC38, or EL4 tumors. After tumors reach 5-8 mm in diameter, 1×10⁶ hCD19-CAR-T transduced mouse T cells are infused intravenously. Mice are treated with either CAR-targeted IL-2 or PBS as shown following CAR-T infusion. Peripheral blood is taken and analyzed by flow cytometry at the indicated time points to characterize CAR-T frequency and phenotype. In some instances, mice are preconditioned prior to transfer using cyclophosphamide. For TIL analysis, tumors are taken after treatment, disaggregated, and analyzed by flow cytometry for characterization of functionality and phenotype. For these experiments, the mouse hCD19-CAR-T cells are produced from CD3+ T cells selected from C57BL6/J splenocytes activated in vitro using CD3/CD28 beads and transduced using retrovirus encoding hCD19-CAR-T constructs with 41BB/CD3z or CD28/CD3z intracellular domains.

### Example 4 Evaluation of STAT3 Activation in Engineered Cells

The ability of IL-10 or IL-21 to activate engineered cells was determined by measuring the phosphorylation of STAT3 by flow cytometry. Engineered cells were resuspended in serum-free RPMI1640 media at 40×10⁶ cells/mL and aliquoted into 96-well U-bottom plates (50 μL per well). Targeted fusion proteins and control proteins including recombinant wildtype cytokine and control fusion proteins, were diluted to desired concentrations and added to wells (50 μL added as 2× stimulus). Incubation was typically performed for 10-20 min at 37° C. Antibodies that could not be applied following methanol permeabilization—CD8a (SK1, Biolegend), CD4 (RPA-T4, Biolegend), CD62L (DREG-56, Biolegend), and CD19 (HIB19, Biolegend)—were added directly to the wells immediately following stimulation and incubated on ice for 10 min. Staining was stopped with 100 μL ice cold 8% PFA (4% final) for 10 min on ice. Cells were washed with wash buffer (2% FBS in PBS). Cells were permeabilized in 100 μL pre-chilled Phosflow Perm buffer III (BD Biosciences) for 45 minutes on ice. Cells were washed wash buffer and stained for 30-45 min at 4° C. with antibodies that bind to engineered cells over non-engineered cells. Data were expressed as percent pSTAT3 positive, and in some cases as pSTAT3 mean fluorescence intensity (MFI), and imported into GraphPad Prism.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A targeted cytokine construct with an engineered cell comprising:
a cell binding domain that targets at least one of: (i) a domain of a chimeric antigen receptor (CAR) or a T cell receptor (TCR) exogenously introduced into the engineered cell; (ii) a tag molecule selectively expressed on the surface of the engineered cell; (iii) a polypeptide tag that is part of a CAR exogenously introduced into the engineered cell; (iv) a polypeptide tag that is part of a TCR exogenously introduced into the engineered cell, or (vi) any combination of (i)-(v), and
a cytokine protein or a functional fragment or a variant thereof.

2. The targeted cytokine construct of claim 1, wherein the targeted cytokine construct selectively activates the engineered cell with 10-fold or greater potency as compared to activation of a non-engineered cell.

3. The targeted cytokine construct of claim 2, wherein the potency is measured by a pSTAT5 or a pSTAT3 activation assay.

4. The targeted cytokine construct of any one of claims 1-3, wherein the domain of the CAR is an scFv.

5. The targeted cytokine construct of any one of claims 2-4, wherein the non-engineered cell does not express on its surface: the CAR, the TCR, or the tag molecule.

6. The targeted cytokine construct of any one of claims 1-5, wherein the cell binding domain comprises an antibody or an antigen binding fragment thereof.

7. The targeted cytokine construct of claim 6, wherein the antibody or an antigen binding fragment thereof is bivalent or monovalent.

8. The targeted cytokine construct of any one of claims 1-7, wherein the cytokine is selected from the group consisting of: IL-2, IL-7, IL-10, IL-15, and IL-21, or a functional fragment thereof, or a variant thereof, or any combinations thereof.

9. The targeted cytokine construct of 8, wherein the cytokine is the IL-2 polypeptide, or a functional fragment thereof, or a variant thereof.

10. The targeted cytokine construct of claim 9, wherein the IL-2 polypeptide exhibits reduced binding affinity by at least about 50% to an IL-2Rα polypeptide having an amino acid sequence of SEQ ID NO:2, compared to the binding affinity of the wild-type IL-2 polypeptide with an amino acid sequence of SEQ ID NO:1

11. The targeted cytokine construct of claim 10, wherein the IL-2 polypeptide exhibits reduced binding affinity by at least about 50% to an IL-2Rβ polypeptide having an amino acid sequence of SEQ ID NO:3, and/or reduced binding affinity by at least about 50% to an IL-2Rγ polypeptide having an amino acid sequence of SEQ ID NO:4 compared to the binding affinity of the wild-type IL-2 polypeptide with an amino acid sequence of SEQ ID NO:1.

12. The targeted cytokine construct of any one of claims 9-11, wherein the IL-2 polypeptide comprises the sequence of SEQ ID NO:1 with one or more amino acid substitutions relative to SEQ ID NO:1, and wherein the one or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130.

13. The targeted cytokine construct of claim 12, wherein the one or more substitution(s) comprise an F42A or F42K amino acid substitution relative to SEQ ID NO:1.

14. The targeted cytokine construct of claim 13, wherein the one or more substitution(s) further comprise an R38A, R38D, R38E, E62Q, E68A, E68Q, E68K, or E68R amino acid substitution relative to SEQ ID NO:1.

15. The targeted cytokine construct of claim 14, wherein the one or more substitution(s) further comprise an H16E, H16D, D20N, M23A, M23R, M23K, S87K, S87A, D84L, D84N, D84V, D84H, D84Y, D84R, D84K, N88A, N88G, N88S, N88T, N88R, N88I, N88D, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1.

16. The targeted cytokine construct of claim 15, wherein the one or more substitution(s) further comprise the amino acid mutation C125A compared to SEQ ID NO:1.

17. The targeted cytokine construct of claim 9, wherein the IL-2 polypeptide comprises the amino acid sequence of SEQ ID NO:1 with one of the following sets of amino acid substitutions (relative to the sequence of SEQ ID NO: 1): R38E and F42A; R38D and F42A; F42A and E62Q; R38A and F42K; R38E, F42A, and N88S; R38E, F42A, and N88A; R38E, F42A, and N88G; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

18. The targeted cytokine construct of claim 9, wherein the IL-2 polypeptide comprises an amino acid sequence that is at least about 85% identical to a sequence selected from the group consisting of SEQ ID Nos:11-90.

19. The targeted cytokine construct of claim 9, wherein said IL-2 polypeptide comprises a sequence that is at least about 75% identical to a sequence selected from the group consisting of SEQ ID Nos. 43, 48, 52, 49, and 156.

20. The targeted cytokine construct of claim 8, wherein the cytokine is the IL-7 polypeptide, or a functional fragment thereof, or a variant thereof.

21. The targeted cytokine construct of claim 20, wherein the IL-7 polypeptide exhibits reduced binding affinity by at least about 50% to an IL-7Ra polypeptide comprising the amino acid sequence of SEQ ID NO: 94, compared to binding affinity of a wild-type IL-7 polypeptide comprising the amino acid sequence of SEQ ID NO: 91 to the IL-7Ra polypeptide.

22. The targeted cytokine construct of claim 21, wherein the IL-7 polypeptide exhibits reduced binding affinity by at least about 50% to an IL-2Rg polypeptide comprising the amino acid sequence of SEQ ID NO: 4, compared to binding affinity of a wild-type IL-7 polypeptide comprising the amino acid sequence of SEQ ID NO: 91 to the IL-2Rg polypeptide.

23. The targeted cytokine construct of any one of claims 20-22, wherein the IL-7 polypeptide comprises the sequence of SEQ ID NO: 91, with one or more substitution relative to SEQ ID NO: 91.

24. The targeted cytokine construct claim 23, wherein the one or more substitutions are at positions selected from the positions: K10, Q11, S14, V15, V18, Q22, L35, N36, D74, L77, L80, K81, E84, 188, R133, Q136, E137, T140, and N143, and K144.

25. The targeted cytokine construct of claim 24, wherein the substitution in position K81 is K81A and the substitution in position T140 is K140A.

26. The targeted cytokine construct of claim 8, wherein the cytokine is the IL-10 polypeptide, or a functional fragment thereof, or a variant thereof.

27. The targeted cytokine construct of claim 26, wherein the IL-10 polypeptide exhibits reduced binding affinity by at least about 50% to an IL-10RA polypeptide comprising the amino acid sequence of SEQ ID NO: 96, compared to binding affinity of a wild-type IL-10 polypeptide comprising the amino acid sequence of SEQ ID NO: 95 to the IL-10RA polypeptide.

28. The targeted cytokine construct of claim 27, wherein the IL-10 polypeptide exhibits increased binding affinity by at least about 50% to an IL-10RB polypeptide comprising the amino acid sequence of SEQ ID NO: 97, compared to binding affinity of a wild-type IL-10 polypeptide comprising the amino acid sequence of SEQ ID NO: 95 to the IL-10RB polypeptide.

29. The targeted cytokine construct of claim 26, wherein the IL-10 polypeptide comprises the sequence of SEQ ID NO: 95, with one or more substitution relative to SEQ ID NO: 95.

30. The targeted cytokine construct of claim 29, wherein the IL-10 polypeptide comprises an amino acid sequence selected from the group consisting of SEQ ID 99-112.

31. The targeted cytokine construct of claim 8, wherein the cytokine is the IL-21 polypeptide, or a functional fragment thereof, or a variant thereof.

32. The targeted cytokine construct of claim 31, wherein the TL-21 polypeptide exhibits reduced binding affinity by at least about 50% to an IL-21R polypeptide comprising the amino acid sequence of SEQ ID NO: 93, compared to binding affinity of a wild-type IL-21 polypeptide comprising the amino acid sequence of SEQ ID NO: 92 to the IL-21R polypeptide.

33. The targeted cytokine construct of claim 32, wherein the IL-21 polypeptide exhibits reduced binding affinity by at least about 50% to an TL-2Rg polypeptide comprising the amino acid sequence of SEQ ID NO: 4, compared to binding affinity of a wild-type IL-21 polypeptide comprising the amino acid sequence of SEQ ID NO: 92 to the TL-2Rg polypeptide.

34. The targeted cytokine construct of claim 31, wherein the IL-21 polypeptide comprises the sequence of SEQ ID NO: 115, with one or more substitution relative to SEQ ID NO: 115.

35. The targeted cytokine construct of claim 34, wherein the substitution in one or more positions are selected from the positions: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115.

36. The targeted cytokine construct of any one of claims 1-35, wherein the engineered cell comprises at least one of: a T cell expressing a T cell receptor (a TCR-T cell), a gamma delta T cell, a pluripotent stem cell derived T cell, or an induced pluripotent stem cell derived T cell, a natural killer cell (NK cell), a pluripotent stem cell derived NK cell, or an induced pluripotent stem cell (iPSC) derived NK cell, a T cell engineered to express a chimeric antigen receptor (a CAR-T cell), a CD8-positive T cell, a CD4-positive T cell, a cytotoxic T cell,
a tumor infiltrating lymphocyte, a CAR-NK cell, a gamma delta T cell, a myeloid cell, a hematopoietic lineage cell, a hematopoietic stem and progenitor cell (HSC), a hematopoietic multipotent progenitor cell (MPP), a pre-T cell progenitor cell, a T cell progenitor cell, a NK cell progenitor cell.

37. The targeted cytokine construct of any one of claims 1-36, wherein the targeted cytokine construct is suitable for administering to a subject in combination with a therapy comprising the engineered cell.

38. The targeted cytokine construct of claim 37, wherein the engineered cell is autologous to the subject.

39. The targeted cytokine construct of claim 38, wherein the engineered cell is allogenic to the subject.

40. The targeted cytokine construct of any one of claims 37-39, wherein the subject is human.

41. The targeted cytokine construct of claim 40, wherein the subject has a cancer.

42. The targeted cytokine construct of claim 41, wherein the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

43. A pharmaceutical composition: comprising a targeted cytokine construct according to any one of claims 1-42, and at least one of: a pharmaceutically acceptable excipient, carrier, or diluent, or any combination thereof.

44. The pharmaceutical composition of claim 43, further comprising a population of engineered cells.

45. A cell therapy kit comprising a pharmaceutical composition that comprises a targeted cytokine construct according to any one of claims 1-42, and instructions specified for administering the targeted cytokine construct to a subject.

46. The cell therapy kit of claim 45, further comprising a pharmaceutical composition that comprises the population of engineered cells and instructions specified for administering the population of engineered cells to the subject.

47. The cell therapy kit of claim 46, wherein the pharmaceutical composition that comprises the targeted cytokine construct and the pharmaceutical composition that comprises the population of engineered cells are for sequential or simultaneous administration.

48. A method of treating a condition in a subject, the method comprising administering to the subject a therapeutic regimen comprising: (a) an engineered cell and (b) a targeted cytokine construct that comprises:
(i) a cell binding domain that binds a receptor or domain exogenously introduced into the engineered cell; and
(ii) a cytokine protein or a functional fragment or a variant thereof.

49. The method of claim 48, wherein the targeted cytokine construct selectively activates a population of engineered cells with 10-fold or greater potency as compared to activation of a population of non-engineered cells.

50. The method of claim 49, wherein administering the targeted cytokine construct results in an increase in activation of a population of engineered cells, as compared to the activation of a population of non-engineered cells.

51. The method of claim 50, wherein the activation is measured by a pSTAT5 or a pSTAT3 activation assay.

52. The method of claim 48, wherein administering the targeted cytokine construct results in an increase in expansion and/or proliferation of a population of engineered cells, as compared to the expansion and/or proliferation of a population of non-engineered cells.

53. The method of claim 48, wherein administering the targeted cytokine construct results in an increased in vivo persistence of a population of engineered cells, as compared to the in vivo persistence of a population of non-engineered cells.

54. The method of any one of claims 50-53, wherein the non-engineered cells do not express: the CAR, the TCR, or the tag molecule.

55. The method of claim 48, wherein administering the targeted cytokine construct results in an increase in activation, expansion and/or proliferation of a population of engineered cells, as compared to the activation, expansion and/or proliferation of the population of engineered cells, when administered without the targeted cytokine construct.

56. The method of claim 55, wherein the expansion and/or proliferation is in vivo or in vitro.

57. The method of claim 48, wherein administering the targeted cytokine construct results in an increased in vivo persistence of a population of the engineered cells, as compared to the in vivo persistence of the population of engineered cells, when administered without the targeted cytokine construct.

58. The method of claim 57, wherein the in vivo persistence of the population of engineered cells comprises a period of about 15 days, about 30 days to about a year.

59. The method of claim 48, wherein administering the targeted cytokine construct reduces a rate and/or extent of exhaustion of a population of the engineered cells, as compared to the rate and/or extent of exhaustion of the population of engineered cells, when administered without the targeted cytokine construct.

60. The method of claim 48, wherein administering the targeted cytokine construct results in selective potentiation of the engineered cells, allowing enhanced specific enrichment of a population of the engineered cells, as compared to specific enrichment of a population of engineered cells when administered with an untargeted cytokine or a functional fragment or a variant thereof.

61. The method of claim 48, wherein administering the targeted cytokine construct does not increase count of Treg cells in a biological sample isolated from the subject, as compared to the count of Treg cells in a biological sample isolated from a subject who has been administered an untargeted cytokine or a functional fragment or a variant thereof.

62. The method of claim 61, wherein the biological sample is at least one of: a tumor biopsy or peripheral blood.

63. The method of any one of claims 48-62, wherein the subject is previously administered a pre-conditioning regimen.

64. The method of claim 63, wherein administering the targeted cytokine construct allows for reduction in at least one of: severity or duration of the pre-conditioning regimen.

65. The method of claim 64, wherein pre-conditioning regimen is used to decrease the endogenous lymphocyte population so as to allow a population of the engineered cell to expand.

66. The method of any one of claims 63-65, wherein the pre-conditioning regimen comprises administering a lymphodepletion agent.

67. The method of claim 66, wherein administering the targeted cytokine construct reduces the extent of lymphodepletion required for engraftment of the engineered cell.

68. The method of any one of claims 63-67, wherein the pre-conditioning regimen involves administering a chemotherapeutic agent to the subject.

69. The method of claim 68, wherein the chemotherapeutic agent is at least one of: fludarabine and cyclophosphamide.

70. The method of any one of claims 63-69, wherein the pre-conditioning regimen comprises a radiation treatment.

71. The method of any one of claims 63-70, wherein the pre-conditioning regimen comprises administering a depleting antibody.

72. The method of claim 71, wherein the depleting antibody is alemtuzumab.

73. The method of any one of claims 48-62, wherein the subject is not administered a pre-conditioning regimen.

74. A method of eliminating the need for administering, or minimizing the severity of, a pre-conditioning regimen prior to administering an engineered cell therapy, the method comprising, administering to a subject a therapeutic regimen comprising:
(a) an engineered cell; and
(b) a targeted cytokine construct that comprises: (i) a cell binding domain that binds a receptor or domain exogenously introduced into the engineered cell; and (ii) a cytokine protein or a functional fragment or a variant thereof,

75. The method of claim 74, wherein the subject has not been administered a pre-conditioning regimen.

76. The method of claim 75, wherein the targeted cytokine construct selectively activates engineered cells with 10-fold or greater potency as compared to activation of non-engineered cells.

77. The method of claim 76, wherein the activation is measured by a pSTAT5 or pSTAT3 activation assay.

78. The method of claim 76 or 77, wherein the non-engineered cells do not comprise a receptor or domain exogenously introduced into the cells.

79. The method of any one of claims 48-78, wherein the targeted cytokine construct is administered within or within about 2 days, 3 days, 6 days, 12 days, 15 days, 30 days, 60 days or 90 days or more following administering the engineered cell.

80. The method of any one of claims 48-79, wherein the targeted cytokine construct is administered simultaneously with administering the engineered cell.

81. The method of any one of claims 48-80, wherein an effective dose of the engineered cell in the therapeutic regimen is lower than that of a reference therapeutic regimen that comprises administering the engineered cell but does not comprise administering the targeted cytokine construct.

82. The method of claim 81, wherein the effective dose of the engineered cell in the therapeutic regimen is at least about 1.5× to about 1000× lower than the effective dosage of the engineered cell in the reference therapeutic regimen.

83. A method of increasing the efficacy of an engineered cell therapy in a subject, the method comprising: administering to a subject a therapeutic regimen comprising:
(a) the engineered cell; and
(b) a targeted cytokine construct comprising: (i) a cell binding domain that binds a receptor or domain exogenously introduced into the engineered cell; and (ii) a cytokine protein or a functional fragment or a variant thereof, thereby increasing the efficacy of the engineered cell therapy in the subject.

84. The method of claim 83, wherein the targeted cytokine construct selectively activates engineered cells with 10-fold or greater potency as compared to activation of non-engineered cells.

85. The method of claim 83 or 84, wherein the non-engineered cells do not comprise a receptor or domain exogenously introduced into the cells.

86. The method of any one of claims 76-85, wherein the wherein the potency is measured by a pSTAT5 or pSTAT3 activation assay.

87. A method of treating a subject who has undergone a loss of B cell aplasia, the method comprising: administering to the subject a targeted cytokine construct that comprises:
(i) a cell binding domain that binds a receptor or domain exogenously introduced into the engineered cell; and
(ii) a cytokine protein or a functional fragment or a variant thereof.

88. A method of treating a condition or disease, comprising administering to the subject a therapeutic regimen comprising: (a) an engineered cell; and (b) a targeted cytokine construct that comprises:
(i) a cell binding domain that binds a receptor or domain exogenously introduced into the engineered cell; and
(ii) a cytokine protein or a functional fragment or a variant thereof, wherein the administering the targeted cytokine construct allows for reducing an effective dose of the engineered cell in the therapeutic regimen, relative to the effective dose of the engineered cell in a reference therapeutic regimen that comprises administering the engineered cell but does not comprise the targeted cytokine construct.

89. The method of claim 88, wherein the effective dose of the engineered cell in the therapeutic regimen is at least about 1.5× to about 1000× lower than the effective dosage of the engineered cell in the reference therapeutic regimen.

90. The method of any one of claims 48-89, wherein the engineered cell is provided in a composition, and wherein the composition is generated at a point-of-care and is administered into a patient without culturing the population of cells.

91. A method of targeting an engineered cell in a subject, the method comprising, administering to the subject a targeted cytokine construct comprising a cell binding domain and a modified cytokine or a functional fragment or a variant thereof, wherein the engineered cell expresses (i) a receptor for the modified cytokine or a functional fragment or a variant thereof, and (ii) a target antigen for the cell binding domain.

92. A method of enriching a population of an engineered cell in a subject, the method comprising: administering to the subject a targeted cytokine construct comprising a cell binding domain and a modified cytokine or a functional fragment or a variant thereof, wherein the engineered cell expresses (i) a receptor for the modified cytokine or a functional fragment or a variant thereof, and (ii) a target antigen for the cell binding domain.

93. The method of claim 91 or 92, wherein the engineered cell is generated in vivo in the subject.

94. The method of claim 93, wherein the subject has previously been administered a nucleic acid carrier, comprising a nucleic acid that expresses a chimeric antigen receptor (CAR) or a T cell receptor protein (TCR).

95. The method of claim 94, wherein the nucleic acid carrier is at least one of: a linear polynucleotide, a polynucleotide associated with ionic or amphiphilic compounds, a plasmid, and a virus.

96. The method of claim 94, wherein the nucleic acid carrier is a nanocarrier.

97. The method of claim 94, wherein the nucleic acid carrier is a viral vector, wherein the viral vector is at least one of: a Sendai viral vector, an adenoviral vector, an adeno-associated virus vectors, a retroviral vector, or a lentiviral vector.

98. The method of any one of claims 94-97, wherein the nucleic acid is a DNA or an RNA.

99. The method of claim 98, wherein the RNA is a messenger RNA (mRNA).

100. The method of any one of claims 94-99, wherein the nucleic acid carrier further comprises a targeting moiety for targeting an immune cell.

101. The method of claim 100, wherein the immune cell comprises, a myeloid cell, a T cell or an NK cell.

102. The method of claim 101, wherein the T cell comprises a T lymphocyte.

103. The method of claim 101 or 102, wherein the T cell or the NK cell is induced by the vector or the nucleic acid carrier, to generate the engineered cell in vivo in the subject.

104. The method of any one of claims 92-103, wherein the administering the targeted cytokine construct results in an increase in activation, expansion and/or proliferation of a population of engineered cells generated in vivo, as compared to the activation, expansion and/or proliferation of the population of the engineered cells generated in vivo, when the subject is not administered the targeted cytokine construct.

105. The method of any one of claims 92-104, wherein the administering the targeted cytokine construct results in an increase in persistence of a population of engineered cells generated in vivo, as compared to the persistence of the population of the engineered cells generated in vivo, when the subject is not administered the targeted cytokine construct.

106. The method of any one of claims 92-105, wherein administering the targeted cytokine construct reduces a rate and/or extent of exhaustion of a population of engineered cells generated in vivo, as compared to the rate and/or extent of exhaustion of the population of the engineered cell generated in vivo, when administered without the targeted cytokine construct.

107. The method of any one of claims 92-106, wherein administering the targeted cytokine construct results in selective potentiation of the engineered cells generated in vivo, allowing enhanced specific enrichment of a population of the engineered cells generated in vivo, as compared to specific enrichment of a population of engineered cells when administered with an untargeted cytokine or a functional fragment or a variant thereof.

108. The method of any one of claims 92-107, wherein administering the targeted cytokine construct does not increase count of Treg cells in a biological sample isolated from the subject, as compared to the count of Treg cells in a biological sample isolated from a subject who has been administered an untargeted cytokine or a functional fragment or a variant thereof.

109. The method of claim 108, wherein the biological sample is at least one of: a tumor biopsy or peripheral blood.

110. The method of any one of claims 105-109, wherein the persistence of the population of engineered cells comprises a period of at least about 30 days to about a year.

111. A method of enriching a population of an engineered cell, the method comprising: contacting the population of the engineered cell with a targeted cytokine construct comprising a cell binding domain and a modified cytokine or a functional fragment or a variant thereof, wherein the engineered cell expresses (i) a receptor for the modified cytokine or a functional fragment or a variant thereof, and (ii) a target antigen for the cell binding domain.

112. The method of any one of claims 48-111, wherein the cytokine is selected from the group consisting of: IL-2, IL-7, IL-10, IL-15, and IL-21, or a functional fragment thereof, or a variant thereof, or any combinations thereof.

113. The method of any one of claims 48-112, wherein said cytokine is at least one of: (i) an IL-2Rβγ agonist polypeptide that binds to and/or activates an IL-2Rβ polypeptide comprising the amino acid sequence of SEQ ID NO: 3; and (ii) an IL-2Rβγ polypeptide agonist polypeptide that binds to and/or activates an IL-2Rγ polypeptide comprising the amino acid sequence of SEQ ID NO: 4.

114. The method of claim 112 or 113, wherein the cytokine is an IL-2 polypeptide, or a functional fragment thereof, or a variant thereof.

115. The method of claim 114, wherein said the IL-2 polypeptide exhibits reduced binding affinity by at least about 50% to an IL-2Rα polypeptide comprising the amino acid sequence of SEQ ID NO: 2, compared to binding affinity of a wild-type IL-2 polypeptide comprising the amino acid sequence of SEQ ID NO:1 to the IL-2Rα polypeptide.

116. The method of claim 115, wherein the IL-2 polypeptide exhibits reduced binding affinity by at least about 50% to an IL-2Rβ polypeptide comprising the amino acid sequence of SEQ ID NO: 3, and/or reduced binding affinity by at least about 50% to an IL-2Rγ polypeptide comprising the amino acid sequence of SEQ ID NO:4, compared to binding affinity of a wild-type IL-2 polypeptide comprising the amino acid sequence of SEQ ID NO: 1 to the IL-2Rβ polypeptide.

117. The method of claim 112, wherein the cytokine is an IL-7 polypeptide that exhibits reduced binding affinity by at least about 50% to an IL-7Ra polypeptide comprising the amino acid sequence of SEQ ID NO: 94, compared to binding affinity of a wild-type IL-7 polypeptide comprising the amino acid sequence of SEQ ID NO: 91 to the IL-7Ra polypeptide.

118. The method of claim 117, wherein the IL-7 polypeptide exhibits reduced binding affinity by 50% or more to an IL-2Rg polypeptide comprising the amino acid sequence of SEQ ID NO: 4, compared to binding affinity of a wild-type IL-7 polypeptide comprising the amino acid sequence of SEQ ID NO: 91 to the IL-2Rg polypeptide.

119. The method of claim 112, wherein the cytokine is a IL-10 polypeptide that exhibits reduced binding affinity by at least about 50% to an IL-10RA polypeptide comprising the amino acid sequence of SEQ ID NO: 96, compared to binding affinity of a wild-type IL-10 polypeptide comprising the amino acid sequence of SEQ ID NO: 95 to the IL-10RA polypeptide.

120. The method of claim 119, wherein the IL-10 polypeptide exhibits increased binding affinity by at least about 50% to an IL-10RB polypeptide comprising the amino acid sequence of SEQ ID NO: 97, compared to binding affinity of a wild-type IL-10 polypeptide comprising the amino acid sequence of SEQ ID NO: 95 to the IL-10RB polypeptide.

121. The method of claim 120, wherein the cytokine is a IL-21 polypeptide that exhibits reduced binding affinity by 50% or more to an IL-21R polypeptide comprising the amino acid sequence of SEQ ID NO: 93, compared to binding affinity of a wild-type IL-21 polypeptide comprising the amino acid sequence of SEQ ID NO: 92 to the IL-21R polypeptide.

122. The method of claim 121, wherein the IL-21 polypeptide exhibits reduced binding affinity by at least about 50% to an IL-2Rg polypeptide comprising the amino acid sequence of SEQ ID NO:4, compared to binding affinity of a wild-type IL-21 polypeptide comprising the amino acid sequence of SEQ ID NO: 92 to the IL-2Rg polypeptide.

123. The method of claim 112, wherein the cytokine is an IL-2 polypeptide that comprises the sequence of SEQ ID NO:1 with one or more amino acid substitutions relative to SEQ ID NO:1, and wherein the one or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130.

124. The method of claim 123, wherein the one or more substitution(s) comprise an F42A or F42K amino acid substitution relative to SEQ ID NO:1.

125. The method of claim 124, wherein the one or more substitution(s) further comprise an R38A, R38D, R38E, E62Q, E68A, E68Q, E68K, or E68R amino acid substitution relative to SEQ ID NO:1.

126. The method of claim 125, wherein the one or more substitution(s) further comprise an H16E, H16D, D20N, M23A, M23R, M23K, S87K, S87A, D84L, D84N, D84V, D84H, D84Y, D84R, D84K, N88A, N88G, N88S, N88T, N88R, N88I, N88D, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1.

127. The method of claim 126, wherein the one or more substitution(s) further comprise the amino acid mutation C125A compared to SEQ ID NO:1.

128. The method of claim 112, wherein the cytokine is an IL-2 polypeptide that comprises an amino acid sequence that is at least about 85% identical to a sequence selected from the group consisting of SEQ ID Nos:11-90.

129. The method of claim 112, wherein the cytokine is an IL-2 polypeptide that comprises an amino acid sequence that is at least about 85% identical to a sequence selected from the group consisting of SEQ ID NOs: 43, 48, 52, 49, and 156.

130. The method of claim 112, wherein the cytokine is an IL-2 polypeptide that comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and N88G; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

131. The method of claim 112, wherein the cytokine is an IL-7 polypeptide, or a functional fragment or a variant thereof.

132. The method of claim 131, wherein the IL-7 polypeptide comprises the sequence of SEQ ID NO: 91, with one or more substitution relative to SEQ ID NO: 91.

133. The method of claim 132, wherein the substitution in one or more positions are selected from the positions: K10, Q11, S14, V15, V18, Q22, L35, N36, D74, L77, L80, K81, E84, 188, R133, Q136, E137, T140, and N143, and K144.

134. The method of claim 133, wherein the substitution in position K81 is K81A and the substitution in position T140 is K140A.

135. The method of claim 112, wherein the cytokine is an IL-10 polypeptide, or a functional fragment or a variant thereof.

136. The method of claim 135, wherein the IL-10 polypeptide comprises the sequence of SEQ ID NO: 95, with one or more substitution relative to SEQ ID NO: 95.

137. The method of claim 136, wherein the mutant IL-10 polypeptide comprises an amino acid sequence selected from the group consisting of SEQ ID 99-112.

138. The method of any one claim 112, wherein the cytokine is an IL-21 polypeptide, or a functional fragment thereof, or a variant thereof.

139. The method of claim 138, wherein the IL-21 polypeptide comprises the sequence of SEQ ID NO: 115, with one or more substitution relative to SEQ ID NO: 115.

140. The method of claim 139, wherein the substitution in one or more positions are selected from the positions: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115.

141. The method of any one of claims 48-140, wherein the engineered cell comprises at least one of: a T cell expressing a T cell receptor (a TCR-T cell), a gamma delta T cell, a pluripotent stem cell derived T cell, or an induced pluripotent stem cell derived T cell, a natural killer cell (NK cell), a pluripotent stem cell derived NK cell, or an induced pluripotent stem cell (iPSC) derived NK cell, a T cell engineered to express a chimeric antigen receptor (a CAR-T cell), a CD8-positive T cell, a CD4-positive T cell, a cytotoxic T cell, a tumor infiltrating lymphocyte, a CAR-NK cell, a gamma delta T cell, a myeloid cell, a hematopoietic lineage cell, a hematopoietic stem and progenitor cell (HSC), a hematopoietic multipotent progenitor cell (MPP), a pre-T cell progenitor cell, a T cell progenitor cell, a NK cell progenitor cell.

142. The method of claim 141, wherein the engineered cell is autologous to the subject.

143. The method of claim 142, wherein the engineered cell is allogenic to the subject.

144. The method of any one of claims 48-143, wherein the subject is human.

145. The method of any one of claims 48-144, wherein the subject has a cancer.

146. The method of claim 145, wherein the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

147. A targeted cytokine construct for use in a combination therapy with an engineered cell, the fusion protein comprising (i) a cell binding domain, and (ii) a cytokine protein or a functional fragment or a variant thereof, wherein the cell binding domain:
(a) comprises an antibody or an antigen binding fragment thereof that is specific for a receptor or domain exogenously expressed on the surface of the engineered cell;
(b) comprises an antibody or an antigen binding fragment thereof that is specific for a domain of an antigen binding protein expressed on the engineered cell;
(c) is specific for a tag, wherein the tag is co-expressed by the engineered cell or is part of a receptor expressed by the engineered cell;
(d) is a domain from an antigen targeted by the engineered cell; or
(e) comprises any combinations of (a)-(d).

148. The targeted cytokine construct of claim 147, wherein the receptor expressed by the engineered cell is a chimeric antigen receptor (CAR) or a T cell receptor (TCR).

149. The targeted cytokine construct of claim 148 wherein the cytokine is selected from the group consisting of: IL-2, IL-7, IL-10, IL-15, and IL-21, or a functional fragment thereof, or a variant thereof, or any combinations thereof.

150. The targeted cytokine construct of claim 149, wherein the cytokine is an IL-2 polypeptide, or a functional fragment thereof, or a variant thereof.

151. The targeted cytokine construct of claim 150, wherein the IL-2 polypeptide comprises the sequence of SEQ ID NO:1 with one or more amino acid substitutions relative to SEQ ID NO:1, and wherein the one or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, I92, T123, Q126, S127, I129, and S130.

152. The targeted cytokine construct of claim 151, wherein the one or more substitution(s) comprise an F42A or F42K amino acid substitution relative to SEQ ID NO:1.

153. The targeted cytokine construct of claim 151 or 152, wherein the one or more substitution(s) further comprise an R38A, R38D, R38E, E62Q, E68A, E68Q, E68K, or E68R amino acid substitution relative to SEQ ID NO:1.

154. The targeted cytokine construct of any one of claims 151-153, wherein the one or more substitution(s) further comprise an H16E, H16D, D20N, M23A, M23R, M23K, S87K, S87A, D84L, D84N, D84V, D84H, D84Y, D84R, D84K, N88A, N88G, N88S, N88T, N88R, N88I, N88D, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1.

155. The targeted cytokine construct of any one of claims 151-154, wherein the one or more substitution(s) further comprise the amino acid mutation C125A compared to SEQ ID NO:1.

156. The targeted cytokine construct of any one of claims 151-155, wherein the IL-2 polypeptide comprises an amino acid sequence that is at least about 85% identical to a sequence selected from the group consisting of SEQ ID Nos:11-90.

157. The targeted cytokine construct of claim 150, wherein the IL-2 polypeptide comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and N88G; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

158. The targeted cytokine construct of claim 149, wherein the cytokine is an IL-7 polypeptide that comprises the sequence of SEQ ID NO: 91, with one or more substitution relative to SEQ ID NO: 91.

159. The targeted cytokine construct of claim 158, wherein the substitution in one or more positions are selected from the positions: K10, Q11, S14, V15, V18, Q22, L35, N36, D74, L77, L80, K81, E84, 188, R133, Q136, E137, T140, and N143, and K144.

160. The targeted cytokine construct of claim 159, wherein the substitution in positions K81 and T140 are K81A and T140A.

161. The targeted cytokine construct of claim 149, wherein the cytokine is an IL-10 polypeptide comprises the sequence of SEQ ID NO: 95, with one or more substitution relative to SEQ ID NO: 95.

162. The targeted cytokine construct of claim 161, wherein the IL-10 polypeptide comprises an amino acid sequence selected from the group consisting of SEQ ID 99-112.

163. The targeted cytokine construct of claim 149, wherein the cytokine is an IL-21 polypeptide, or a functional fragment thereof, or a variant thereof.

164. The targeted cytokine construct of claim 163, wherein the IL-21 polypeptide comprises the sequence of SEQ ID NO: 115, with one or more substitution relative to SEQ ID NO: 115.

165. The targeted cytokine construct of claim 164, wherein the IL-21 polypeptide comprises the sequence of SEQ ID NO: 115, or a sequence comprising an amino acid substitution at one or more positions selected from the group consisting of positions: R5, 18, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 115.

166. The targeted cytokine construct of any one of claims 147-165, wherein the tag co-expressed by the engineered cell is an EGFRt tag.

167. The targeted cytokine construct of any one of claims 147-166, wherein the antigen targeted by the engineered cell is selected from the group consisting of: a neoepitope from a tumor-associated antigen, TSHR, CD19, CD123, CD22, CD30, CD171, CS-1, CLL-1, CD33, EGFRvlll, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11 Ra, PSCA, PRSS21, VEGFR2, LewisY, CD24, PDGFR-beta, SSEA-4, CD20, Folate receptor alpha, ERBB2 (Her2/neu), MUC1 EGFR, NCAM, Prostase, PAP, ELF2M, Ephrin B2, IGF-I receptor, CAIX, LMP2, gp100, ber-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51 E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1 B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1.

168. The targeted cytokine construct of any one of claims 147-167, wherein the engineered cell comprises at least one of: a T cell expressing an alpha beta T cell receptor, a gamma delta T cell, an NK T cell, a regulatory T cell, a pluripotent stem cell derived T cell, or an induced pluripotent stem cell derived T cell, a natural killer cell (NK cell), a pluripotent stem cell derived NK cell, or an induced pluripotent stem cell (iPSC) derived NK cell, a T cell engineered to express a chimeric antigen receptor (a CAR-T cell), a T cell engineered to express a T cell receptor (a TCR-T cell), a CD8-positive T cell, a CD4-positive T cell, a cytotoxic T cell, a tumor infiltrating lymphocyte, an NK cell engineered to express a chimeric antigen receptor (a CAR-NK cell), an NK T cell engineered to express a chimeric antigen receptor (a CAR-NK T cell), a myeloid cell, a hematopoietic lineage cell, a hematopoietic stem and progenitor cell (HSC), a hematopoietic multipotent progenitor cell (MPP), a pre-T cell progenitor cell, a T cell progenitor cell, a NK cell progenitor cell.

169. A method of treating a cancer, the method comprising administering a targeted cytokine construct according to any one of claims 147-168, in a combination therapy with the engineered cell.

170. The method of claim 169, further comprising administering an additional therapeutic agent

171. The method of claim 169 or 170, wherein the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantlecell lymphoma (MCL), Transformed follicular lymphoma (TFL), Primary mediastinal B cell lymphoma (PMBCL), Multiple myeloma, Hairy cell lymphoma/leukemia, lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

172. A pharmaceutical composition: comprising a targeted cytokine construct according to any one of claims 147-168, and at least one of: a pharmaceutically acceptable excipient, carrier, or diluent, or any combination thereof.

173. The pharmaceutical composition of claim 172, further comprising a population of the engineered cell.

174. A cell therapy kit that has a pharmaceutical composition that comprises a targeted cytokine construct according to any one of claims 147-168 and instructions specified for administering the targeted cytokine construct to a subject.

175. The cell therapy kit of claim 174, further comprising a pharmaceutical composition that comprises a population of the engineered cells and instructions specified for administering the population of engineered cells to the subject.

176. The cell therapy kit of claim 175, wherein the pharmaceutical composition that comprises the targeted cytokine construct and the pharmaceutical composition that comprises the population of engineered cells are for sequential or simultaneous administration.
